SEQUENCE LISTING (1 sequence)

SEQ ID NO: 1 - DNA, 20 bp, Artificial Sequence
  Artificial Oligonucleotide
  CpG (1)..(20)
tccatgacgttcctgatgct

(19) United States
(12) Patent Application Publication
(10) Pub. No.: US 2020/0392289 A1
(43) Pub. Date: Dec. 17, 2020

(54) Biodegradable Polymer and Use Thereof

(71) Applicant: ANP Technologies, Inc., Newark, DE (US)

(72) Inventors: Ray Yin, Wilmington, DE (US); Jing Pan, Newark, DE (US); Zhiying Zou, Newark, DE (US); Xiaoyu Wang, Allendale, NJ (US); Kai Qi, Wilmington, DE (US)

(21) Appl. No.: 16/762,938

(22) PCT Filed: Nov. 12, 2018
(86) PCT No.: PCT/US2018/060575

§ 371 Date: May 10, 2020

Related U.S. Application Data
(60) Provisional application No. 62/584,128, filed on Nov. 10, 2017.

(51) Int. Cl. C08G 73/02; A61K 39/00; A61K 31/7068; A61K 31/015; A61K 31/282; A61K 33/243; A61K 9/51

(52) U.S. Cl. CPC C08G 73/0206; A61K 39/001114; A61K 31/7068; A61K 9/08; A61K 31/282; A61K 33/243; A61K 9/5146; A61K 31/015

(57) ABSTRACT

This invention is directed to a biodegradable polymer that can be degraded in vivo. The biodegradable polymer comprises a biodegradable polymer segment having at least a biodegradable bond and two or more cationic components, wherein each of said cationic components is covalently attached to the biodegradable polymer segment and the two cationic components/molecules are separated by at least one biodegradable bond in the backbone. The biodegradable polymer can be used for targeting desired ceils in vivo including T cells, NK (natural killer) ceils, cancer cells, or a combination thereof, delivering genes, DNA, oligodeoxynucleotide, oligonucleotide, RNA, mRNA, RNAi, siRNA, microRNA, protein, peptide, antibody, fragment of an antibody, small molecule drug including chemotherapy drugs, or other bioactive agents into cells, or being used as a vaccine or drug for treating a disease such as a cancer in a subject.

### FIELD OF THE INVENTION

This invention is directed to a non-crosslinked biodegradable cationic polymer. This invention is further directed to a composition comprising the biodegradable cationic polymer for use in gene and drug delivery, treatment of diseases, as well as detection and diagnostics.

### BACKGROUND

Polymers have been tested as a non-viral carrier for delivering nucleic acids including DNA and RNA, proteins, or other large or small molecules into cells for therapeutic purposes or for modifying cells. Cationic polymers are suitable for delivering genes and other materials into cells due to their positive charge under physiological conditions for the ease of complexation with nucleic acids and for targeting cells that are typically negatively charged.

Polymers can include polyethylene glycol (PEG), polyethylenimine (PEI), polyalkylamine, polyallylamine, polylysine (PLK), polypeptide, chitosan, polysaccharide or polysaccharide functionalized with amino or imino functions, poly(dimethylaminoethyl methacrylate), or co-polymers. Poly(β-amino ester)s that are biodegradable can also be useful due to their ability to bind DNA, promote cellular uptake, facilitate escape from the endosome, and allow for DNA release in the cytoplasm (Green et al., Acc. Chem. Res. 41:749-759, 2008). Poly(β-amino ester)s with diamine end-modification can also be used for effective gene delivery (Zugates et al., Mol. Ther. 15:1306-1312, 2007). Some of acrylate-terminated polymers or amine monomer-terminated polymers may also be useful. Polyethylenimine (PEI) is one of the polymers that shows potential and utility for gene delivery partly due to the cationic structure that can bind to DNA for delivery of a DNA into cells (see Boussif et al., Proc. Natl. Acad. Sci. USA 92:7297-301, 1995; U.S. Pat. No. 6,013,240, granted on Jan. 11, 2000). Polyethylenimine (PEI) can be linear (LPEI) or branched (BPEI).

Although some polymers, such as PEIs, have shown promise over other polymers, higher cytotoxicity and lower efficacy when compared to viral methods are still challenges facing the industry. In addition, since PEI polymers are not biodegradable, significant toxicities have been observed in various in vivo studies (Moghimi et al., Mol. Ther. 11:990, 2005).

Therefore, continued needs exist for better polymers that can deliver genes and drugs at high effectiveness and with low in vitro and in vivo toxicity.

### SUMMARY

The present invention is directed to a biodegradable polymer comprising two or more cationic components and at least one biodegradable bond formed by biomolecules, wherein the cationic components are separated by at least one of the biodegradable bond and the cationic components are attached to the biomolecules covalently.

The present invention is also directed to a bioactive composition comprising a biodegradable polymer of this invention and at least one bioactive agent. The biodegradable polymer and the bioactive agent are linked with covalent bonds or non-covalent linkages. The bioactive agent is an RNA, an mRNA, an RNAi, a siRNA, an microRNA, an oligonucleotide, a DNA, an oligodeoxynucleotide, a protein, a peptide, an antibody, a fragment of an antibody, a chemical compound, a chemotherapy drug, a small molecule drug, or a combination thereof. The bioactive composition can be a pharmaceutical composition for treating a disease of a subject in need thereof.

In embodiments, a bioactive composition of interest is employed in a combination with one or more other bioactive agents, which may or may not be associated with a biodegradable polymer of interest. Any one or more other bioactive agents can be used, such as, a nucleic acid, a small molecule drug, a biological or large molecule drug, a protein and so on. The one or more other bioactive agents can be an existing drug or pharmaceutical composition.

The present invention is further directed to a method for delivering a bioactive agent into a biosystem using a biodegradable polymer of this invention. The biosystem can be selected from cells in vitro, cells in vivo, nuclei of cells, cytoplasm of cells, extracellular matrix, tissues, body fluid of a subject, blood of the subject, one or more organs of the subject, one or more tumors of the subject, or a combination thereof.

The present invention is also directed to a method for treating a disease of a subject in need thereof. The method can comprise the steps of: associating a biodegradable polymer of this invention and at least one bioactive agent to produce a bioactive composition; and introducing the bioactive composition to the subject. The biodegradable polymer and at least one bioactive agent can be associated via one or more covalent bonds or non-covalent linkages.

The present invention is further directed to a system for an assay, wherein the system comprises a biodegradable polymer of this invention and a bioactive agent, wherein the assay is an immunoassay, an enzymatic assay, a nucleic acid based assay, a hybridization assay, or combination thereof. The said immunoassay can be a sandwich, competitive, direct, indirect, sequential immunoassay, or a combination thereof. The enzymatic assay can be an enzyme inhibition assay. The nucleic acid assay can be a PCR, gene sequencing, hybridization assay of nucleic acids, hybridization assay of proteins and nucleic acids, or a combination thereof. The assay can also be a hybridization of proteins or peptides, hybridization of peptides, or hybridization of proteins or peptides with oligos or nucleic acids.

### BRIEF DESCRIPTION OF THE FIGURES

FIGS. 1A-I. Schematic examples of polymer segments. FIG. 1A: An example of a linear polymer segment. FIG. 1B: An example of a branched polymer segment.

FIG. 1C: Two or more polymer segments each having at least one cationic component attached to a biodegradable polymer. FIG. 1D: a polymer segment comprising polyglutamic acid (PLE). FIG. 1E: A polymer segment comprising polycarbohydrate including polysaccharide. FIG. 1F-FIG. 1H: Examples of biodegradable polymers having a polymer core and one or more polymer segments attached to the polymer core. FIG. 1I: An example of a biodegradable polymer having a polymer core and polylysine (PLK) segments. “D” is a polymer core. P₁ through Pᵢ each is a cationic component, m and m′ each is an integer and m+m′≥2.

FIGS. 2A-0. Schematic examples of biodegradable polymers. A polymer backbone is attached to: FIG. 2A, Symmetrically branched polymer cationic components; FIG. 2B, Asymmetrically randomly branched polymer cationic components; FIG. 2C, Asymmetrically regularly branched polymer cationic components; FIG. 2D: Branched polymer cationic components; FIG. 2E, Linear polymer cationic components; FIG. 2F, Branched and/or linear polymer cationic components; FIG. 2G, Comb polymer cationic components; FIG. 2H, Dendronized polymer cationic components and FIG. 2I, Star-branched polymer cationic components. FIG. 2J: A polymer core with biodegradable polymer segments attached thereon and further modified with branched cationic components. FIG. 2K: A polymer core with biodegradable polymer segments attached thereon and further modified with one or more ethylamine groups. FIG. 2L: A polymer core with cationic biodegradable polymer segments further modified with branched cationic polymer segments. FIG. 2M: A polymer core with branched biodegradable polylysine segments. FIG. 2N: A polymer core with linear biodegradable polylysine segments further modified with polyethyleneimine (PEI). FIG. 2O: Covalently linked polymers each having a polymer core and biodegradable polymer segments. A biodegradable polymer can be either linear or branched. Only some polymer components are shown. Drawings may not be to scale. As used herein and in other figures, each small open circle represents one or more biodegradable bonds; a solid or a shaded circle represents a polymer core; and lines represent polymer chains each having multiple carbon or other elements. For simplicity, “EI” or “PEI” as shown in some of the figures can represent a single residue of an ethyleneimine (EI), a segment of polymerized ethyleneimine (PEI) having two or more polyethyleneimine, a reacted ethylenediamine, a reacted propylenediamine (PI), a polypropyleneimine (PPI) or a combination thereof.

FIGS. 3A-D. Schematic structural examples of biodegradable polymers. FIG. 3A: Branched poly-L-lysine attached with EI (or PEI) attached to lysine residues. FIG. 3B: Linear poly-L-lysine with PEI (or EI) attached to lysine residues. FIG. 3C: An example of a linear poly-L-lysine backbone with cationic components attached thereon. FIG. 3D: An example of two cationic components attached to the same point in the polymer backbone. Not all combinations or components are shown.

FIGS. 4A-F. Examples of reaction schemes. FIG. 4A: Poly-L-lysine PEI polymers (PLK-PEI) using bromoethylamine. FIG. 4B-4C: PLK-EI and PLK-PEI that have a bridging molecule (also referred to as a linking molecule or a linker) between the polylysine and the cationic component. FIG. 4D: Poly-L-glutamic acid PEI polymer (PLE-PEI). FIG. 4E: Polysaccharide PEI polymer (Polysaccharide-PEI). FIG. 4F: PEI-PLK polymer having one ethyleneimine layer with protected ethylamine (PEI-PLK-EI) or with multiple layers of ethyleneimine or random polyethyleneimine (PEI-PLK-P(EI)).

FIGS. 5A-N. Schematic illustrations of examples of biodegradable polymers with different cationic components. FIG. 5A and FIG. 5B: Polyethyleneimine (PEI). FIG. 5C and FIG. 5D: Polyamidoamine (PAMAM). FIG. 5E: Polyethyleneimine (PEI) modified with polyamidoamine (PAMAM). FIG. 5F: Polyethyleneimine (PEI) modified with polylysine. FIG. 5G and FIG. 5H: Polyethyleneimine (PEI) modified with lysine (PLK) having lysine-lysine peptide bonds and additional one or more layers of polyethyleneimine (PEI), GO (PLK1): with one layer of lysine; G1 (PLK2): with 2 layers of lysine; G2 (PLK3): with 3 layers of lysine. FIG. 5I and FIG. 5J: Examples of dendrimers produced from a polypropyleneimine (PPI) dendrimer modified with polylysine and further modified with one layer of ethylamine, Den(PPI-PLK-EI), or two or more layers of ethylamine, Den(PPI-PLK-PEI) (only some end amine groups and only one dendrimer branch are shown for simplicity). FIG. 5K and FIG. 5L: Examples of dendrimers produced from polyamidoamine (PAMAM) dendrimer modified with polylysine and further modified with one layer of ethylamine, Den(PAMAM-PLK-EI) or two or more layers of ethylamine, Den(PAMAM-PLK-PEI). FIG. 5M and FIG. 5N: Schematic outlines of examples of dendrimers having a polymer core modified with biomolecule polymers having at least one biodegradable bond and further modified with cationic components such as ethyleneimine (EI) or Polyethyleneimine (PEI), or polyamidoamine (PAMAM). For simplicity, not all bonds, groups, chemical reagents, reaction steps, reaction conditions or components are shown. MA: methylacrylate. EDA: ethylene diamine.

FIG. 6. An example of a biodegradable polymer conjugated with a drug molecule camptothecin. CDI is carbonyl diimidazole. EDA is ethylene diamine. A drug containing a hydroxyl group can be reacted with CDI to generate an imidazole carbamate modified drug. This drug can then be linked with an amine group of a biodegradable polymer through an amidation reaction in a solvent or buffer, such as DMSO.

FIGS. 7A-C. Examples of biodegradable polymers conjugated with drug molecules or additional functional groups. FIG. 7A: A polymer reacted with succinyl anhydride (SA) that can be used to further attach additional biomolecules, such as a drug. FIG. 7B: Polymer conjugated with a drug molecule via an imidazole group of a CDI as described in the legend of FIG. 6. FIG. 7C: A polymer reacted with an epoxide, such as a glycidol, that can be used to further attach additional one or more biomolecules, such as drug molecules. G: glycidol. SA: succinyl anhydride.

FIGS. 8A-I. Schematic structural illustrations of examples of biodegradable polymers linked with bioactive agents via covalent bonds. One or more bioactive agents are linked to: FIG. 8A, symmetrically branched polymeric cationic components attached to a biodegradable polymer backbone; FIG. 8B, asymmetrically randomly branched polymeric cationic components attached to a biodegradable polymer backbone; FIG. 8C, asymmetrically regularly branched polymeric cationic components attached to a biodegradable polymer backbone; FIG. 8D, branched polymeric cationic component; FIG. 8E, linear cationic components attached to a biodegradable polymer backbone; FIG. 8F, branched and/or linear branched cationic components on a biodegradable polymer backbone; FIG. 8G, comb polymeric cationic components on a biodegradable polymer backbone; FIG. 8H, dendronized polymeric cationic components on a biodegradable polymer backbone, and FIG. 8I, star-branched polymeric cationic components on a biodegradable polymer backbone. A biodegradable polymer backbone can be either linear or branched. Only some polymer components are shown. Drawings may not be to scale. Each solid square represents one or more bioactive agents.

FIGS. 9A-E. Schematic illustrations of examples of biodegradable polymers having bioactive agents with covalent bonds. FIG. 9A: Multiple molecules of a single type of bioactive agent (with an optional IgG as a targeting agent or a second bioactive agent). FIG. 9B: Multiple molecules of multiple types of bioactive agents. FIG. 9C: Antibody, IgG, antibody fragment, or antigen fragment. FIG. 9D: Covalently linked bioactive agent (BA) is depicted attached to a biodegradable polymer of interest using a linker. A linker can be attached, for example, to a phosphate group or a nitrogen group of a bioactive agent and the other end of the linker is joined to a biodegradable polymer. FIG. 9E: A linker is attached to two biodegradable polymers, and the linker also is bound to bioactive agent. Open squares, solid squares, triangles each represents a different bioactive agent.

FIGS. 10A-I. Schematic examples of nanoparticles having bioactive agents. FIG. 10A: Bioactive agent molecules are mostly inside the polymer/nanoparticles. FIG. 10B: Bioactive agent molecules are mostly at the surface of polymer/nanoparticle. FIG. 10C: Bioactive agent molecules are distributed throughout the polymer/nanoparticle. FIG. 10D: Covalently linked bioactive agent (BA) at the surface of the biodegradable polymer. FIG. 10E-FIG. 10F: Covalently linked RNA or DNA at the surface of the biodegradable polymer. FIG. 10G-FIG. 10I: BA, RNA including mRNA, and DNA are non-covalently encapsulated with biodegradable polymer nanoparticles.

FIGS. 11A-F. Schematic illustrations of examples of biodegradable polymers/nanoparticles comprising non-covalently linked bioactive agents. FIG. 11A: Small molecule bioactive agent (solid squares). FIG. 11B: Antibody or purified IgG or fragment thereof. FIG. 11C: Large molecule bioactive agent such as a biological or large molecule drug. FIG. 11D: The same biodegradable polymer as in C, however, the polymer may exhibit, for example, different charge under different environments, such as at different pH conditions. The charge can vary before, during or after formation of the nanoparticles. FIG. 11E: Bioactive agents and Biodegradable polymer having a polymer core and biodegradable polymer segments. FIG. 11F: Bioactive agent and covalently linked polymer cores and biodegradable polymer segments.

FIGS. 12A-G. Functional assays. FIG. 12A: Toxicity assays of branched dendritic polyethyleneimine (bPEI 25K, MW 25 KDa) and modified dendrimer polyethyleneimine Den(PEI18-PLK2-EI/PEI) having a MW 1.8 KDa PEI core (PEI 1.8K) modified with 2 layers of polylysine and one or more layers of ethylamine (EA). FIG. 12B: FACS profile of H460 cells only (Cell Control). FIG. 12C: FACS profile of cells treated with 20-mer oligo linked Cy3 dye alone without polymer (Cell+D Control). FIG. 12D: FACS profile of cells treated with 20-mer oligo linked Cy3 dye and a dendrimer Den(PEI12-PLK3-EI168) (Cell+D P1). FIG. 12E: FACS profile of cells treated with the oligo linked Cy3 dye and a Den(PEI12-PLK2-EI-C18) dendrimer (Cell+D P2). FIG. 12F: Florescent intensity profile of live cells alone (Cell), transfected with the oligo linked Cy3 control (Cell+D) and oligo linked Cy3 plus the dendrimer Den(PEI12-PLK3-EI168) (Cell+D P1). FIG. 12G: Florescent intensity profile of live cells alone (Cell), transfected with the oligo linked Cy3 control (Cell+D) and oligo linked Cy3 plus the dendrimer Den(PEI12-PLK2-EI-C18) (Cell+D P2). The enclosed areas represent live cells as indicated.

### DETAILED DESCRIPTION

Features and advantages of the present invention will be more readily understood, by those of ordinary skill in the art, from reading the following detailed description. It is to be appreciated that certain features of the invention, which are described above and below in the context of separate embodiments, may also be provided in combination in a single embodiment. Conversely, various features of the invention that are, for brevity, described in the context of a single embodiment, may also be provided separately or in any combination or sub-combination. In addition, references in the singular may also include the plural (for example, “a” and “an” may refer to one, or one or more) unless the context specifically states otherwise.

Use of numerical values in the various ranges specified in this application, unless expressly indicated otherwise, are stated as approximations as though minimum and maximum values within the stated ranges were both proceeded by the word, “about.” In this manner, slight variations above and below the stated ranges can be used to achieve substantially the same results as values within the ranges. Also, disclosure of ranges is intended as a continuous range including every value between the minimum and maximum values and including the minimum and maximum cited values.

The term “buffer” refers to a compound or a combination of compounds and other agent or agents, a solution of the compound or a combination of the compounds and agent or agents which maintain solution pH in a certain range. It can include succinate (sodium or potassium), histidine, phosphate (sodium or potassium), citrate (sodium or potassium), Tris (tris(hydroxymethyl) aminomethane), diethanolamine, carbonate, bicarbonate, citrate (sodium), glucose, and the like. A buffer can be have a pH in a desired range, for example, a buffer can be adjusted to have a pH that is within the range of from about 4.5 to about 8.5. A buffer system that is compatible with lyophilization can be preferred. A buffer system that is suitable for pharmaceutical use is further preferred.

The term “biomolecule”, “biomolecules” or other grammatic variations used herein refers to molecules appearing in or found in biological systems, such as plants, animals, bacteria, virus, etc. Biomolecules can comprise amino acid, polymerized amino acid, polypeptide comprising one or more different types of amino acids, such as polymerized lysine, polymerized aspartic acid, polymerized glutamic acid, polycarbohydrate such as polysaccharides, or a combination thereof.

The term “sugar”, “sugars”, “saccharide,” “polysaccharide,” “carbohydrate” or “polycarbohydrate” refers to a carbohydrate compound that can include mono-sugar, monosaccharide, disaccharide, trisaccharide, oligosaccharide, polysaccharide, or a combination thereof. Examples of sugars can include, but are not limited to, monosaccharides such as glucose, mannose, galactose, fructose and sorbose; disaccharides such as sucrose, lactose, maltose and trehalose; and trisaccharide such as raffinose. Sugar can also include other disaccharides, such as cellobiose, gentiobiose or isomaltose. Oligosaccharides and polysaccharides having a lower molecular weight and are soluble in water, such as having up to 6 monosaccharide sugar units, for example, homoglycans, may be suitable. Sugar can further include sugar alcohol, such as xylitol, glycerol, sorbitol, lactitol, isomalt, mannitol and erythritol. Polymerized monosaccharides, disaccharide, trisaccharide or other polysaccharides that comprise at least a biodegradable bond, such as a glycosidic bond can be suitable. A sugar can be the D- or the L-enantiomer/isomer. Similarly, amino acids can also be the L- or the D-enantiomer.

The term “biodegradable bond” refers to a chemical bond of a composition that can be cleaved, hydrolyzed, split, or otherwise degraded to produce a product composition that is smaller than the original composition when in contact with biomolecules such as one or more enzymes or in a biosystem, such as in cells, animal or human, body fluids, extracellular matrix, cytoplasm, cell membrane, organ, organelle, or a combination thereof. Degradation can be enzymatic or non-enzymatic. Examples of enzymes suitable for enzymatic degradation can include, but are not limited to, peptidase, aminopeptidase, esterase, lipase, glycosidase, nuclease, and the like, or a combination thereof. Examples of non-enzymatic cleavage can include, but are not limited to, acid hydrolysis such as under low pH conditions. Suitable biodegradable bond can include peptide bond, also known as an amide bond, a reducible disulfide bond, glycosidic bond, ester bond or a combination thereof.

The term “lyophilization”, “lyophilized”, or “freeze-dried” refers to a process by which material to be dried is first frozen, generally below the triple point temperature of water or a solvent, and then the ice or frozen solvent is removed by vaporization and generally by sublimation in a vacuum environment.

The term “cationic molecule”, “cationic component”, “cationic components”, “cationic moiety”, “cationic member”, “cationic polymeric component”, or “cationic unit” refers to one which under metabolic or physiological conditions is neutral or carries a positive charge, or when exposed to varying environmental conditions, carries a positive charge. Any cationic molecule compatible with physiological conditions can be used in a biodegradable polymer of interest. A cationic molecule can be an ion, a functional group, a biochemical entity, a monomer, a polymer and so on that can be attached to a biodegradable polymer and has or can have one or more cationic or positives charges. A cationic component can be a cationic polymer, a cationic polymer segment, or a cationic functional group. A cationic component can be pre-polymerized and then attached to a biodegradable polymer via a covalent bond, grow with monomers from an attachment point or a reactive site of a polymeric backbone or a polymer core of a biodegradable polymer, or a combination thereof. A cationic component can comprise functional groups, monomers or polymers, such as, one or more amine groups or a variation thereof, a compound comprising one or more amine groups, or a combination thereof. The term “amine group” or “amine” used herein refers to a primary amine, a secondary amine, a tertiary amine, or a combination thereof.

As used herein, a monomer name can be used to describe a monomer as a stand-alone monomer molecule or a residue or a reacted unit in a polymer or a polymer segment. It is understood by those skilled in the art, a monomer can have one or more functional groups or atoms reacted, removed, or changed when it becomes a residue or a reacted unit of a polymer. For example, the term “lysine” can refer to a stand-alone lysine amino acid, a lysine residue in a polypeptide, or a reacted lysine in a linear or branched polylysine polymer as understood by those skilled in the art. In another example, the term ethyleneimine (EI), ethylamine or ethylene diamine (EDA) can refer to a stand-alone monomer, a part of a monomer, a reacted residue or part of a reacted residue as a part of a polymer or a polymer component that has a —N(CH₂)₂NH—, —N(CH₂)₂NH₂ or —N(CH₂)₂N⁺H₃.

By, “physiological conditions,” or various grammatic forms thereof is meant the milieu in a biological body, such as a human, an animal, a plant, a cell, or a condition where a biological system, such as a cell or an organ can maintain its biological function. Thus, any composition that can be administered to a subject, such as a human or an animal, for purposes herein is deemed compatible with physiological conditions. A composition that is compatible with physiological conditions can be administered to a subject, such as a human or an animal, or delivered to cells or tissues, such as cultured cells or xenografted tumors.

The term “pharmaceutical formulation” or “pharmaceutical composition” refers to a preparation or a composition which is in such form for administration to a subject so as to permit active ingredients to be effective and in compliance with government regulations for use in treatment of a disease or conditions in humans or animals.

The term “pharmaceutically acceptable” in terms of an excipient, a carrier, a vehicle, an additive, or a filler is one that can be administered to a subject mammal or animal to provide an effective dose of the active ingredient and in compliance with government regulations. Ingredients listed in US Food and Drug Administration (US FDA) “Inactive Ingredients Database” or an equivalent thereof can be suitable.

The term “modified”, “modification”, “substituted”, “derived”, “derivatized” or various grammatic forms thereof is meant a change, which can be an addition, deletion or other change(s), of an entity to yield a different, non-identical entity. For example, a change in electric charge or forming a salt, removal or an addition of an atom, group, molecule and the like can be a modification. Some examples of methods for modifying a polypeptide or a polynucleotide can include exposure to iodoacetic acid, PEGylation, exposure to N-ethyl maleimide and so on. The term “modified” can also encompass a naturally occurring molecule that is altered, for example, to provide a new property. For example, a polypeptide or a polynucleotide can be constructed to contain a monomer, a residue or a base that is not normally found in polynucleotides and polypeptides, for example, a modified polynucleotide contain one or more phosphorothioate bases instead of phosphodiester bases. Some of modifications can maintain the molecule compatible with physiological conditions.

Symmetrically branched polymers (SBPs) are a class of polymers such as dendritic polymers (also referred to as dendrimers herein), including Starburst dendrimers (or Dense Star polymers) and Combburst dendrigrafts (or hyper comb-branched polymers), that have: (a) a well-defined core molecule, (b) at least two concentric dendritic layers (generations) with symmetrical (equal length) branches and branch junctures and (c) optionally, exterior surface groups, such as those including, but not limited to, amino, carboxyl, ester, aliphatic, aromatic, silicon containing, fluorine containing, sulfur containing groups, etc., derived from polyamidoamine (PAMAM)-based branched polymers and dendrimers described in U.S. Pat. Nos. 4,435,548; 4,507,466; 4,568,737; 4,587,329; 5,338,532; 5,527,524; and 5,714,166. Other examples include polyethyleneimine (PEI) dendrimers, such as those disclosed in U.S. Pat. No. 4,631,337; polypropyleneimine (PPI) dendrimers, such as those disclosed in U.S. Pat. Nos. 5,530,092; 5,610,268; and 5,698,662; Frechet-type polyether and polyester dendrimers, core shell tectodendrimers and others, as described, for example, in, “Dendritic Molecules,” edited by Newkome et al., VCH Weinheim, 1996, “Dendrimers and Other Dendritic Polymers,” edited by Frechet & Toroalia, John Wiley & Sons, Ltd., 2001; and U.S. Pat. No. 7,754,500.

Combburst dendrigrafts are constructed with a core molecule and concentric layers with symmetrical branches through a stepwise synthetic method. In contrast to dendrimers, Combburst dendrigrafts or polymers are generated with monodisperse linear polymeric building blocks (U.S. Pat. Nos. 5,773,527; 5,631,329 and 5,919,442). Moreover, the branch pattern is different from that of dendrimers. For example, Combburst dendrigrafts form branch junctures along the polymeric backbones (chain branches), while Starburst dendrimers often branch at the termini (terminal branches). Due to the living polymerization techniques used, the molecular weight distributions (M_{w}/Mₙ) of those polymers (core and branches) often are narrow. Thus, Combburst dendrigrafts produced through a graft-on-graft process are well defined with M_{w}/Mₙ ratios often approaching 1.

SBPs, such as dendrimers, are produced predominantly by repetitive protecting and deprotecting procedures through either a divergent or a convergent synthetic approach. Since dendrimers utilize small molecules as building blocks for the core and the branches, the molecular weight distribution of the dendrimers often is defined. In the case of lower generations, a single molecular weight dendrimer often is obtained. While dendrimers often utilize small molecule monomers as building blocks, dendrigrafts use linear polymers as building blocks.

Asymmetrically branched polymers (ABPs) are, particularly asymmetrically branched dendrimers or regular ABP (reg-ABP), often possess a core, controlled and well-defined asymmetrical (unequal length) branches and asymmetrical branch junctures as described in U.S. Pat. Nos. 4,289,872; 4,360,646; and 4,410,688.

A random asymmetrically branched polymer (ran-ABP) possesses: a) no core, b) functional groups both at the exterior and in the interior, c) random/variable branch lengths and patterns (i.e., termini and chain branches), and d) unevenly distributed interior void spaces.

The synthesis and mechanisms of ran-ABPs, such as those made from PEI, were reported by Jones et al., J. Org. Chem. 9, 125 (1944), Jones et al., J. Org. Chem. 30, 1994 (1965) and Dick et al., J. Macromol. Sci. Chem., A4 (6), 1301-1314, (1970)), while the synthesis of linear PEI was reported by Tomalia et. al. in Macromolecules 24: 1435-1438. Ran-ABP, such as those made of POX, i.e., poly(2-methyloxazoline) and poly(2-ethyloxazoline), as reported by Litt (J. Macromol. Sci. Chem. A9(5), 703-727 (1975)) and Warakomski (J. Polym. Sci. Polym. Chem. 28, 3551 (1990)). The synthesis of ran-ABPs often can involve a one-pot divergent or a one-pot convergent method.

Homopolymer refers to a polymer or a polymer backbone composed of the same repeat unit, that is, the homopolymer is generated from the same monomer (e.g., PEI linear polymers, POX linear polymers, PEI dendrimers, polyamidoamine (PAMAM) dendrimers or POX dendrigrafts and randomly branched polymers). The monomer can be a simple compound, a complex or an assemblage of compounds, such as macromonomers or oligomers, where the assemblage or complex can be the repeat unit in the homopolymer. In some circumstances, those molecules could be classified as copolymers. One or more of the monomer or complex monomer components can be modified, substituted, derivatized and so on, for example, modified to carry a functional group. Such molecules are homopolymers for the purposes of the instant disclosure as the backbone is composed of a single simple (or monosaccharide) or complex monomer.

Dendronized polymers are linear polymer backbones having repeat dendron (a partial dendrimer or a dendritic wedge) units attached thereon.

Linear polymer backbones comprising biodegradable bonds, such as polypeptides, such as poly-L-lysine, poly-L-glutamic acid, poly-L-aspartic acid, or a combination thereof, polysaccharides and so on can be suitable. Each of the dendron units can be branched, tree-like fragments. For example, a branched polyethyleneimine (PEI) or polypropyleneimine (PPI) can be suitable as a dendron unit and can be suitable as a cationic component of a biodegradable polymer disclosed herein.

This invention is directed to a biodegradable polymer comprising two or more cationic components and at least one biodegradable bond formed by biomolecules, wherein the cationic components are separated by at least one of biodegradable bond and the cationic components are attached to the biomolecules covalently.

At least one of the cationic components can be covalently attached to a biomolecule directly free from a bridging molecule or a bridging atom (also herein referred to as a linking molecule or a linker). In one example, an alkyleneimine, a polymerized alkyleneimine, an ethyleneimine, a polymerized ethyleneimine (PEI), a propyleneimine, a polymerized propyleneimine (PPI), a polymerized amidoamine (PAMAM), a tris(2-aminoethyl)amine (TREN), a polymerized tris(2-aminoethyl)amine, a polyalkylamine, a polyallylamine, or a combination thereof, is attached to a polylysine (PLK) at one or more amine groups of lysine molecules directly. In another example, a polyethyleneimine (PEI) is attached to a polylysine (PLK) at one amine end group of a lysine residue directly. In another example, an ethylamine (EA) is attached to a polylysine (PLK) at one amine end group a lysine residue directly. In yet another example, a propylamine is attached to a polylysine (PLK) at one amine end group a lysine residue directly. In a further example, one or more additional lysine residues can be attached to a polylysine (PLK) at one amine end group directly.

A cationic component can also be covalently attached to a biomolecule via a bridging molecule having 1-20 bridging atoms. The bridging molecule can have 1-20 bridging atoms in one example, 1-18 bridging atoms in another example, 1-16 bridging atoms in another example, 1-15 bridging atoms in another example, 1-14 bridging atoms in another example, 1-13 bridging atoms in another example, 1-12 bridging atoms in another example, 1-11 bridging atoms in another example, 1-10 bridging atoms in another example, 1-9 atoms in yet another example, 1-8 atoms in yet another example, 1-7 atoms in another example, 1-6 atoms in yet another example, 1-5 atoms in yet another example, 1-4 atoms in yet another example, 1-3 atoms in yet another example and 1-2 atoms in a further example. A bridging molecule can comprise one or more carbon, nitrogen, phosphorus, sulfur, oxygen atoms or a combination thereof. In a further example, a bridging molecule can comprise a reacted SMCC, a maleimide, a disulfide linkage, a sulfur linkage, a phosphate, a phosphoric acid, a carboxylic acid, an alkyl, an aryl, an alkene, an aromatic carbon, a cyclic carbon, or a combination thereof. In an even further example, a cationic component can be covalently attached to a biomolecule via a bridging molecule consisting of a reacted molecule selected from the group consisting of a SMCC, a maleimide (MAL), a Traut's reagent, a disulfide linkage (—S—S—), a sulfur linkage (—S—), a phosphate, a phosphoric acid, a C1-C20 alkyl, a C1-C5 carboxylic acid, a C1-C20 alkene and a combination thereof. In yet another example, a cationic component can be covalently attached to a biomolecule via a heterologous bridging molecule having at least two different elements selected from carbon, nitrogen, sulfur, phosphorous and oxygen. In an even further example, a cationic component can be covalently attached to a biomolecule via a heterologous bridging molecule that is a reaction product of SMCC and Traut's reagent. In a yet further example, a cationic component can be covalently attached to a biomolecule via a heterologous bridging molecule that is a reaction product of maleimide (MAL) and Traut's reagent. In a further example, a cationic component can be covalently attached to a biomolecule via a heterologous bridging molecule that is a reaction product of maleimide (MAL) and a cysteamine. In another example, a cationic component can be covalently attached to a biomolecule via a heterologous bridging molecule that is a reaction product of SMCC and a cysteamine. Additional chemistries linking the cationic component to biomolecules are described in Bioconjugate Techniques (3rd Edition) by Greg Hermanson, Elsevier, 2013.

The biodegradable polymer of this invention is non-crosslinked. The biodegradable polymer disclosed herein is soluble in aqueous solutions. The biodegradable polymer disclosed herein is soluble in physical conditions. The biodegradable polymer is free from gelled or crosslinked form under physical conditions.

The polymeric backbone or polymer core, either linear or branched, can be produced by practicing known materials and methods or can be purchased commercially. Similarly, a cationic molecule can be purchased or constructed as known in the art, and attached to the backbone or core using known chemistries, methods and materials.

A biodegradable polymer of this invention can comprise polylysine (PLK) and two or more cationic components each is covalently linked to one of lysine residues via a bridging molecule that is a reaction product of a SMCC and a thio group, a SMCC and a Traut's reagent, a maleimide and a thio group, or a maleimide and a Traut's reagent.

The term “reacted” used herein and throughout this application refers to a molecule that is reacted to become a part of a polymer with appropriate changes in molecular structure, for example, with one or more atoms or functional groups reacted, changed, added or removed from the original molecule or group. A reacted molecule can also be referred to a residue of the molecule in a polymer.

In a biodegradable polymer of this invention, at least one of the cationic components comprises at least one end amine group selected from —N(CH₂)NH₂, —N(CH₂)₂NH₂, —N(CH₂)₃NH₂, —N(CH₂)N⁺H₃, —N(CH₂)₂N⁺H₃, —N(CH₂)₃N⁺H₃, or a combination thereof. The biodegradable polymer of this invention can also comprise one or more cationic components each consists of end amine group selected from the group consists of —N(CH₂)₂NH₂, —N(CH₂)₃NH₂, —N(CH₂)₂N⁺H₃, —N(CH₂)₃N⁺H₃, and a combination thereof. The biodegradable polymer can comprise —N(CH₂)₂NH₂ group as an end amine group of the cationic components in one example, —N(CH₂)₃NH₂ group as an end amine group of the cationic components in another example, —N(CH₂)₂N⁺H₃ group as an end amine group of the cationic components in yet another example, —N(CH₂)₃N⁺H₃, group as an end amine group of the cationic components in yet another example, a combination of —N(CH₂)₂NH₂, —N(CH₂)₃NH₂, —N(CH₂)₂N⁺H₃ and —N(CH₂)₃N⁺H₃ as end amine groups in a further example. The cationic components can comprise at least one end amine group —NCH₂NH₂ or —NCH₂N⁺H₃, that can be provided, for example from a reactive methylenediamine or methylenediamine dihydrochloride.

A biodegradable bond can be in the backbone of a biodegradable polymer, at least one side chain of a biodegradable polymer, or both the backbone and at least one side chain of a biodegradable polymer. The biodegradable bond can comprise a peptide bond, a glycosidic bond, or a combination thereof, and wherein the biomolecules can comprise amino acid or sugar. A biodegradable bond can also comprise an ester bond, a reducible disulfide bond, or a combination thereof. In brief, a biodegradable bond can be a peptide bond, also known as an amide bond; a reducible disulfide bond, such as a disulfide bond that can be reduced under reducing conditions or by an enzyme, including disulfide bond that can be reduced in a biosystem such as a cell, extracellular matrix, cell culture, in blood, in plasma, or other in vivo conditions or a combination thereof; a glycosidic bond; a ester bond that can be degraded by an enzyme or hydrolysis; or a combination thereof. The glycosidic bonds can be α-1,3 bonds, α-1,4 or α-1,6 bonds, or a combination thereof. The peptide bond and the glycosidic bond, or a combination thereof, can be preferred. The biodegradable bond can be degraded by enzymes, such as a peptidase, a glycoside hydrolase, such as a cellulase, a hemicellulase, an amylase, a viral neuraminidase, a glycosidase, a mannosidase, esterase, hydrolases, or a combination thereof. The biodegradable bond can also be degraded by non-enzymatic cleavages, such as, but not limited to, acid hydrolysis such as under low pH conditions that is common in lysosomes.

In one example, a biodegradable polymer comprises a linear polylysine having at least one lysine-lysine biodegradable peptide bond in its polymer backbone. In another example, a biodegradable polymer is a branched polylysine having at least one lysine-lysine biodegradable peptide bond in its polymer backbone and at least one peptide bond in its side chains. In yet another example, a biodegradable polymer comprises one or more polylysine side chains each having one or more lysine-lysine peptide bonds. In a further example, a biodegradable polymer is a linear polysaccharide having at least one sugar-sugar biodegradable glycosidic bond in its polymer backbone. In another example, a biodegradable polymer is a branched polysaccharide having at least one sugar-sugar biodegradable glycosidic bond in its polymer backbone and at least one glycosidic bond in its side chains. In yet another example, a biodegradable polymer comprises one or more polysaccharide side chains each having one or more glycosidic bonds.

The biomolecules can comprise a lysine (Lys), a modified lysine, a glutamic acid (Glu), a modified glutamic acid, an aspartic acid (Asp), a modified aspartic acid, arginine (Arg), a modified arginine, or a combination thereof. Examples of modified lysine can include, but are not limited to, Lys (lysine) treated by succinylation, malonylation or acylation, practicing known methods. The modified lysine can be purified from in vivo post-translation modification or from chemical modification. Examples of modified glutamic acid (Glu) can include Glu treated by hydroxylation, alkylation, or a combination thereof. Commercially available poly-L-lysine and poly-L-glutamic acid, such as those available from Sigma-Aldrich, can be suitable.

The biodegradable polymer of this invention can comprise a linear polylysine, a branched polylysine, a linear polyglutamic acid, a branched polyglutamic acid, alinear polyarginine, a branched polyarginine, alinear or branched polymerized sugar, disaccharide, polysaccharide, or a combination thereof. The branched polylysine include dendritic polylysine. The biodegradable polymer can comprise one type of amino acid or a combination of a plurality of different amino acids, such as a poly(lysine, glutamic acid) or poly(lysine, arginine). In one example, biomolecules can consist of lysine and polylysine.

The biodegradable polymer can have a molecular weight (MW) in a range of from 1,000 to about 1,500,000. As used herein, molecular weight is of Dalton (Da) as a default or kilodalton (KDa, K or KD) when specified. The biodegradable polymer of this invention can have a MW in a range of from 1,000 to 1,500,000 in one example, 2,000 to 1,500,000 in another example, 4,000 to 1,500,000 in yet another example, 8,000 to 1,500,000 in yet another example, 10,000 to 1,500,000 in yet another example, 15,000 to 1,500,000 in yet another example, 25,000 to 1,500,000 in yet another example, 1,000 to 100,000 in yet another example, 1,000 to 50,000 in yet another example, 1,000 to 40,000 in yet another example, 1,000 to 30,000 in yet another example, 1,000 to 25,000 in yet another example, 1,000 to 20,000 in yet another example, 1,000 to 15,000 in yet another example, 1,000 to 10,000 in yet another example and 1,000 to 8,000 in a further example. In an even further example, a biodegradable polymer of this invention can have a MW in a range of from 2,000 to 60,000.

In any of biodegradable polymers of this invention, each of the cationic components can be independently a linear polymer, a branched polymer, a hyperbranched polymer, a graft polymer, a block polymer, a dendrimer, or a combination thereof. Each of the cationic components can have molecular weight (MW) in a range of from about 40 Da to about 5,000 Da, 40 to 4,000 in another example, 40 to 3,000 in yet another example, 40 to 2,600 in yet another example, 40 to 2,400 in yet another example, 40 to 2,000 in yet another example, 40 to 1,600 in yet another example, 40 to 1,200 in yet another example, 40 to 800 in yet another example, 40 to 600 in yet another example, 40 to 300 in yet another example and 40 to 180 in a further example.

Any of biodegradable polymers of this invention disclosed herein can comprise alinear or a branched biodegradable polymer segment comprising in a range of from 2 to 1,000 polymerized lysine residues, polymerized glutamic acid residues, polymerized aspartic acid residues, or a combination thereof. A biodegradable polymer segment of the biodegradable polymer of this invention can have 2 to 1,000 polymerized lysine residues in one example, 2 to 500 polymerized lysine residues in another example, 2 to 100 polymerized lysine residues in yet another example, 2 to 50 polymerized lysine residues in yet another example, 2 to 10 polymerized lysine residues in yet another example, 2 to 5 polymerized lysine residues in yet another example, 2 to 3 polymerized lysine residues in yet another example and 2 polymerized lysine residues in a further example.

In any biodegradable polymers of this invention, at least one of the cationic components can comprise lysine, polylysine, alkyleneimine, polymerized alkyleneimine, ethyleneimine, polymerized ethyleneimine (PEI), propyleneimine, polymerized propyleneimine (PPI), polymerized amidoamine (PAMAM), tris(2-aminoethyl)amine (TREN), polymerized tris(2-aminoethyl)amine, polyalkylamine, polyallylamine or a combination thereof. In some examples, at least one of the cationic components can consist of lysine, polylysine, alkyleneimine, polymerized alkyleneimine, ethyleneimine, polymerized ethyleneimine (PEI), propyleneimine, polymerized propyleneimine (PPI), polymerized amidoamine (PAMAM), tris(2-aminoethyl)amine (TREN), polymerized tris(2-aminoethyl)amine, polyalkylamine, polyallylamine and a combination thereof. In yet further examples, at least one of the cationic components can comprise ethyleneimine, ethylamine, polyethyleneimine, propyleneimine, propylamine, polypropyleneimine, or a combination thereof. In yet a further example, at least one of the cationic components can comprise at least one end amine group provided by ethyleneimine, ethylamine, propyleneimine, propylamine, or a combination thereof. For simplicity, any of the molecules listed above are meant to be in a form reacted or otherwise incorporated onto the biodegradable polymer covalently as understood by those skilled.

The cationic components can be produced by reacting a modified amine, such as a bromoalkylamine (or alkylamine bromide, other suitable haloalkylamines), for example, a bromoethylamine, a bromopropylamine, or a combination thereof with reactive amines of the biomolecules. Each cationic component can comprise primary amine (1°), secondary amine (2°), tertiary amine (3°) groups, or a combination thereof. For example, bromoethylamine can react with reactive amine groups, such as primary and secondary amine groups, of lysine to produce a cationic component comprising one or more ethyleneimine groups directly attached to one lysine residue. The reaction can be controlled with molar ratios of reactants, such as bromoethylamine:reactive amine ratio in a range of from about 0.1:1 to about 10:1, preferably in a range of from 0.5:1 to 5:1, further preferred in a range of from 1:1 to 2:1. A protected ethyleneimine bromide can also be suitable to add only one ethylamine group onto one lysine residue of the biodegradable polymer. With a protected ethyleneimine bromide, multiple ethyleneimine groups can be added to multiple lysine residues of the polymer. The product then can be deprotected to generate an amine group. The newly added amine groups can further react with bromoethylamine to add additional amine groups producing a biodegradable polymer comprising at least one lysine residue having two or ethyleneimine groups (PEI) on one of its amine groups, i.e., two or more layers of ethyleneimine groups.

A biodegradable polymer of this invention can further comprise at least one reducible disulfide bond. For example, a cystamine dihydrochloride can be used in some of the reactions disclosed herein to produce a reducible disulfide bond in the backbone or a side chain of a polymer by practicing known methods. The reducible disulfide bond can be in a polymer backbone, a polymer side chain, or a combination thereof.

A biodegradable polymer of this invention can comprise one or more polymer segments each having a formula

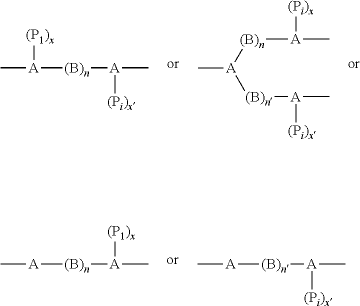

or a combination thereof, with a proviso that the biodegradable polymer comprises at least two of the cationic components,

wherein,

n and n′ each is an integer ≥0;

x and x′ each is an integer ≥1;

A is one of said biomolecules;

B is selected from A, a linear polymer component comprising A, a branched polymer component comprising A, a dendrimer component comprising A, or a combination thereof, wherein each of said polymer segments comprises at least one of the biodegradable bond; and

P₁ through Pᵢ are the cationic components, and wherein the P₁ through Pᵢ are the same or different.

In examples, n and n′ each can be in a range of from 0 to 10,000, x and x′ each can be in arrange of from 1 to 20. x and x′ each can be selected based on the number of reactive sites available on the biomolecule A for attaching one or more cationic components. In one example, when A is a lysine residue of a polylysine, it can have one or two —NH₂ groups (a terminal lysine residue can have 2 —NH₂ groups, while a non-terminal lysine residue can have 0 or one —NH₂ group) and each can attach one or two cationic components thereon, therefore, x and x′ each can be 1 to 4. The unit “A” can also be referred to as “group A”, “A unit”, “A component” or “A moiety”, and an A-A bond is biodegradable.

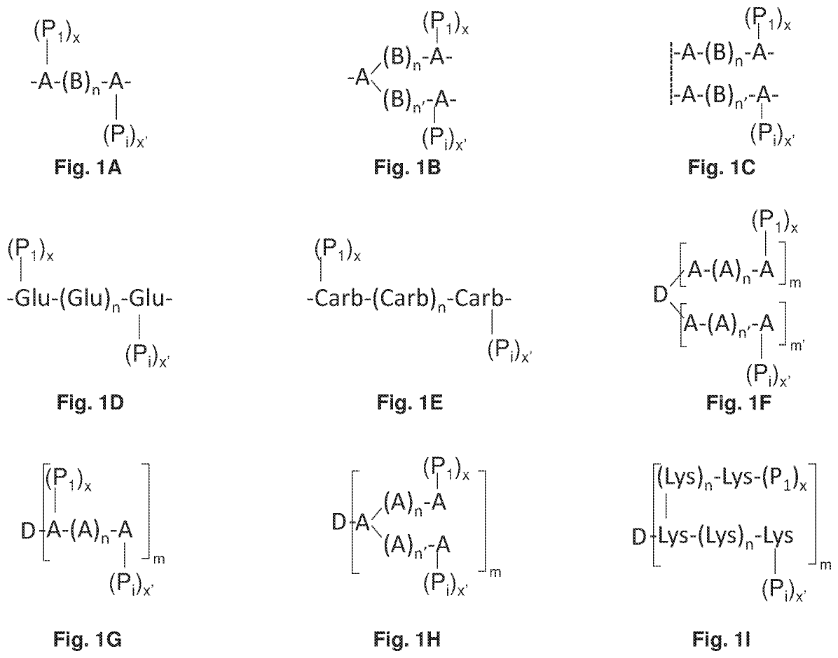

A biodegradable polymer can comprise one or more linear polymer segments (FIG. 1A). In one example, a biodegradable polymer can comprise one or more polymer segments having the formula:

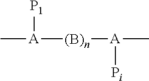

wherein,

P₁ through Pᵢ can be cationic components comprising a polymerized ethylamine, polymerized propylamine, or a combination thereof. The polymer can have more than one cationic component on biomolecules. “A” and “B” can both be a same biomolecule, such as an amino acid, for example, lysine, glutamic acid, aspartic acid, or a combination of different biomolecules.

Any of biomolecules of this invention can comprise at least a branched polymer segment, such as schematically shown in FIG. 1B. A biodegradable polymer can comprise two or more same or different polymer segments each having one or more cationic components. These polymer segments can be covalently inked in the polymer with polymeric bonds, with a proviso that at least two of the cationic components are separated by at least one biodegradable bond, such as schematically shown in FIG. 1C. Once a biodegradable bond is cleaved or otherwise degraded in a biosystem, the cationic components can be released from the polymer leading to, for example, reduced toxicity to the biosystem, since, in examples, each of the cationic components are designed to be of a molecular weight that has low or no toxicity to the biosystem.

In one embodiment, the group B is the same as the group A. The biodegradable polymer can comprise polymer segments having the formula:

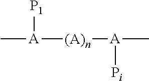

with the A-A bond being biodegradable. The biodegradable polymer can comprise polymer segments having polymerized amino acids, such as polymerized L-lysine, polymerized D-lysine, polymerized L-glutamic acid (polyGlu, or PLE), polymerized L-aspartic acid (PLD), polymerized D-aspartic acid, polymerized D-glutamic acid, and polycarbohydrate such as polysaccharides (FIG. 1D-FIG. 1E), or a combination thereof. The L-amino acids can be preferred. Polymer segments comprising linear or branched poly-L-lysine (herein referred to as polylysine or PLK), linear or branched poly-L-aspartic acid (also referred to as polyaspartic acid or PLD), linear or branched poly-L-glutamic acid (also referred to as poly(glutamic acid) or PLE), or a combination thereof, can be used. Polymer segments comprising polysaccharide can also be suitable. A co-polymer polymerized from various combinations of amino acids, such as, a combination of lysine, glutamic acid or aspartic acid can also be suitable. For example, in the formula above, “A” can be a lysine residue and “B” can be a glutamic acid residue or poly-L-glutamic acid. Conversely, “A” can be a glutamic acid residue (PLE) and “B” can be a lysine residue or poly-L-lysine. The poly-L-lysine and the poly-L-glutamic acid can be linear or branched. For simplicity, when describing polymers herein, unless specifically defined, the term lysine, lysine residue, glutamic acid, aspartic acid, glutamic acid residue, polylysine, poly-L-lysine, polyglutamic acid, poly-aspartic acid, poly-L-glutamic acid, the group “A” or “B”, or the like, refers to a residue, a reactant, a reacted form of a respective compound, a residue or a group in a polymer. As used herein, a poly(amino acid), such as a polylysine, poly(glutamic acid) or poly(aspartic acid) refers to a polymerized segment comprising 2 or more amino acid residues, either as a linear or a branched polymer or polymer segment.

The polymer segments can be repeating units or non-repeating units for forming a biodegradable polymer. The cationic components can be separated by a certain number or a varying number of biodegradable bonds, in alinear or a branched polymer backbone, side chain, or both backbone and side chain.

In one example, both A and B are lysine. A biodegradable polymer comprising one or more polymer segments with the formula:

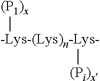

wherein Lys-(Lys)ₙ-Lys can comprise a linear or branched polylysine. Any of cationic components disclosed herein can be suitable for P₁ through Pᵢ. One lysine residue can have one or two reactive —NH₂ groups and thus can react to up to 4 cationic components, i.e., x and x′ can each be from 1 to 4. In one example, alinear polylysine having 8 lysine residues can have up to 9 reactive —NH₂ groups, and therefore, can react to up to 18 cationic components P₁ through P₁₈, such as ethylamine or a polyethyleneimine (EI/PEI), attached thereon if all reactive amines are fully reacted.

A biodegradable polymer of this invention can further comprise a polymer core comprising 2 or more branching reactive sites, and two or more polymer segments each is attached to the polymer core at one of the branching reactive sites, and wherein the polymer core is alinear polymer, a branched polymer, a dendrimer, or a combination thereof. The branched polymer and dendrimer can be symmetrically (SBP) or an asymmetrically branched polymer (ABP).

A polymer core can be a cationic polymer, an anionic polymer, a charge neutral polymer, a hydrophilic polymer or a hydrophobic polymer. A polymer comprising one or more biodegradable bonds, for example, a polymerized ethyleneimine produced from cysteamine dihydrochloride, such as the ones described by Nam, et al. (J. Control Release. 220:447-455, 2015), can be suitable.

A polymer core can comprise lysine, polylysine, polyaspartic acid, polyglutamic acid, polymerized alkyleneimine, alkyldiamine, ethylenediamine, polymerized ethyleneimine (PEI), propyleneimine, propylenediamine, polymerized propyleneimine (PPI), polymerized amidoamine (PAMAM), tris(2-aminoethyl)amine (TREN), polymerized tris(2-aminoethyl)amine, polyalkylamine, polyallylamine, polyol or a combination thereof. In one example, a polymer core can comprise a lysine, an alkyl diamine, such as ethylenediamine or propylenediamaine, a tri-amine, such as tris(2-aminoethyl)amine (TREN), with two or more polymer segments disclosed herein attached thereon. In another example, a polymer core can comprise a polyol, such as a pentaerythritol or a glycerol or polyethylene glycol (PEG), with two or more lysines or polylysines attached thereon via ester bonds. In yet another example, a polymer core can comprise polyethyleneimine, polypropyleneimine, polymerized amidoamine (PAMAM), tris(2-aminoethyl)amine (TREN), polymerized tris(2-aminoethyl)amine, polyalkylamine, polyallylamine, or a combination thereof. As mentioned before, the term “polymerized ethyleneimine”, etc., refers to a polymer or a polymer segment comprising 2 or more ethyleneimine or ethyleneimine residues.

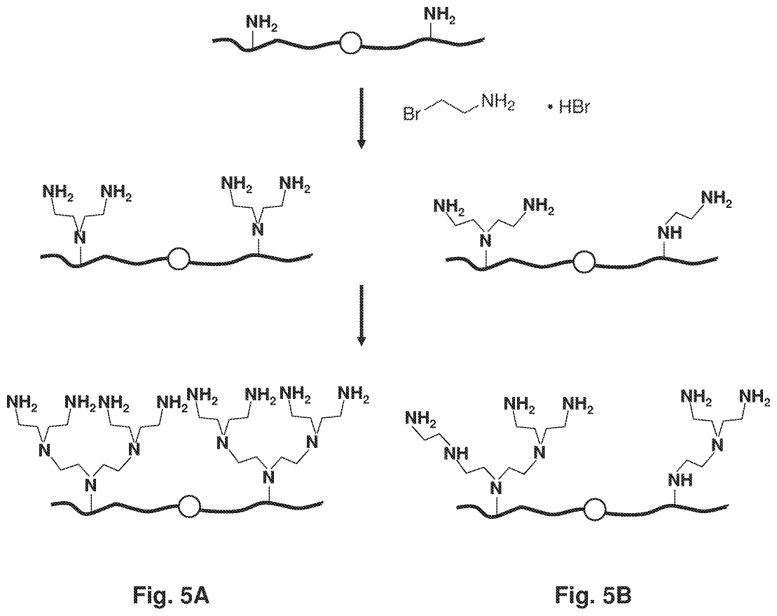

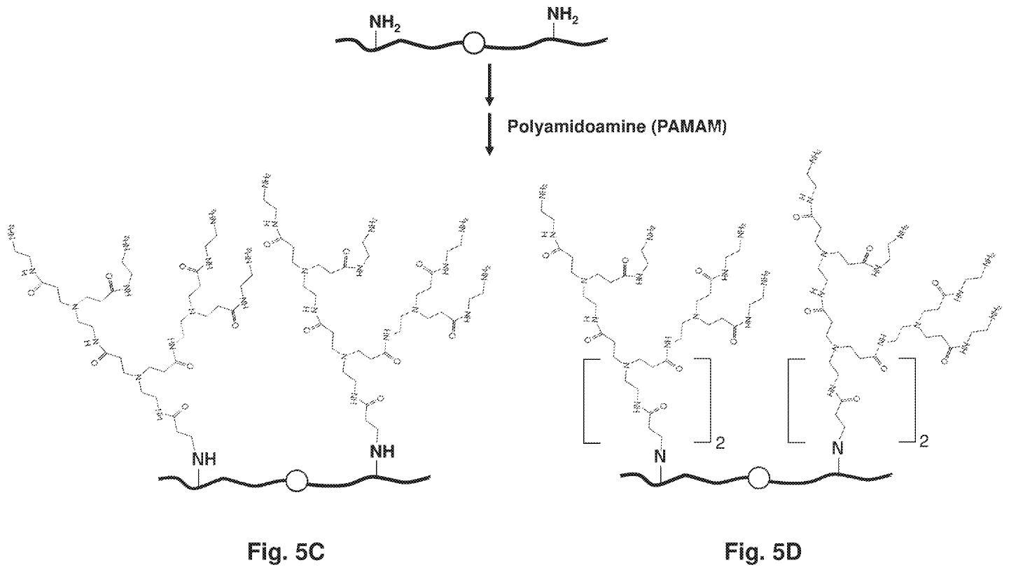

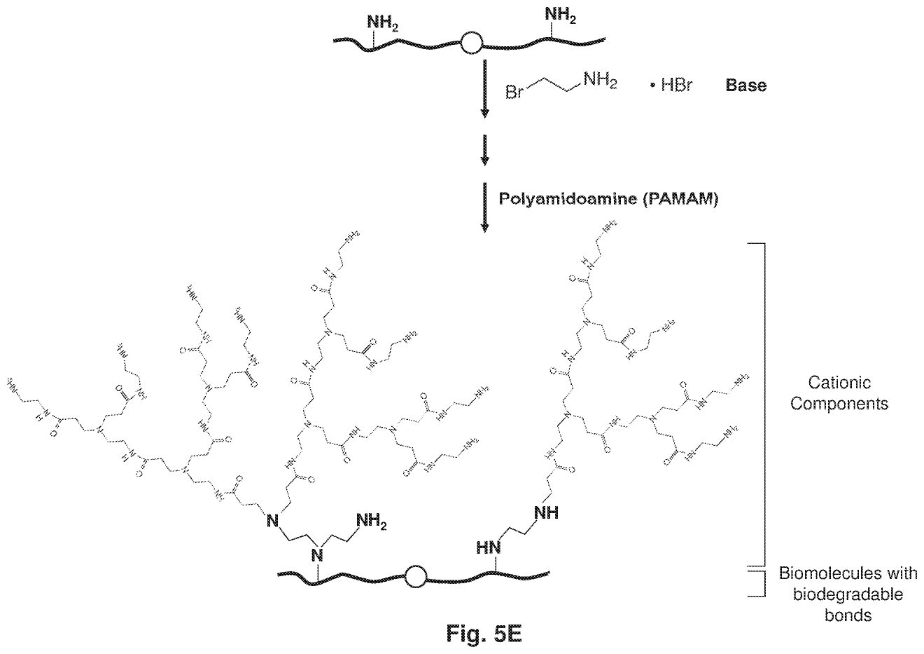

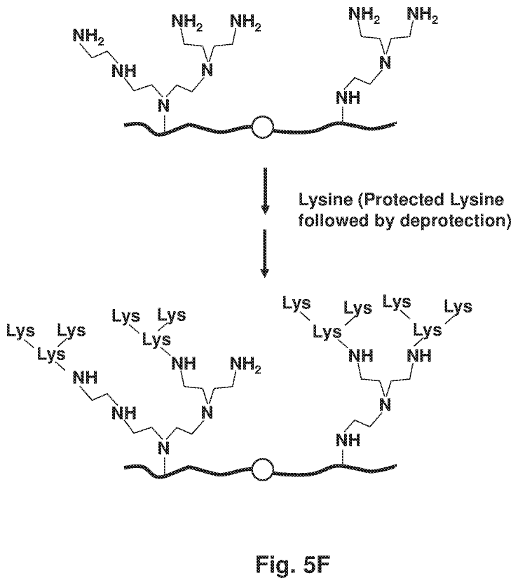

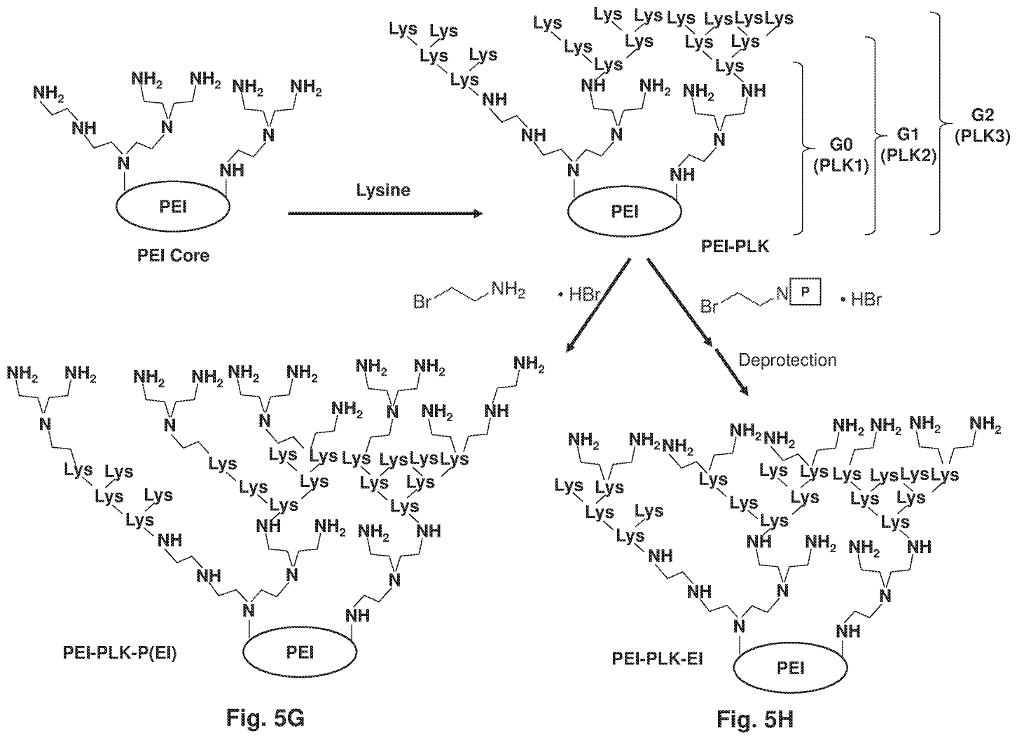

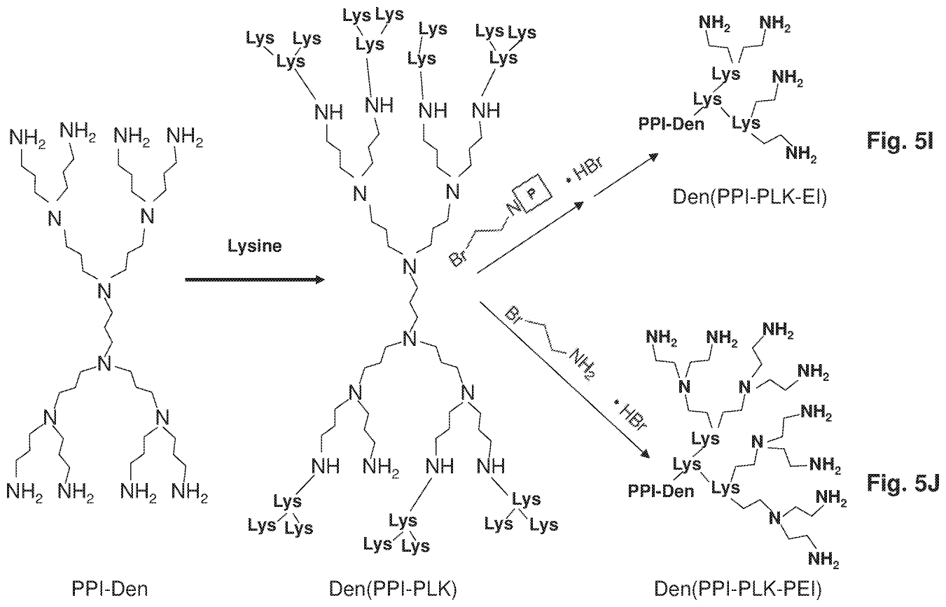

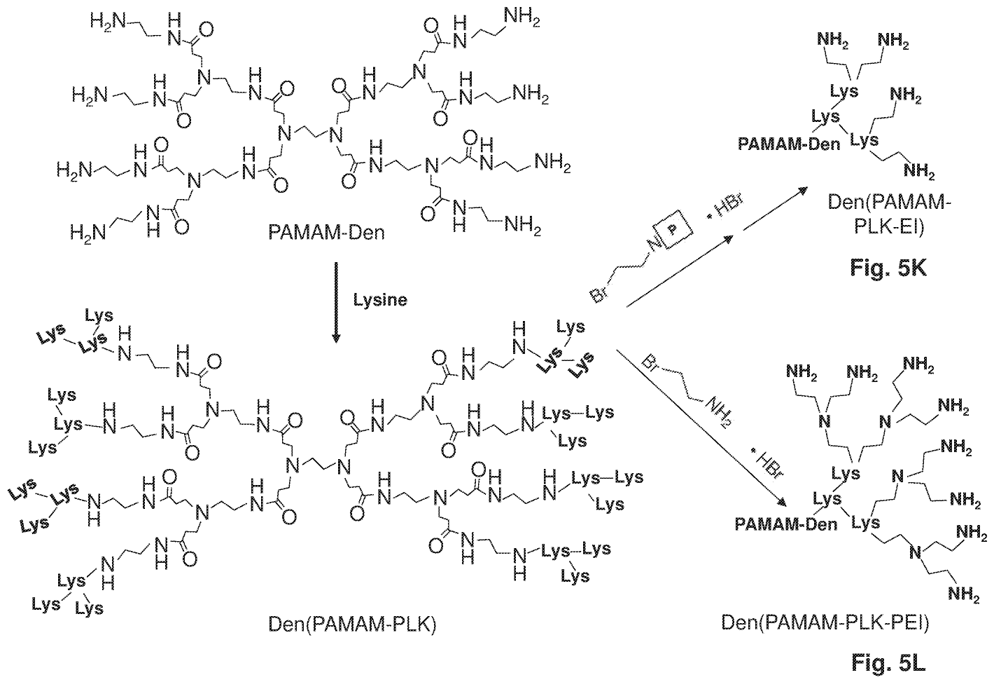

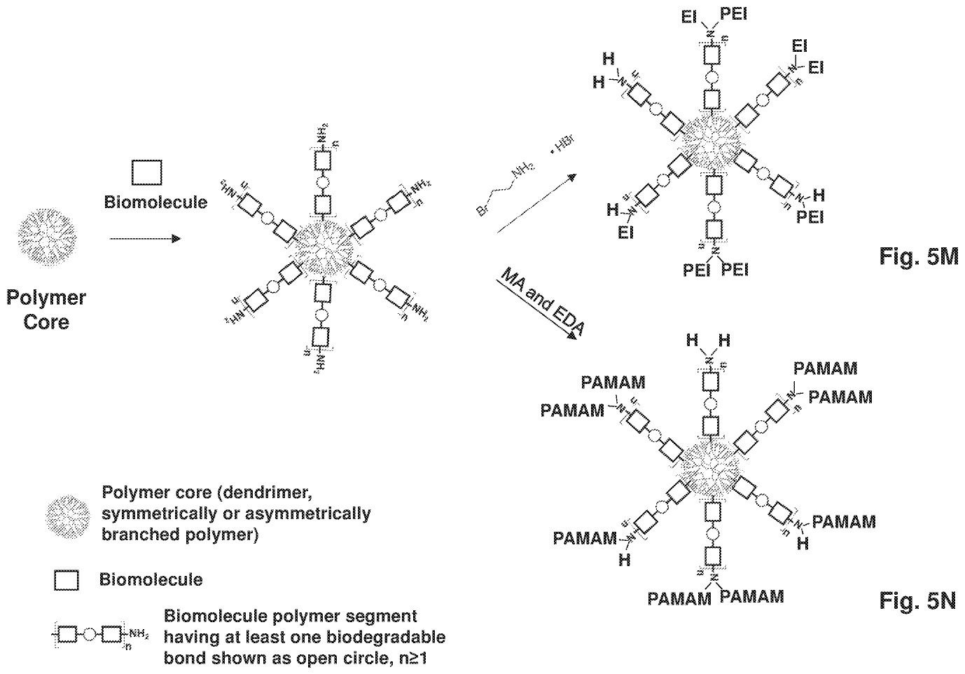

The term “branching reactive site” used herein refers to a chemical reactive group that can react with another molecule or molecules to produce a branched molecule structure, such as a symmetrically or an asymmetrically branched polymer. A polymer core can also be a biodegradable polymer core. In this structure, the biodegradable polymer can have a biodegradable polymer core and additional biodegradable polymer segments. One or more of the biodegradable polymer segments can further be modified with an amine compound, such as bromoethylamine, bromopropylamine or a combination thereof. In one example, a polymer core can be a polylysine polymer. In this structure, a biodegradable polymer can comprise a biodegradable polylysine polymer core and layers of biodegradable polylysine polymer segments and can be further modified with end amine groups. Some examples are shown in FIG. 5A-FIG. 5N.

The polymer core can comprise 2 or more branching reactive sites in one example, 3 or more branching reactive sites in another example, 4 or more branching reactive sites in yet another example, 5 or more branching reactive sites in yet another example, 6 or more branching reactive sites in a further example, 10 or more branching reactive sites in yet further example, 20 or more branching reactive sites in an even further example and 40 or more branching reactive sites in yet another example. In further examples, a polylysine having 8 lysine residues can have up to 9 reactive —NH₂ groups, a PEI core having MW 1800 can have about 31 reactive —NH₂ groups, and these reactive —NH₂ groups can be the branching reactive sites.

A polymer core of any biodegradable polymers of this invention can have a molecular weight in a range of from 40 Da to 25,000 Da in one example, 40 to 20,000 in another example, 40 to 15,000 in yet another example, 40 to 12,000 in yet another example, 40 to 10,000 in yet another example, 40 to 8,000 in yet another example, 40 to 6,000 in yet another example, 40 to 5,000 in yet another example, 40 to 4,000 in yet another example, 40 to 3,800 in yet another example, 40 to 2,000 in yet another example, 40 to 1,800 in yet another example, 40 to 1,200 in yet another example, 40 to 600 in yet another example, and 40 to 400 in a further example, all molecular weight (MW) units in Da (Dalton). In one example, a polymer core can be an ethylenediamine having 4 reactive amine groups. In another example, a polymer core can be a polyethyleneimine (PEI) having a MW in a range of from 600 to 4000 Da. In yet another example, a polymer core can be a PEI having a MW of 1,800 Da. In yet another example, a polymer core can be a PEI having a MW of 1,200 Da. In yet another example, a polymer core can be a PEI having a MW of 600 Da. A polymer core can have impact to the efficiency or delivery a bioactive agent to a biosystem, such as a cell. For example, when a polymer core is larger than 5,000 Da, it can have cytotoxicity to some cells. For those cells, a polymer core of less than 5,000 Da can be preferred.

A combination of two or more polymer cores can also be suitable. Two or more polymer cores can be inked via covalent bonds between reactive groups of the cores, between reactive groups of one core and one or more end amine groups of the other core, or between end amine groups of the cores. Reactions disclosed herein or known in common practices can be suitable.

A biodegradable polymer of this invention can comprise a formula:

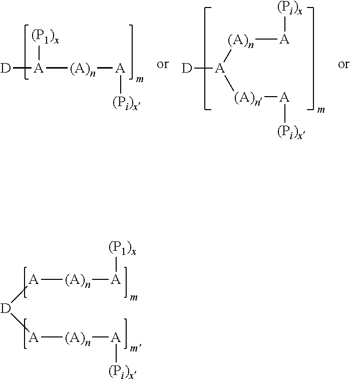

or a combination thereof,

wherein,

D is the polymer core;

m and m′ each is an integer ≥0 and m+m′≥2. P₁ through Pᵢ, x and x′, “A” are described above.

Each of the m+m′ polymer segments comprising biomolecules is attached to a polymer cord at one of the branching reactive sites of the polymer core. Some examples are schematically shown in FIG. 1F-FIG. 1I. Biomolecules can be attached to polymer core by reacting with reactive amines of the polymer core, typically with a nearly equal stoichiometry or excess amounts of biomolecules. In one example, a biodegradable polymer can comprise a branched PEI core modified with 2 to more layers such as 3 layers, of biomolecules, such as lysine and wherein at least one of amine groups of lysine residues is further modified with at least one ethylamine, propylamine, or a combination thereof. In a further example, a polymer core can comprise a polyethyleneimine (PEI) dendrimer or a branched PEI (bPEI) that have 32 reactive amine groups including primary and secondary amines. A protected lysine, such as a Boc-Lys(Boc)-OSu, can be reacted to the PEI to attach one layer of lysine onto the PEI. The resulted polymer can be deprotected and repeatedly reacted with a Boc-Lys(Boc)-OSu to attach additional layers of lysine residues onto the polymer as described later in detail in this application to produce a dendritic polymer having two or more layers of lysine residues.

Any of aforementioned cationic components can be suitable for a biodegradable polymer having a polymer core. In one example, at least one of cationic components P₁ through Pᵢ can comprise lysine residues having amine groups including end amine groups. In another example, at least one of cationic components P₁ through Pᵢ can comprise polymerized ethyleneimine, propyleneimine, or a combination thereof. In yet another example, a dendrimer having a polymer core and two or more layers of lysine residues can react with a bromoethylamine to further attach one or more ethylamines to a plurality of amine groups of the lysine residues to produce cationic components on the biodegradable polymer.

The biomolecule can comprise amino acids, such as polymerized lysine, polymerized aspartic acid, polymerized glutamic acid, or a combination thereof, or polycarbohydrate such as polysaccharides.

The polymerized amino acids, such as the aforementioned polymerized L-lysine (PLK or poly-L-lysine), polymerized D-lysine, polymerized L-glutamic acid (PLE), polymerized D-glutamic acid, polymerized L-aspartic acid, polymerized D-aspartic acid, and polycarbohydrate such as polysaccharides, or a combination thereof, forms a biodegradable polymer segment of the biodegradable polymer. The biodegradable polymer segment can be linear or branched. As used herein, the term PLK or polylysine is preferably meant polymerized modified or unmodified L-lysine, but can also include D-lysine, and the term PLE or polyglutamic acid includes polymerized modified or unmodified L-glutamic acid or D-glutamic acid, unless specifically defined.

In any biodegradable polymers of this invention, cationic components P₁ through Pᵢ can comprise the amino group of lysine residue or polylysine, additional cationic group, or a combination thereof. In one example, a biodegradable polymer comprises polylysine as a cationic component. In another example, a biodegradable polymer comprises polylysine modified with one or more ethyleneimines or ethylenediamines as cationic components. In yet another example, a biodegradable polymer comprises polylysine modified with ethylamine (EA) as a cationic component. In yet another example, a biodegradable polymer can comprise a branched PEI polymer core having 2 or more branching reactive sites each is reacted with a polylysine comprising 2 or more lysine residues (FIG. 1I) (PEI-PLK polymer). The PEI-PLK polymer can be further modified with ethyleneimine to produce a biodegradable polymer having a PEI core, 2 or more layers of lysine residues and one or more layers of ethyleneimine groups (PEI-PLK-PEI polymer). The PEI-PLK-PEI polymer can be a dendrimer, herein referred to as a Den(PEI-PLK-PEI).

In an embodiment, a biodegradable polymer can comprise a polymerized lysine, a polymerized modified lysine, a polymerized glutamic acid, a polymerized modified glutamic acid, a polymerized arginine, a polymerized modified arginine, a polymerized L-aspartic acid, polymerized modified L-aspartic acid, or a combination thereof. The biodegradable bond can comprise a peptide bond. In another embodiment, the biodegradable polymer can comprise a linear polylysine, a branched polylysine, a polyglutamic acid, a polyaspartic acid, a polyarginine, or a combination thereof.

Monomers of a polymer can be modified by practicing known methods or may be purchased commercially. Any of aforementioned modified biomolecules can be suitable.

A biodegradable polymer of this invention can comprise a polymerized sugar, disaccharide, polysaccharide, or a combination thereof. The biodegradable bond can be a glycosidic bond. A biodegradable polymer segment can comprise a disaccharide that contains, for example, glucose, fructose, or a combination thereof, sucrose, polysucrose, starch, cellulose, modified polysucrose, dextran, modified dextran, hyaluronic acid, or a combination thereof.

A biodegradable polymer of this invention can further comprise additional polymer components polymerized from other monomers that are biomolecules or non-biomolecules with biodegradable or non-biodegradable bonds, such as a polyacrylate, a polyester, a polyurethane, or the like.

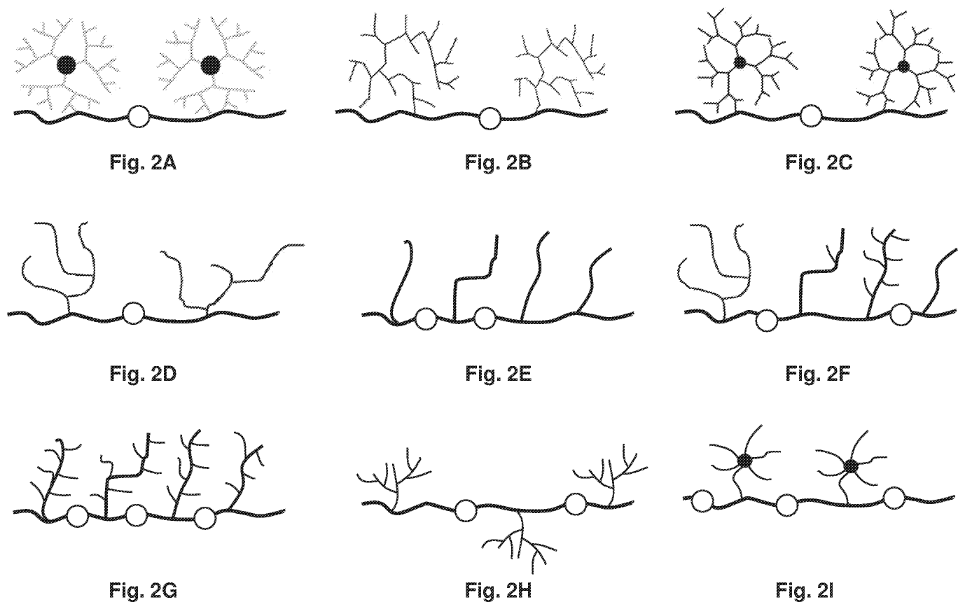

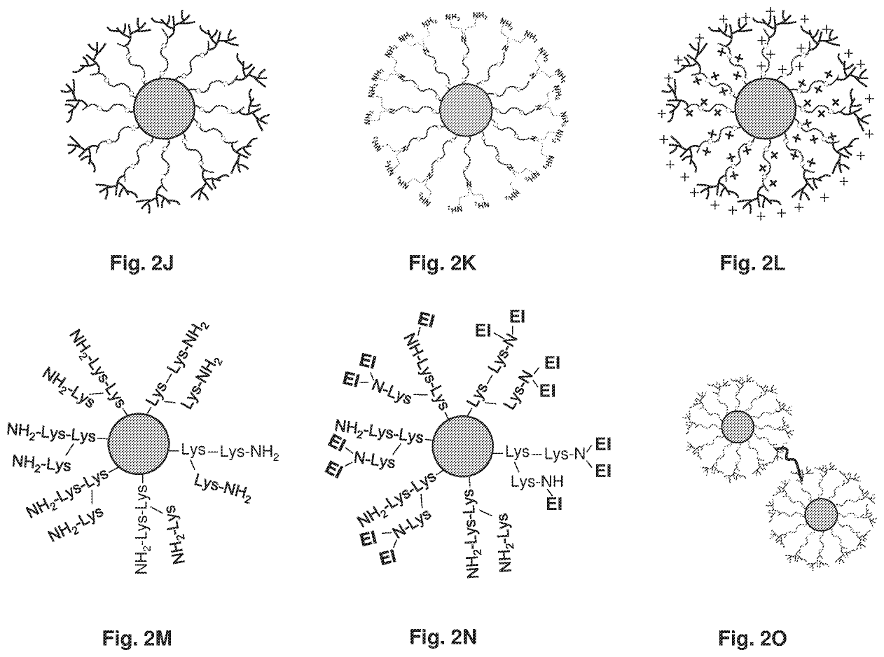

Some further examples of biodegradable polymers with linear polymer backbones are schematically shown in FIG. 2A-FIG. 2I, wherein cationic components can be: FIG. 2A, symmetrically branched polymers, including dendrimers; FIG. 2B, asymmetrically randomly branched polymers; FIG. 2C, asymmetrically regularly branched polymers; FIG. 2D, branched polymers; FIG. 2E, linear polymers; FIG. 2F, branched and/or linear polymers; FIG. 2G, comb polymers; FIG. 2H, dendronized polymers; and FIG. 2I, star-branched polymers. Some further examples of biodegradable polymers with dendritic polymer cores are schematically shown in FIG. 2J-FIG. 2O, wherein cationic components can be: (J) attached to ends of biodegradable polymer segments; (K) ethyleneimine (EI) groups attached to end amine groups of biodegradable polymer segments; (L) multiple cationic components attached to a polymer core; (M) polylysines attached to a polymer core; (N) polylysines attached to a polymer core and further modified with one or more ethylamine end amine groups and (O) attached to polymer cores in a polymer dimmer. A polymer dimmer can be attached together via one or more covalent bonds from one or more end amine groups, such as depicted in FIG. 2O, or via one or more covalent bonds from polymer cores. When two or more biodegradable polymer segments are attached to a polymer core, each of the biodegradable polymer segments can comprise one or more cationic components, so that two or more cationic components in the same or different biodegradable polymer segments can be separated by at least one biodegradable bond in the biodegradable polymer such as those shown in FIG. 2J-FIG. 2O and FIG. 5M-FIG. 5N. When biodegradable bonds are cleaved, these cationic components can be released from the polymer. As mentioned above, a biodegradable polymer segment can be linear or branched. Each open circle in the figures represents one or more biodegradable bonds.

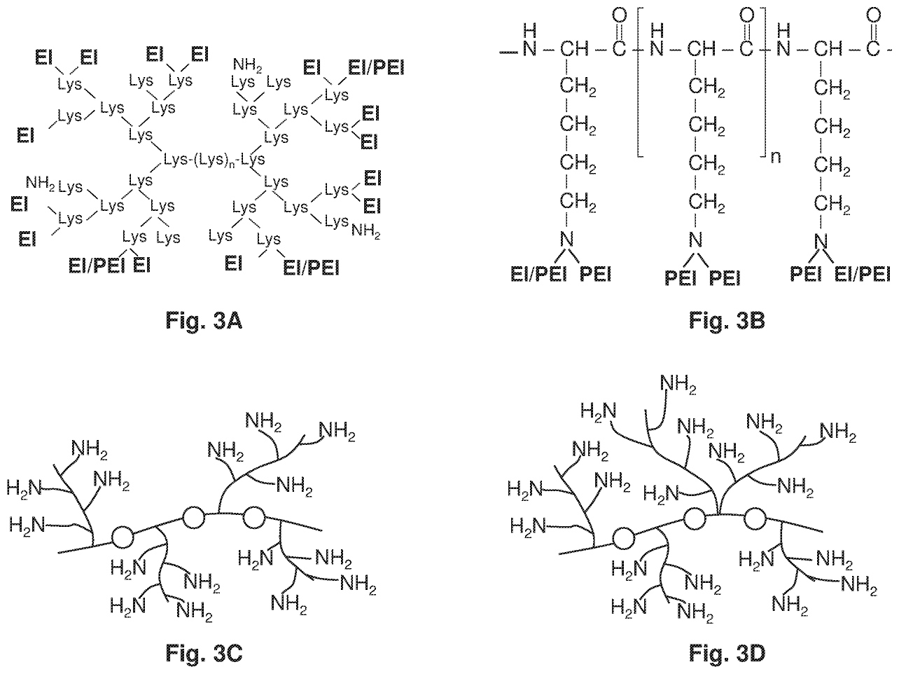

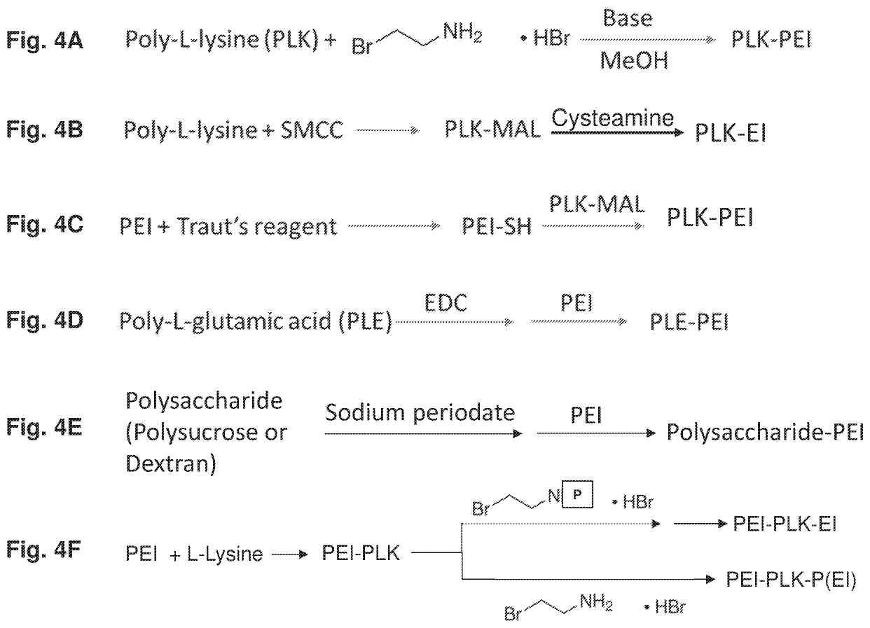

Examples of biodegradable polymers comprising branched or linear poly-L-lysine and polyethyleneimine (PEI) are shown in FIG. 3A-FIG. 3B, wherein n can be in a range of from 0 to 10,000 (FIG. 3B). An example of a polymer having branched cationic components with —NH₂ end amine groups is shown in FIG. 3C. In a further example, two PEI segments can be attached to the same lysine in a polymeric backbone (FIG. 3D). Each of the cationic components, including the aforementioned P₁ through Pᵢ, can be the same or different. For example, each of the cationic components can be different in the number of repeating units, length of polymer chain, degree of branching, monomer composition, polymer formulae, modification, or a combination thereof. Each of the cationic components can also be independently modified before, during or post polymerization.

A cationic component can comprise functional groups, monomers or polymers, such as, an amine group, a compound comprising an amine group or a combination thereof. Each of the cationic components can comprise one or more amine groups selected from a primary amine, a secondary amine, a tertiary amine, or a combination thereof, one or more cationic amino acids selected from lysine, polylysine, arginine, polyarginine, histidine, polyhistidine or a combination thereof, polymerized alkyleneimine, polymerized ethyleneimine (PEI), polymerized propyleneimine (PPI), polymerized amidoamine (PAMAM), tris(2-aminoethyl)amine (TREN), polymerized tris(2-aminoethyl)amine, polyalkylamine, polyallylamine, or a combination thereof.

Some examples of reactions for producing a biodegradable polymer of this invention are shown in FIG. 4A-FIG. 4F. For example, a polyethyleneimine (PEI) can be grown from a polylysine (PLK): a poly-L-lysine can be reacted with ethyleneimine bromide to attach and grow PEI onto the poly-L-lysine via polycondensation growth. Polymer properties, such as molecular weight, size of polymer chain, degree of branching, or a combination thereof, can be controlled as a design choice, for example, by altering reagents and or reaction conditions. The reaction is schematically shown below and also in FIG. 4A:
Poly-L-Lysine (PLK)+BrCH₂CH₂NH₂—HBr+Base→PLK-PEI.

A base can be used for the reaction in FIG. 4A. In one example, a base can be selected from N,N-diisopropylethylamine (DIPEA), triethylamine, carbonate (sodium or potassium), hydroxide (sodium or potassium), other suitable bases, or a workable combination thereof. Although specific compounds are listed here, the reactions, reactants and reagents are not limited to those listed herein. Any reactions, reagents that can produce the desired product specified herein can be suitable.

In one example, a polylysine can react with an SMCC to produce a SMCC modified polylysine (PLK-MAL) (FIG. 4B). A preformed polymerized ethyleneimine (PEI) can first react with a Traut's agent to produce a modified PEI-SH and then reacts with a PLK-MAL to attach the PEI onto the biodegradable polymer backbone to produce a biodegradable polymer of this invention, PLK-PEI (FIG. 4C). SMCC is an amine to sulfhydryl linker that contains a reactive NHS-ester group and on the other end of the molecule, a reactive maleimide group, hence, the NHS-ester reacts with an amine group of polylysine and binds thereto to expose a maleimide group. SMCC is available commercially. The reactions can be schematically shown below:
Poly-L-Lysine+SMCC→PLK-MAL
PEI+Traut's reagent→PEI-SH
PEI-SH+PLK-MAL→PLK-PEI.

The PLK-MAL can also be reacted with cysteamine (H₂NCH₂CH₂SH) to produce a biodegradable polylysine polymer having a thio-bond and multiple end amine groups and can be referred to as a cysteamine modified polylysine with multiple ethylamine (EA) (or ethyleneimine, EI) groups. A cysteamine modified polylysine can be further reacted with bromoethyleneamine or bromopropyleneamine to produce a polylysine polymer having PEI or PPI cationic components. The reactions are schematically shown below (FIG. 4B-FIG. 4C) (not all reaction reagents are shown for brevity):
PLK-MAL+H₂NCH₂CH₂SH→PLK-EI (Multiple EI on PLK)
PLK-EI (Multiple EI on PLK)→PLK-PEI or PLK-PPI.

In another example, a biopolymer, such as a polylysine can be a biodegradable polymer segment and can react with a chloroethyleneamine, a bromoethyleneamine, an iodoethyleneamine, or a combination thereof, to grow polyethyleneimine (PEI) to produce a biodegradable polymer of this invention. Chloropropyleneamine, bromopropyleneamine, iodopropyleneamine, or a combination thereof, can be used to grow polypropyleneimine (PPI) onto a biodegradable polymer segment. Poly-L-glutamic acid (PLE) can react with PEI to produce a biodegradable polymer of this invention that comprises biodegradable glutamic acid peptide bonds and PEI cationic components (FIG. 4D). Poly-L-glutamic acid (PLE) can also react with a protected ethyleneimine bromide to produce a biodegradable polymer of this invention that have biodegradable glutamic acid peptide bonds and multiple EI end amine groups. Polyaspartic acid (PLD) can be reacted with EDC similar to that of poly-L-glutamic acid to produce a biodegradable polymer of this invention that have biodegradable aspartic acid peptide bonds and PEI or EI cationic components. Polysaccharide, polysucrose or dextran can react to PEI to produce a biodegradable polymer of this invention that have biodegradable polysaccharide and PEI cationic components (FIG. 4E). In another example, a polymerized ethyleneimine (PEI) can react with a plurality of amino acids, such as lysine, to result in a dendritic polymer intermediate that has a PEI core and one layer of lysine residues (G0), two layers of lysine residues (G1) or more (G2, G3 and so on). Some examples of a dendrimer having a polymerized lysine (PLK) over a PEI core, herein referred to as Den(PEI-PLK), such as a dendrimer of a PEI core and two layers of lysine residues, herein referred to as Den(PEI-Lys-Lys), are shown below and also in FIG. 4F. Protected L-Lysine such as Boc-Lys(Boc)-OSu can be used.

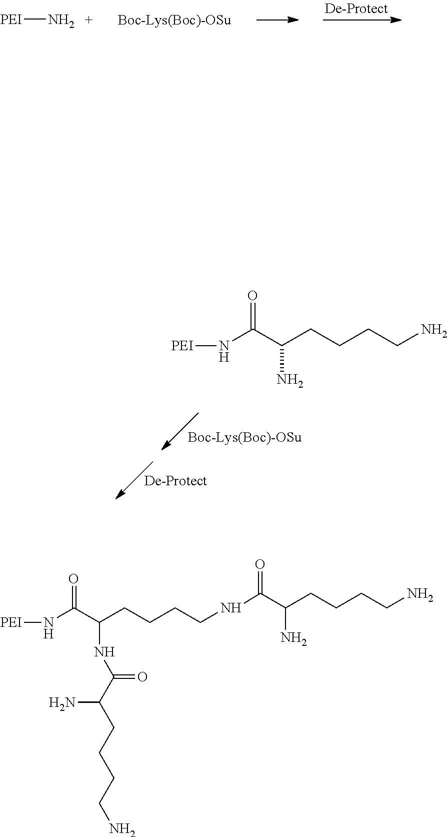

A Den(PEI-PLK) dendrimer can be further modified by reacting with protected or unprotected amine (EI or EI-P), such as bromoethylamine or Boc-bromoethylamine, respectively, to produce a biodegradable polymer of this invention that have biodegradable lysine-lysine peptide bonds and ethylamine cationic components. Multiple layers, such as G1, G2 or more, of lysine residues can be preferred. A biodegradable polymer of this invention can comprise 2 or more layers of lysine residues, wherein the lysine-lysine peptide bond is biodegradable such as by enzymes in vivo or in vitro. At least one of the lysine end amine groups can be modified to further have one or more amine cationic groups, such as ethyleneimine or polyethyleneimine (FIG. 5A-FIG. 5N). The amount of ethyleneimine or the number of layers of ethyleneimine in a biodegradable polymer can be adjusted to provide optimized property for interaction with the bioactive agent or agents. When a polylysine (PLK) is used, the —NH₂ group or a charged form such as —NH₃⁺ from lysine can function as a cationic component P₁ through Pᵢ, such as those shown in FIG. 1A-FIG. 1I.

The terms or abbreviations used herein are: MeOH, methanol; SMCC, succinimidyl-trans-4-(N-maleimidylmethyl)cyclohexane-1-carboxylate; MAL, maleimide; Traut's reagent, 2-iminothiolane; EDC, N-ethyl-N′-(3-dimethylaminopropyl)carbodiimide hydrochloride. The group P in ethyleneimine (EI-P) shown in FIG. 4F represents a protection group. Examples of a protection group can include t-butyloxycarbonyl (tert-Butyloxycarbonyl, t-BOC) protecting group, and OSu (N-Hydroxysuccinimide (NHS) activated ester), or a combination thereof. Other reagents and conditions that can be used for amide bond formation are described in Valeur et al, Chem. Soc. Rev., 2009, 38, 606-631. Other protection groups can include those described in Greene's Protective Groups in Organic Synthesis (Edition 5) by Peter Wuts, Wiley, 2014.

At least one of cationic components of a biodegradable polymer of this invention can comprise at least one end amine group selected from —N(CH₂)NH₂, —N(CH₂)₂NH₂, —N(CH₂)₃NH₂, —N(CH₂)N⁺H₃, —N(CH₂)₂N⁺H₃, —N(CH₂)₃N⁺H₃, or a combination thereof. In one example, a cationic component can comprise polymerized ethyleneimine (PEI) (FIG. 5A-FIG. 5B) or polymerized amidoamine (PAMAM) (FIG. 5C-FIG. 5D). In another example, cationic components can comprise polymerized ethyleneimine (PEI), polymerized propyleneimine (PPI), polymerized amidoamine (PAMAM), tris(2-aminoethyl)amine (TREN), polymerized tris(2-aminoethyl)amine, polyalkylamine, polyallylamine, or a combination thereof. In a further example of a biodegradable polymer of this invention, some or each of the cationic components can comprise polymerized ethyleneimine (PEI), polymerized propyleneimine, and/or polymerized amidoamine (PAMAM). In yet another example, a biodegradable polymer of this invention can comprise at least one ethyleneimine (EI) monomeric unit or moiety attached to a biomolecule of a backbone. That single EI moiety can be used as a cationic component or can serve as a starting point for constructing a PEI for subsequent monomer attachment of one or a plurality of, for example, ethyleneimine(s), amidoamine(s), or a combination thereof, forming a cationic component comprising, for example, multiple EI/PEI (FIG. 5A-FIG. 5B), or a EI/PEI that is further modified with polyamidoamine (PAMAM) (FIG. 5E) using practicing methods known in the art. A biodegradable polymer of this invention can comprise two or more such cationic components separated by at least one biodegradable bonds (FIG. 5E). The PEI cationic components of a biodegradable polymer can also be modified with lysine residues or polylysine (FIG. 5F).

A biodegradable polymer of this invention can further comprise a polymer core. A polymer core can be selected from a dendrimer, a symmetrically branched polymer, or an asymmetrically branched polymer. For example, a polymer core can be a polyethyleneimine (PEI) and can be modified with biomolecules, for example, one or more lysine residues or polylysine (PLK) having lysine-lysine peptide bonds. A resulted polymer can be further modified with additional one or more layers of ethyleneimine (EI/PEI) (FIG. 5G-5H). In one example, a polymerized propyleneimine (PPI) dendrimer having at least 8 —NH₂ terminals can be modified with two or more layers of lysine residues and further modified with protected ethylenediamine to form a dendritic biodegradable polymer having a dendrimer core-polylysine and at least one layer of ethyleneimine (EI), herein referred to as Den(PPI-PLK-EI) (FIG. 5I). The polymer can also be modified with ethyleneimine (EI)/ethylene diamine to form two or more layers of polyethyleneimine (PEI), herein referred to as Den(PPI-PLK-PEI) (FIG. 5J). In another example, a polyamidoamine (PAMAM) dendrimer can be modified with two or more layers of lysine residues and further modified with a protected ethyleneimine (p-EI, such as Boc-bromoethylamine) or bromoethylamine (EI) to produce a biodegradable polymer having two or more layers of lysine residues and at least one layer of ethyleneimine as a cationic component, herein referred to as Den(PAMAM-PLK-EI) (FIG. 5K) or two or more layers of ethyleneimine (PEI) as a cationic component, herein referred to as Den(PAMAM-PLK-PEI) (FIG. 5L). Schematic outlines of examples of polymer cores modified with biomolecule polymer segments each having at least one biodegradable bond and further modified with one or more cationic components such as ethyleneimine (EI) or polyethyleneimine (PEI), or polyamidoamine (PAMAM) are shown in FIG. 5M-FIG. 5N. Each open circle represents one or more biodegradable bonds. When biodegradable bonds are cleaved, these cationic components can be released from the polymer. Although only two layers of lysine and 1-2 layers of ethyleneimine are shown in figures, a biodegradable polymer can comprise two or more layers of lysine residues and one or more layers of ethyleneimine or propyleneamine resulting in a plurality of end amine groups. A biomolecule can comprise amino acids, such as polymerized lysine, polymerized aspartic acid, polymerized glutamic acid, or a combination thereof, or polycarbohydrate such as polysaccharides. For simplicity, not all bonds or components are shown. MA is methylacrylate. EDA is ethylene diamine.

A biodegradable polymer can also be polymerized from monomers that comprise non-biomolecule monomers or forming non-biodegradable bonds with a proviso that the biodegradable polymer comprises two or more cationic components separated by at least one biodegradable bond.

Each of the cationic components can be independently alinear polymer, a branched polymer (symmetric SBA and/or asymmetric ABP), a hyperbranched polymer, a graft polymer, a star polymer, a block polymer, a dendrimer, or a combination thereof. The term “polymer” or “copolymer” used herein and throughout this application refers to polymer polymerized from the same monomer (homopolymer), two or more different monomers (copolymer), or a combination thereof, unless specifically specified. Also as mentioned herein, cationic components can be the same or different.

In one embodiment, cationic components, such as a polymerized polyethyleneimine (PEI), a propylethyleneimine (PPI), or a combination thereof, can comprise primary amine (1°), secondary amine (2°) and tertiary amine (3°) groups which can be in a primary:secondary:tertiary ratio of design choice. As mentioned hereafter, a biodegradable polymer of this invention can have an advantage of easily being adjusted for its amine contents, primary:secondary:tertiary amine ratios and electric cationic charges to suit various uses.

A biodegradable polymer of this invention can further comprise at least one hydrocarbon chain having in a range of from 3 to 30 carbon atoms (C3-C30), wherein the hydrocarbon chain is covalently attached to the biodegradable polymer. The hydrocarbon chain can comprise saturated or unsaturated linear alkyl groups, cyclic alkyl groups, one or more allyl groups, one or more aromatic carbon groups, or a combination thereof. The hydrocarbon chain can be a reacted unsaturated fatty acid, a saturated fatty acid, an epoxide derivative of said unsaturated fatty acid, an epoxide derivative of said saturated fatty acid, or a combination thereof.

In any of biodegradable polymers of this invention, a hydrocarbon chain can be attached to one or more of the biomolecules, one or more of the cationic components, or a combination thereof. A hydrocarbon chain can be attached to one of the biomolecules in one example, attached to one of the cationic components in another example, or a combination thereof. A hydrocarbon chain can be attached to an amine group of the biomolecules, such as an amine group of a lysine residue or a polylysine in some examples. The hydrocarbon chain can be attached to an amine group of one or more of the cationic components, for example, a free amine group of lysine residue, polylysine, alkyleneimine, polymerized alkyleneimine, ethyleneimine, polymerized ethyleneimine (PEI), propyleneimine, polymerized propyleneimine (PPI), polymerized amidoamine (PAMAM), tris(2-aminoethyl)amine (TREN), polymerized tris(2-aminoethyl)amine, polyalkylamine, polyallylamine or a combination thereof. In another example, a hydrocarbon chain can be attached to one of the amine groups of the biodegradable polymer selected from —N(CH₂)NH₂, —N(CH₂)₂NH₂, —N(CH₂)₃NH₂, or a combination thereof.

A biodegradable polymer of this invention can comprise two or more cationic components, a branched or linear polylysine having 2 or more lysine residues and one or more lysine-lysine biodegradable peptide bonds, and at least one hydrocarbon chain having in a range of from 3 to 30 carbon atoms (C3-C30), wherein the cationic components are attached to the lysine residues covalently and separated by at least one of the lysine-lysine biodegradable peptide bonds, and wherein the hydrocarbon chain is covalently attached to the polylysine. A biodegradable polymer of this invention can also comprise two or more cationic components, a branched or linear polylysine having 2 or more lysine residues and one or more lysine-lysine biodegradable peptide bonds, and at least one hydrocarbon chain having in a range of from 3 to 30 carbon atoms (C3-C30), wherein the cationic components are attached to the lysine residues covalently and separated by at least one of the lysine-lysine biodegradable peptide bonds, at least one of the cationic components comprises one or more end amine groups selected from —N(CH₂)NH₂, —N(CH₂)₂NH₂, —N(CH₂)₃NH₂, or a combination thereof, and wherein the hydrocarbon chain is attached to at least one of the end amine groups covalently. In a further example, a biodegradable polymer of this invention can comprise two or more cationic components, a branched or linear polylysine having 2 or more lysine residues and one or more lysine-lysine biodegradable peptide bonds, and two or more hydrocarbon chains each having in a range of from 3 to 30 carbon atoms (C3-C30), wherein the cationic components are attached to the lysine residues covalently and separated by at least one of the lysine-lysine biodegradable peptide bonds, at least one of the cationic components comprises one or more end amine groups selected from —N(CH₂)NH₂, —N(CH₂)₂NH₂, —N(CH₂)₃NH₂, or a combination thereof, and wherein at least one of the hydrocarbon chains is attached to the end amine groups covalently and at least another of the hydrocarbon chains is covalently attached to the polylysine. In yet another example, at least two of hydrocarbon chains are attached to the end amine groups of the biodegradable polymer. In yet a further example, at least two of hydrocarbon chains are attached to the polylysine of the biodegradable polymer. Any of cationic components of this invention can be suitable.

A hydrocarbon chain can be a reacted propionic acid, butyric acid, valeric acid, caproic acid, enanthic acid, caprylic acid, pelargonic acid, capric acid, undecylic acid, lauric acid, tridecylic acid, myristic acid, pentadecylic acid, palmitic acid, margaric acid, stearic acid, nonadecylic acid, arachidic acid, heneicosylic acid, behenic acid, tricosylic acid, lignoceric acid, pentacosylic acid, cerotic acid, heptacosylic acid, montanic acid, nonacosylic acid, melissic acid, henatriacontylic acid, lacceroic acid, psyllic acid, geddic acid, ceroplastic acid, hexatriacontylic acid, heptatriacontanoic acid, octatriacontanoic acid, α-linolenic acid, stearidonic acid, eicosapentaenoic acid, docosahexaenoic acid, linoleic acid, linolelaidic acid, γ-linolenic acid, dihomo-γ-linolenic acid, arachidonic acid, docosatetraenoic acid, palmitoleic acid, vaccenic acid, paullinic acid, oleic acid, elaidic acid, gondoic acid, erucic acid, nervonic acid, mead acid, an isomer derivative thereof, an epoxide derivative thereof, or a combination thereof. The term “reacted” used herein refers to an acid or an epoxy group that is reacted with an amine group of a biodegradable polymer attaching the hydrocarbon chain to the polymer. As used herein, isomers refer to molecules that share the same chemical formula but have their atoms connected differently or arranged differently in space, including structural isomers having respective atoms bonded together in different orders, geometric isomers having atoms bonded in the same order, but differ in the configuration around the bonds, such as cis- or trans-isomers and enantiomers having the same chemical structure but differ in three-dimensional arrangements of atoms around asymmetric carbon, such that they are mirror images of one another.

In some examples, a C18 saturated or unsaturated hydrocarbon chain in acid form can be reacted to amine group of a biodegradable polymer as shown below. As used herein, an amine group can be from a lysine residue or an end amine group.

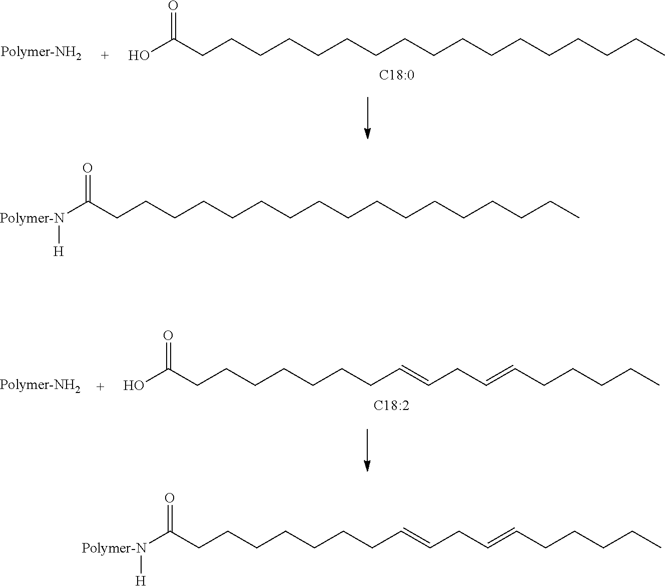

In other examples, a C18 saturated or unsaturated hydrocarbon chain in epoxy form can be reacted to amine group of a biodegradable polymer as shown below.

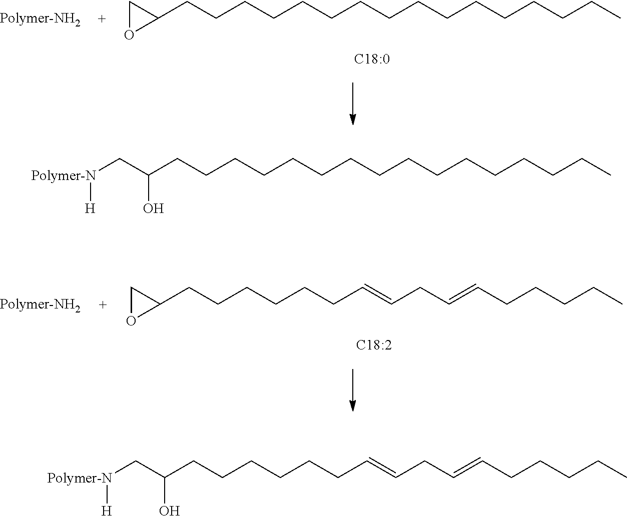

The cationic components of the biodegradable polymer of this invention can each comprise lysine, polylysine, or a combination thereof. The hydrocarbon chain can be attached to an amine group of the lysine or polylysine. In one example, a biodegradable polymer can comprise 4 or more polymerized lysine (polylysine) and at least one hydrocarbon chain attached to an amine group of the polylysine. In another example, a biodegradable dendritic polymer can comprise a branched or dendritic polyethyleneimine (PEI) polymer core and two or more layers of polylysine attached thereon, and at least one hydrocarbon chain attached to an amine group of the polylysine, an amine group of the PEI, or a combination thereof.

In a further example, biomolecules of a biodegradable polymer of this invention can consist of lysine and polylysine and the cationic components each can consist of lysine and polylysine. One or more hydrocarbon chains can be attached to an amine group of the lysine, polylysine or a combination thereof.

Some examples of a hydrocarbon chain attached to a lysine or a polylysine, one or more end amine groups of EI/PEI are shown here. It is understood various hydrocarbon chains of C3 through C30 can be attached to a biodegradable polymer via any suitable reactions by practicing known methods, including, but not limited those exemplified herein.

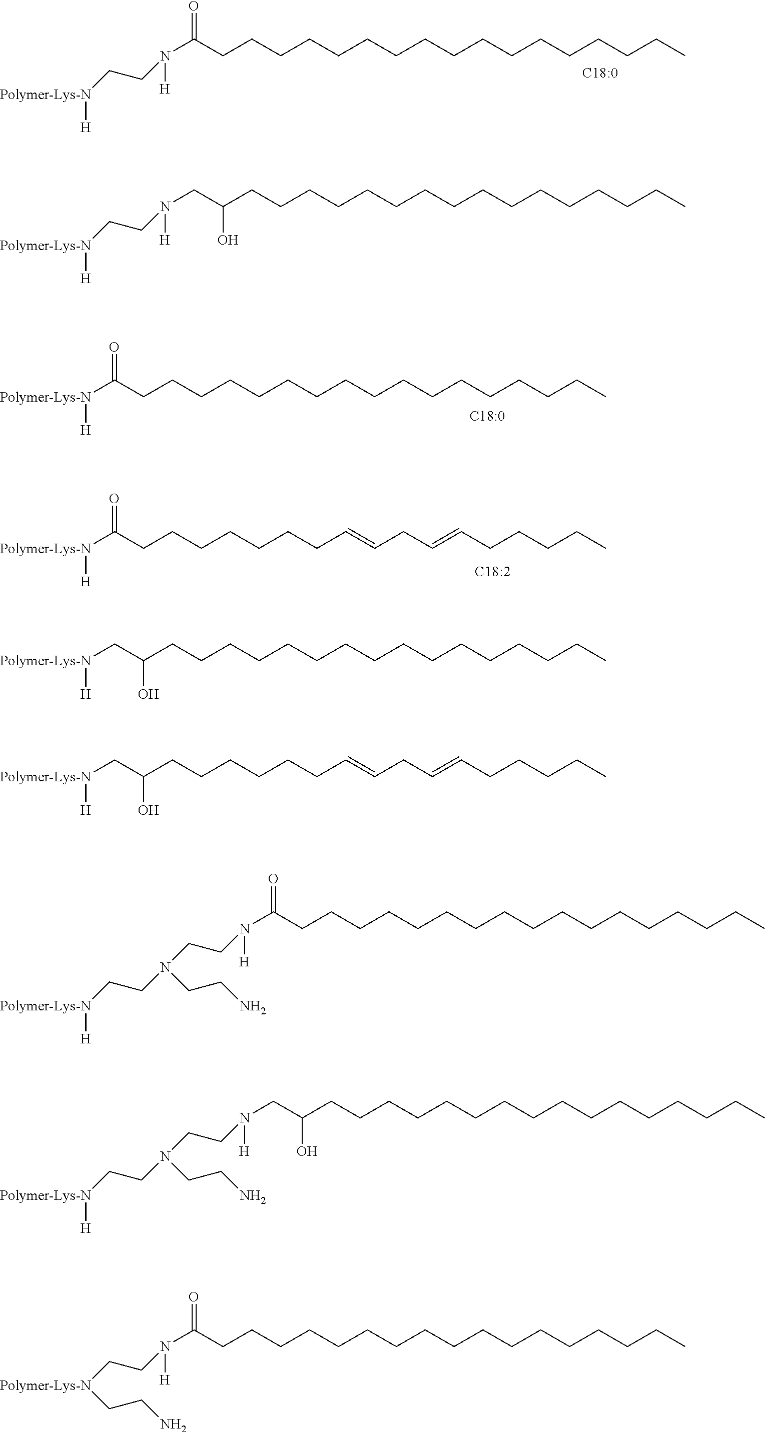

In further examples, biomolecules of biodegradable polymers of this invention can consist of lysine and polylysine and the cationic components can comprise ethyleneimine, polymerized ethyleneimine (PEI), propyleneimine, polymerized propyleneimine (PPI), polymerized amidoamine (PAMAM), or a combination thereof.

This invention is also directed to a bioactive composition comprising a biodegradable polymer and at least one bioactive agent. Any biodegradable polymers of this invention disclosed above and hereafter can be suitable. Any combination of biodegradable polymers of this invention disclosed above and hereafter can also be suitable.

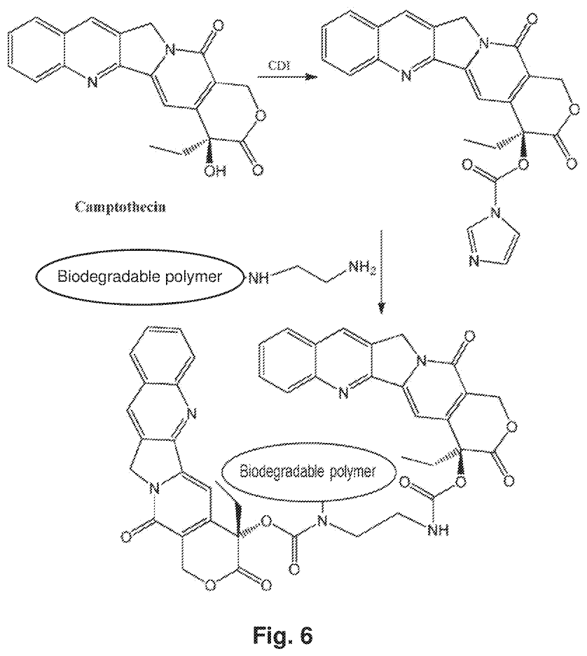

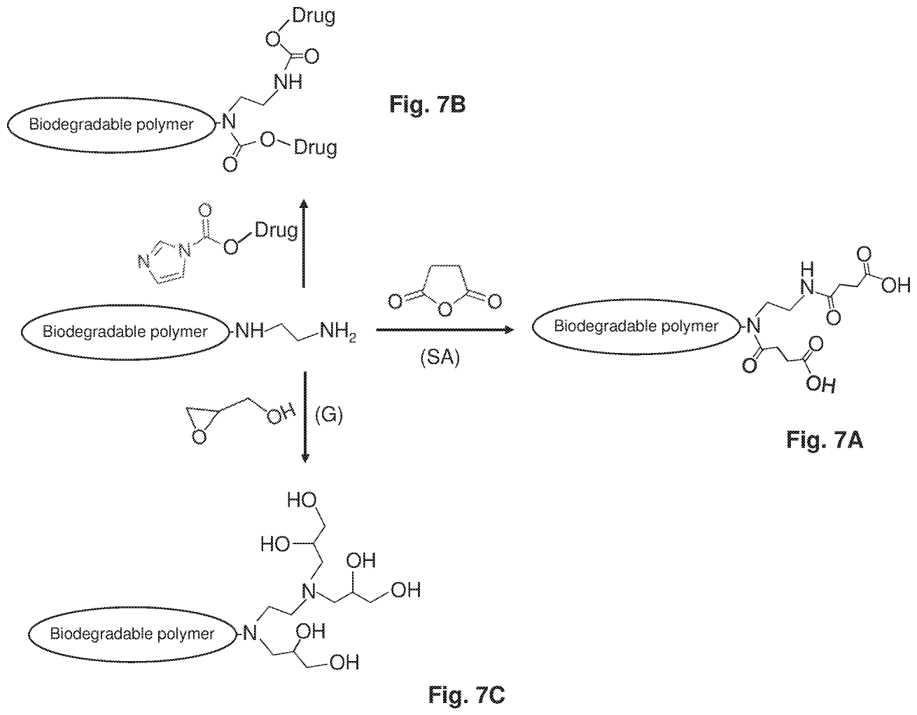

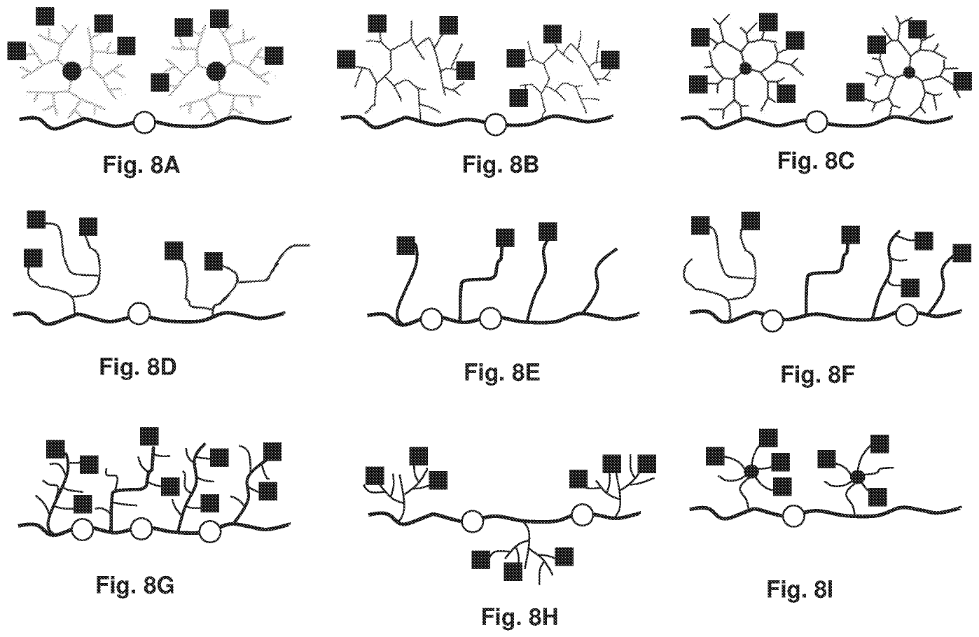

A biodegradable polymer and a bioactive agent can be linked with one or more covalent bonds or one or more non-covalent linkages. A biodegradable polymer can be linked to one or more drug molecules or additional functional groups. In one example, a small molecule drug, such as one or more camptothecin molecules can be reacted to a linking molecule, for example, a CDI (carbonyl diimidazole) and then reacted with one or more of amine groups of a biodegradable polymer to be attached thereon (FIG. 6). In another example, a biodegradable polymer can attach one or more functional groups (FIG. 7A and FIG. 7C) or a specific drug molecule (FIG. 7B). A linker, such as a CDI, can be utilized. Additional examples of biodegradable polymers and bioactive agents are shown in FIG. 8A-FIG. 11F.

For a bioactive composition of this invention, a bioactive agent can be an RNA, an mRNA, an RNAi, a siRNA, an microRNA, an oligonucleotide, a DNA, an oligodeoxynucleotide, a protein, a peptide, an antibody, a fragment of an antibody, a chemical compound, a small molecule drug, a chemotherapy drug, or a combination thereof. Any of the bioactive agent disclosed above and hereafter can be suitable. In one example, a bioactive agent is a first oligodeoxynucleotide that is attached to a biodegradable polymer through a non-covalent linkage.

The term “DNA” or “DNAs” used herein can include double stranded DNA, single stranded DNA, triplex DNA, a Spiegelmer, an aptamer, a modified double stranded DNA, a modified single stranded DNA, an oligodeoxynucleotide or a modified oligodeoxynucleotide, unless specifically defined. The term “RNA” or “RNAs” refers to an oligoribonucleotide, a coding RNA, a non-coding RNA, an antisense RNA, a modified or capped RNA, a single stranded RNA, a double stranded RNA, a modified RNA, or a combination thereof unless specifically defined.

A bioactive agent can have a molecular weight in a range of from about 10 to 1,000,000 in one example, 100 to 500,000 in another example, 100 to 200,000 in yet another example, 500 to 200,000 in yet another example, 1,000 to 200,000 in yet another example, 5,000 to 200,000 in yet another example, 10,000 to 200,000 in yet another example, 15,000 to 200,000 in yet another example, 20,000 to 200,000 in yet another example, and 25,000 to 200,000 in yet another example. A bioactive agent can also have a molecular weight in a range of from about 100 to 100,000 in one example, 100 to 75,000 in yet another example, 100 to 50,000 in yet another example, 100 to 30,000 in yet another example, and 100 to 25,000 in yet another example.

A bioactive agent can be a small molecule drug. In one example, a small molecule drug, such as one or more camptothecin molecules can be attached to a biodegradable polymer via a covalent bond (FIG. 6) with or without a linker. In another example, drug molecules or one or more functional groups such as hydroxyl functional groups, can be attached to a biodegradable polymer (FIG. 7A-FIG. 7C). Multiple bioactive agents can be attached to a biodegradable polymer.

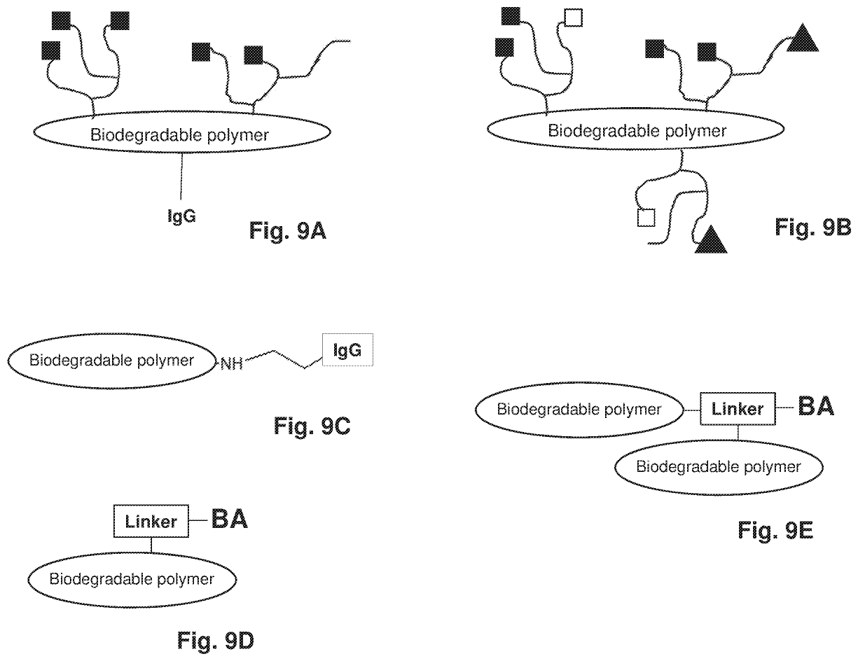

A bioactive composition can comprise an aforementioned biodegradable polymer and one or more bioactive agents attached thereon or thereto. In one example, a bioactive composition can comprise a biodegradable polymer and multiple molecules of a same bioactive agent attached thereon or thereto (FIG. 8A-FIG. 8I). In another example, a bioactive composition can comprise a biodegradable polymer and multiple molecules of two or more bioactive agents attached thereon or thereto, such as one or more small molecule drugs and IgG or multiple different bioactive agents (FIG. 9A-FIG. 9B). A biodegradable polymer linked to an IgG, such as those shown in FIG. 9A and FIG. 9C, can be used to deliver the bioactive composition to a specific target or targets, for example tumor cells, via an antitumor IgG that binds to a tumor antigen as describe hereafter. A biodegradable polymer can also comprise a linker that links a biodegradable polymer and a bioactive agent. A linker can be a polymer, such as a polymeric linker, including, but not limited to, polyethyleneoxide (PEO), polyethylene glycol (PEG), poly (2-methyloxazoline), poly (2-ethyloxazoline), etc. Commercially available linkers, such as SMCC, MAL-PEG, MAL-PEG-NHS and others can be suitable. One or more bioactive agents or one or more biodegradable polymers can be linked with one or more linkers. Some examples are schematically shown in FIG. 9D-FIG. 9E.

A biodegradable polymer and a bioactive agent can be linked via a covalent bond or a non-covalent linkage. Any of covalent bonds, such as polymer bonds, linker mediated covalent bonds and other bonds can be suitable. Non-covalent linkage can include, but not limited to, such as, an electrostatic interaction, hydrophobic interaction, hydrophilic interaction, hydrogen bonding, physical interactions such as physical trapping or encapsulation, and so on.

A bioactive agent can also comprise a protein, a recombinant protein, an antibody, F_{ab}, antibody fragments, other antibody fragments that bind antigen, enzymes, a DNA, a recombinant DNA, DNA fragments, an RNA, an RNAi, an siRNA, a messenger RNA (mRNA), a recombinant RNA, RNA fragments, nucleotides, viruses, virus fragments or a combination thereof. A bioactive agent can be selected from a peptide, a monoclonal antibody, a fragment of a monoclonal antibody, a polyclonal antibody, a fragment of a polyclonal antibody, a synthetic antibody, a fragment of a synthetic antibody, or a combination thereof. A bioactive agent can comprise, for example, proteins, such as, enzymes, such as, L-asparaginase, antibodies and antigen-binding portions thereof, such as, alemtuzumab, bevacizumab, cetuximab, ibritumomab, rituximab, trastuzumab, gemtuzumab, checkpoint inhibiting antibodies including anti-PD1 antibodies (such as Keytruda or pembrolizumab, Opdivo or nivolumab, Bavencio or avelumab, Imfinzi or durvalumab, Tecentriq or atezolizumab), anti-PD-L1 antibodies, anti-CTLA-4 (cytotoxic T lymphocyte-associated antigen, also known as CD152) antibodies, anti-LAG3 (lymphocyte activation gene-3) antibodies, anti-TIM-3 (T cell immunoglobulin and mucin domain-3) antibodies, anti-CD19 antibodies, anti-CD20 antibodies such as tositumomab, cytokines, such as, interleukins, interferon α2a, interferon α, granulocyte colony stimulating factor (G-CSF) or Neupogen also known as Filgrastim, T-cell receptor (TCR), chimeric antigen receptor or chimeric antigen T-cell receptor (CAR-T), peptide hormones, such as, insulin, glucagon, glucagon like peptide-1, erythropoietin (EPO), thyroperoxidase (TPO), follicle stimulating hormone and so on, ligands of cell surface receptors, lectins, nucleic acids, such as siRNAs, ribozymes, antisense nucleic acids, naked nucleic acids and so on, viruses, virus-like particles and the like. Examples include Ecallantide.

Further examples of a bioactive agent can include recombinant blood factors, such as, factor III, antihemophilic factor, factor VIII, antithrombin, thrombin, factor VIIa, factor IX; tissue plasminogen activator, such as, TNK-tPA, tenecteplase and alteplase, including truncated forms thereof, such as, reteplase, hirudin, protein C and so on; recombinant hormones, such as, insulin, such as, insulin detemir, along-acting insulin analog, insulin glulisine, a rapid-acting insulin analog and insulin glargine (another long-acting insulin analog); human growth hormone, also known as somatropin, follicle-stimulating hormone, such as, the a subunit thereof, such as, corifollitropin a, glucagon like peptide-1, parathyroid hormone, and truncated forms thereof, such as, terpiparatide, B-type natriuretic peptide, calcitonin, luteinizing hormone, hCG, TSH, glucagon and so on; recombinant growth factors, such as, erythropoietin, such as, epoetin θ, erythropoietin α and epoetin β, long acting analogs thereof, such as, darbepoetin α; colony stimulating factors, such as, GM-CSF and G-CSF, insulin-like growth factor (IGF), a complex of IGF and IGF binding proteins, such as, mecasermin rinfabate, keratinocyte growth factor, platelet-derived growth factor and so on; recombinant cytokines, such as, interferons and interleukins, such as, interferon α, IFN-α-2b, interferon β, interferon-β-1B, IFN-β-1a, IL-11, IL-2, IFN-γ1b and so on; recombinant vaccines, such as those against hepatitis B, papillomavirus (HPV), cholera toxin B subunit, OspA (a lipoprotein found on the surface of B. burgdorferi), pertussis toxin and so on; monoclonal antibody and antigen-binding portions thereof, made to any antigenic entity as known in the art, such as, denosumab, tocilizurmab, besilesomab, ofatumumab, canakinumab, catumaxomab, golimumab, steknumab, ranibizumab, eculizumab, panitumumab, natalizumab, omalizumab, ibritumonmab, cetuximab, efalizumab, adalimumab, tositumomab, infliximab, palivizumab, daclizumab, votumumab, basiliximab, sulesomab, igovomab, abciximab and so on; other recombinant biologics, such as, bone morphogenetic proteins, such as, BMP-7 and BMP-2, and so on; recombinant enzymes, such as, α glucosidase, glucocerebrosidase, iduronate-2-sulfatase, N-acetylgalactosidase, 4-sulfatase, β-glucocerebrosidase, DNase, hyaluronidase, α-galactosidase, α-L-iduronidase, urate oxidase and so on; oligonucleopeptides; and so on, as well as combinations thereof, such as, rilonacept (a dimeric fusion protein of the extracellular (EC) domain of the IL-1 receptor and the F_{c} portion of an IL-1 IgG-1), romiplostim (a dimeric fusion protein with each monomer consisting of two thrombopoietin receptor-binding domains and the F_{c} region of an IgG-1), Abatacept (an immunoglobulin fused to the EC domain of CTLA-4), alefacept (containing the F_{c} portion of an antibody and a portion of CFA-3) and so on; anti-microbial or anti-virus antibody, such as such as anti-Ebola virus antibodies, fragments of an anti-Ebola virus antibodies, anti-B. anthracis antibodies (Anthrax antibodies), fragments of anti-B. anthracis antibodies; anti-Marburg virus antibodies, fragments of anti-Marburg virus antibodies, anti-Zika virus antibodies, fragments of anti-Zika virus antibodies, anti-Y. pestis antibodies, fragments of anti-Y. pestis antibodies, anti-F. tularensis antibodies, fragments of anti-F. tularensis antibodies, anti-Venezuelan equine encephalitis antibodies, fragments of anti-Venezuelan equine encephalitis antibodies, anti-brucella antibodies, fragments of anti-brucella antibodies, anti-smallpox antibodies, fragments of anti-smallpox antibodies, anti-botulinum toxin antibodies, fragments of anti-botulinum toxin antibodies, anti-ricin antibodies, fragments of anti-ricin antibodies, anti-V. cholerae antibodies, fragments of anti-V. cholerae antibodies, anti-C. burnetiid antibodies, fragments of anti-C. burnetiid antibodies, anti-salmonella antibodies, fragments of anti-salmonella antibodies, anti-listeria antibodies, fragments of anti-listeria antibodies, anti-E. coli antibodies, fragments of anti-E. coli antibodies; anti-inflammatory antibody such as anti-tumor necrosis factor (anti-TNF), anti-interleukin-1 (anti-IL-1) receptor, anti-IL-6 receptor, anti-α4 integrin subunit, and anti-CD20 agents; or a combination thereof. A fragment of an antibody can include an antigen-binding portion of the antibody. In a further example, the bioactive agent comprises one or more monoclonal antibodies, one or more antigen-binding portions thereof or a combination thereof, such as denosumab, tocilizurmab, besilesomab, ofatumumab, canakinumab, catumaxomab, golimumab, steknumab, ranibizumab, eculizumab, panitumumab, natalizumab, omalizumab, ibritumonmab, cetuximab, efalizumab, adalimumab, tositumomab, infliximab, palivizumab, daclizumab, votumumab, basiliximab, sulesomab, igovomab, abciximab, anti-PD1 antibodies, anti-PD-L1 antibodies, anti-CTLA-4 antibodies, anti-LAG3 antibodies, anti-TIM-3 antibodies, anti-CD19 antibodies, anti-CD20 antibodies, or a combination thereof.

Even further examples of a bioactive agent can include a variety of molecules, particularly those with the ability to bind to a target, such as another molecule, for example, a biological polymer, such as a polypeptide, a polynucleotide, a lipid, a polysaccharide, an enzyme, a receptor, an antibody, a vitamin, a lectin and so on. A target can be a pathogen, such as a parasite, a bacterium, a virus, or a toxin, such as a venom. A bioactive agent can be used for a variety of uses, including as a diagnostic agent, a therapeutic agent and so on. By “diagnostic agent” is meant a molecule which can be used as a marker for a particular disease, physiological state or stage, a pathological stage or state, and so on. Therapeutic agents are those that confer a beneficial effect in vivo, such as a drug, a nutrient, a protein and so on. It is not uncommon for a bioactive agent to be both a diagnostic agent and a therapeutic agent.

Further examples of a bioactive agent can also comprise cell-penetrating peptides (CPP) that can traverse the plasma membrane of cells and facilitate delivery of a bioactive agent to the cytoplasm or an organelle. Although the mechanism of CPP translocation is not clear, it is believed that CPP translocation can occur via direct penetration in the cell membrane, by endocytosis-mediated entry, or through the formation of a transitory structure that can translocate though a cell membrane. A bioactive composition comprising a biodegradable polymer, a cell-penetrating peptides (CPP) and an additional bioactive agent can be used.

Additional examples of a bioactive agent can comprise an agent for treatment of various cancers, such as a small molecule drug, a chemotherapy drug, biological or large molecule drugs including antibodies and monoclonal antibodies mentioned above and hereafter. A representative but non-limiting list of cancers include lymphoma, B cell lymphoma, T cell lymphoma, mycosis fungoides, Hodgkin's Disease, myeloid leukemia, bladder cancer, brain cancer, nervous system cancer, head and neck cancer, squamous cell carcinoma of head and neck, kidney cancer, lung cancers such as small cell lung cancer and non-small cell lung cancer, neuroblastoma/glioblastoma, ovarian cancer, pancreatic cancer, prostate cancer, skin cancer, liver cancer, melanoma, squamous cell carcinomas of the mouth, throat, larynx, and lung, colon cancer, cervical cancer, cervical carcinoma, breast cancer, epithelial cancer, renal cancer, genitourinary cancer, pulmonary cancer, esophageal carcinoma, head and neck carcinoma, large bowel cancer, hematopoietic cancers; testicular cancer; colon and rectal cancers, prostatic cancer, and pancreatic cancer.

In one example, a bioactive agent (BA) can be reacted with Traut's reagent to react with available amine groups followed by purification to produce BA-SH. A biodegradable polymer can react with MAL-PEG-NHS (NHS is N-hydroxysuccinimide, which reacts with an available amine group) and purified to produce biodegradable polymer-MAL. The biodegradable polymer-MAL then reacts with BA-SH to generate the final conjugate. In another example, biodegradable polymer reacts with Traut's reagent to produce polymer with reactive sulfhydryl groups, which then is purified, followed by reaction with BA-MAL (which derivatized oligonucleotide is made by reacting BA with SMCC as discussed herein and as known in the art) to generate the final conjugate.

Each BA molecule can be conjugated with one or more biodegradable polymers using such linking chemistry as known in the art. In one example, a BA is linked to two polymers via a multi-valent linker (FIG. 9E). In one example, the bivalent linker disclosed above can be modified with multi-amino, multi-imino, carboxyl, —SH, or —OH functional groups to produce a multi-valent linker, which allows the attachment of multiple polymers per BA. On the other hand, multiple BA molecules can be attached to one molecule of a biodegradable polymer, for example, via multiple functional groups, such as activate amine groups.

In a further example, the BA-MAL (made by reacting with SMCC) can react with a 4-Arm PEG Thio (which is available commercially) to attach the oligonucleotide to one, two or three of the four thio groups presented in the four arm structure, followed by reaction with maleimide functionalized biodegradable polymer (which can be made by reacting polymer with SMCC for reaction at available amine groups to produce the maleimide functions) to form a biodegradable polymer attached to one, two or three BAs. By altering the number of arms reacted with the PEG linker, two or three polymers can be attached thereto. In yet another example, the BA-NH₂ can react with a 4-Arm PEG polymer functionalized with epoxide or activated ester groups (which is commercially available), followed by reaction with amine functionalized biodegradable polymer. In yet further an example, the BA-SH can react with a 4-Arm PEG maleimide (commercially available), followed by reaction with thio functionalized biodegradable polymer (made, for example, by reacting polymer with iminothiolane). Schematic examples are shown in FIG. 9E. The reactions can be performed to minimize or eliminate crosslinked products, for example, by using known protection and deprotection reaction chemistries and schemes, and methods, by using a large excess of reagents, and so on.

The term “antibody” or “antibodies” can include natural or synthetic antibodies that selectively bind to an antigen. The term includes polyclonal and monoclonal antibodies. In addition to intact immunoglobulin molecules, also included in the term “antibodies” are fragments or polymers of those immunoglobulin molecules, and human or humanized versions of immunoglobulin molecules that selectively bind a target antigen.

Antibodies that can be used in the disclosed compositions and methods include whole immunoglobulin (i.e., an intact antibody) of any class, fragments thereof, and synthetic proteins containing at least the antigen binding variable domain of an antibody. The variable domains differ in sequence among antibodies and are used in the binding and specificity of each particular antibody for cognate antigen (as referred to as a binding pair). However, variability is not usually evenly distributed through the variable domains of antibodies. It is typically concentrated in three segments called complementarity determining regions (CDRs) or hypervariable regions both in the light chain and the heavy chain variable domains. The more highly conserved portions of the variable domains are called the framework (FR). The variable domains of native heavy and light chains each comprise four FR regions, largely adopting a β-sheet configuration, connected by three CDRs, which form loops connecting, and in some cases forming part of, the β-sheet structure. The CDRs in each chain are held together in close proximity by the FR regions and, with the CDRs from the other chain, contribute to formation of an antigen binding site of an antibody.

Also disclosed are fragments of antibodies which have bioactivity. Fragments, whether attached to other sequences or not, include insertions, deletions, substitutions, or other selected modifications of particular regions or specific amino acids residues, provided the activity of the fragment is not significantly altered or impaired compared to the nonmodified antibody or antibody fragment. Fragments of antibodies can be suitable.

The term “antibody” or “antibodies” also includes single-chain antibodies specific to an antigen. Methods for production of single-chain antibodies are known to those of skill in the art. A single chain antibody can be created by fusing variable domains of heavy and light chains using a short peptide linker, thereby reconstituting an antigen binding site on a single molecule. Single-chain antibody variable fragments (scFᵥs) in which the C-terminus of one variable domain is tethered to the N-terminus of the other variable domain via a 15 to 25 amino acid peptide or linker have been developed without significantly disrupting antigen binding or specificity of binding. A linker is chosen to permit a heavy chain and a light chain to bind in proper conformational orientation.

Divalent single-chain variable fragments (di-scFᵥs) can also be suitable and can be engineered by linking two scFᵥs. That can be done, for example, by producing a single peptide chain with two V_{H} and two V_{L} regions, yielding tandem scFᵥs. scFᵥs can also be designed with linker peptides that are too short for the two variable regions to fold together (about five amino acids), forcing scFᵥs to dimerize. That construct is known as a diabody. Diabodies have been shown to have dissociation constants up to 40-fold lower than corresponding scFᵥs, meaning a much higher affinity to a target, i.e., a member of a binding pair. Still shorter linkers (one or two amino acids) lead to formation of trimers (triabodies or tribodies). Tetrabodies have also been produced, which exhibit higher affinity to targets as compared to diabodies of the same specificity. Diabodies, triabodies and tetrabodies can also be suitable.

In one embodiment, an oligodeoxynucleotide can be attached to a biodegradable polymer through a covalent bond, a non-covalent linkage, or a combination thereof. In another embodiment, an oligodeoxynucleotide can be attached to a biodegradable polymer through a linker having a covalent bond. In an embodiment, an oligodeoxynucleotide can be attached to a biodegradable polymer in a nanoparticle via non-covalent linkage, such as, an electrostatic interaction, hydrophobic interaction, hydrophilic interaction, hydrogen bonding and so on. A bioactive agent can be contained within a nanocapsule/nanoparticle or can be attached to a nanocapsule/nanoparticle. In another embodiment, an antibody can be attached to a biodegradable polymer through a covalent bond, a non-covalent linkage, or a combination thereof. In yet another embodiment, an antibody can be attached to a biodegradable polymer through a linker, for example, using a carboxyl, amino or thio group, for example, of an antibody and conjugating a linker thereto practicing known materials and methods, for example, as described in Bioconjugation Techniques (G. T. Hermanson, Elsevier, 2013). In an embodiment, an antibody can be attached to a biodegradable polymer in a nanoparticle via any of the aforementioned non-covalent linkage.

In any bioactive compositions of this invention, a bioactive agent can comprise a vaccine. A vaccine can comprise an antigen, a toxin, a modified or disabled toxin including natural or synthetic molecules that can cause immunoreaction in a biosystem such as in humans or animals. A vaccine can be attached to a biodegradable polymer via one or more covalent bonds, non-covalent linkages, or a combination thereof. Commercial vaccines and the vaccines listed by US Centers for Disease Control and Prevention (CDC) can be suitable.

A bioactive agent can comprise a DNA or an mRNA vaccine encoding at least one antigen, or a combination of a DNA and an mRNA vaccine thereof. A DNA or an mRNA vaccine can include conventional GC-rich nucleotides with protamine or a lipid formulation, self-amplifying mRNA, in vitro synthesized nucleoside mRNA or DNA that encodes a single antigen, a plurality of antigens, neoantigens that are based on nucleic acid mutations and/or polymorphism such as an SNP (single nucleotide polymorphism), or a combination thereof.

In one example, a vaccine comprises a plasmid that comprises DNA sequences encoding at least one antigen, epitope or determinant that can be expressed in a cell under the control of a suitable promoter to produce mRNA, protein, or a combination thereof. In another example, a vaccine comprises mRNA encoding a plurality of antigens, epitopes or determinants, such as MHC (major histocompatibility complex) epitopes that can be used for treating cancer or other diseases. MHC Class II neo-epitopes based on confirmed mutations can be suitable. An mRNA vaccine can comprise multiple neo-antigen sequences presented in an mRNA. In yet another example, an mRNA vaccine comprises a sequence having a 5′-untranslated region (5-′UTR), an mRNA sequence encoding at least one antigen or a neoantigen and a 3′-untranslated region (3′-UTR) assembled as a transcription cassette or unit. In a further example, a vaccine can comprise an mRNA encoding one or more antigens or epitopes specific to Ebola virus, Zika virus, Anthrax, or a combination thereof. In yet another example, a vaccine comprises DNA or mRNA encoding one or more cancer specific antigens, epitopes or a combination thereof.

A DNA or an mRNA vaccine can be attached to a biodegradable polymer of this invention via one or more covalent bonds or non-covalent linkages. In one example, a DNA or an mRNA is attached to a biodegradable polymer via one or more covalent bonds, such as an amide linkage, an ester linkage, an ether linkage, an —S—S— bond, an —N—C— bond, an —S—C bond, an —O—C bond, or a combination thereof practicing materials and methods described herein or as known in the conjugation art. In another example, a DNA or an mRNA vaccine is mixed together with a biodegradable polymer to form a bioactive composition via non-covalent linkages.

A bioactive agent described herein can include a chemical compound or a small molecule drug, a chemotherapy drug, an inorganic-based drug, a biological or large molecule drug, modifications and/or derivatives thereof, and combinations thereof.

Chemical compounds or small molecule drugs can include any water soluble, substantially poorly water soluble or water insoluble pharmacologically active agents. Some of those may need to be converted to a less water soluble form, for example, changing the pharmaceutically active agent from a salt to a non-salt form or from a charged to a non-charged molecule. Others may need to be converted to a more water soluble form, for example, converting a compounds to a salt or to comprise hydrophilic functional groups. Biodegradable polymers having hydrophobic domains, such as those having hydrocarbon chains, for example C3-C30 hydrocarbon chains, can have advantages for forming conjugates with water insoluble or substantially poorly water soluble bioactive agents disclosed herein. Bioactive agents having negative charges can also have suitable interactions with biodegradable polymers of this invention that comprises cationic components.

Further examples of a bioactive agent can include growth agents, AIDS adjunct agents, alcohol abuse preparations, such as, agents for treating dependence or withdrawal, Alzheimer's treatments, amyotrophic lateral sclerosis treatments, analgesics, anesthetics, anticonvulsants, antidiabetic agents, antidotes, antifibrosis therapies, antihistamines, anti-infective agents, such as, antibiotics, antivirals, antifungals, amebicides, antihelmintics, antimalarials, leprostatics and so on, antineoplastics, antiparkinsonian agents, antirheumatic agents, appetite stimulants, biological response modifiers, biologicals, blood modifiers, such as, anticoagulants, colony stimulating factors, hemostatics, plasma extenders, thrombin inhibitors and so on, bone metabolism regulators, cardioprotective agents, cardiovascular agents, such as, adrenergic blockers, adrenergic stimulators, angiotensin converting enzyme (ACE) inhibitors, antiarrhythmics, antilipemic agents, calcium channel blockers, diuretics, vasopressors and so on, central nervous system (CNS) stimulants, cholinesterase inhibitors, contraceptives, fertility treatments, ovulation stimulators, cystic fibrosis managements agents, detoxifying agents, diagnostics, dietary supplements, dopamine receptor agonists, endometriosis management agents, enzymes, erectile dysfunction treatments, foot care products, gastrointestinal (GI) agents, such as antacids, antidiarrheals, antiemetics, antiflatulants, bowel evacuants, digestive enzymes, histamine receptor agonists, laxatives, proton pump inhibitors, prostaglandins and so on, Gaucher's disease treatments, gout treatments, homeopathic remedies, skin treatments, vitamins, nutrients, hormones, hypercalcemia management treatments, hypocalcemia management treatments, immunomodulators, immunosuppressants such as rapamycin, levocarnitine deficiency treatments, mast cell stabilizers, migraine treatments, motion sickness products, such as, benadryl and phenergan, decongestants, antihistamines, cough suppressants, multiple sclerosis treatments, muscle relaxants, nasal preparations, such as, antiinflammatories, smoking cessation aids, appetite suppressants, nucleoside analogs, obesity managements, ophthalmic preparations, such as, antibiotics, antiglaucoma agents, artificial tears, lubricants and so on, sexual aids, lubricants, osteoporosis treatments, otic preparations, such as, antiinfectives and cerumenolytics, minerals, oxytocics, parasympatholytics, parasympathomimetics, patent ductus arteriosus agents, phosphate binders, porphyria agents, prostaglandins, psychotherapeutic agents, radiopaque agents, respiratory agents, such as, antiinflammatories, antitussives, bronchodilators, decongestants, expectorants, leukotrienes antagonists, surfactants and so on, salt substitutes, sedatives, hypnotics, skin and mucous membrane preparations, such as, acne treatments, anorectal treatments, such as, hemorrhoid treatments and enemas, antiperspirants, antipruritics, antipsoriatic agents, antiseborrheic agents, burn treatments, cleansing agents, depigmenting agents, emollients, hair growth retardants, hair growth stimulators, keratolytics, hair problem treatments, mouth and throat problem treatments, shampoos, photosensitizing agents, wart treatments, wound care treatments and so on, over the counter pharmaceutics and products, such as, deodorants, Tourette's syndrome agents, tremor treatments, urinary tract agents, such as, acidifiers, alkalinizers, antispasmodics, benign prostatic hyperplasia treatments, calcium oxalate stone preventors, enuresis management agents and so on, vaginal preparations, such as, antiinfectives, hormones and so on, vasodilators, vertigo treatments, Wilson's disease treatments and so on.

In some examples, in a bioactive composition of this invention, a bioactive agent can comprise a DNA or an mRNA vaccine encoding at least one antigen, or a combination of the DNA and the mRNA vaccines thereof.

A bioactive composition of this invention can further comprise a second polymer selected from a linear polymer, a branched polymer, a block copolymer, a graft copolymer, a dendrimer, or a combination thereof. The second polymer can comprise a cationic polymer. A cationic polymer comprising at least one end amine group selected from —N(CH₂)NH₂, —N(CH₂)₂NH₂, —N(CH₂)₃NH₂, —N(CH₂)N⁺H₃, —N(CH₂)₂N⁺H₃, —N(CH₂)₃N⁺H₃, or a combination thereof, polymerized ethyleneimine (PEI), polymerized propyleneimine (PPI), polymerized amidoamine (PAMAM), tris(2-aminoethyl)amine (TREN), polymerized tris(2-aminoethyl)amine, polyalkylamine, polyallylamine, or a combination thereof, can be suitable. For a bioactive composition of this invention, the biodegradable polymer and the second polymer can be the same or different. A bioactive composition of this invention can comprise a biodegradable polymer of this invention and a second polymer that is a non-biodegradable polymer. In one example, a bioactive composition can comprise a biodegradable polymer having a polylysine core modified with one or more ethyleneimine (EI) or polyethyleneimine (PEI) and a second polymer comprising polymerized amidoamine (PAMAM). In another example, a bioactive composition can comprise a biodegradable dendritic polymer having a PEI12 core, two or more layers of polylysine and further modified with one or more ethyleneimine (EI) or polyethyleneimine (PEI), such as Den(PEI12-PLK2-EI) or Den(PEI12-PLK3-EI) described in Examples, and a second polymer comprising a polyethyleneimine (PEI) dendrimer. In yet another example, a bioactive composition can comprise a combination of two or more different biodegradable polymers of this invention. In a further example, a bioactive composition can comprise one of biodegradable polymers of this invention and a second polymer comprising a polyoxazoline (PEOX).

The second polymer can have a molecular weight in a range of from 200 to 100 KDa. The second polymer can have a molecular weight in a range of from 200 to about 100 KDa. The PEI can have molecular weight in a range of from of about 200 to 800 KDa in one example, 1.0 to 2.0 KDa in another example, 2.0 to 5.0 KDa in yet another example, 5.0 to 10 KDa in yet another example, 10 to 20 KDa in yet another example, 20 to 25 KDa in yet another, 25 to 100 KDa in a further example, or a combination thereof.

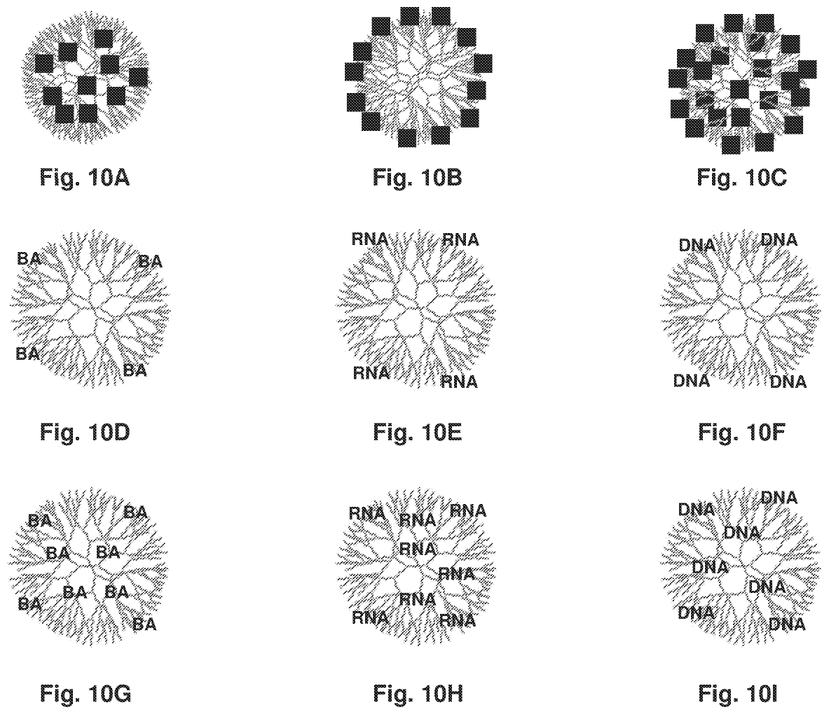

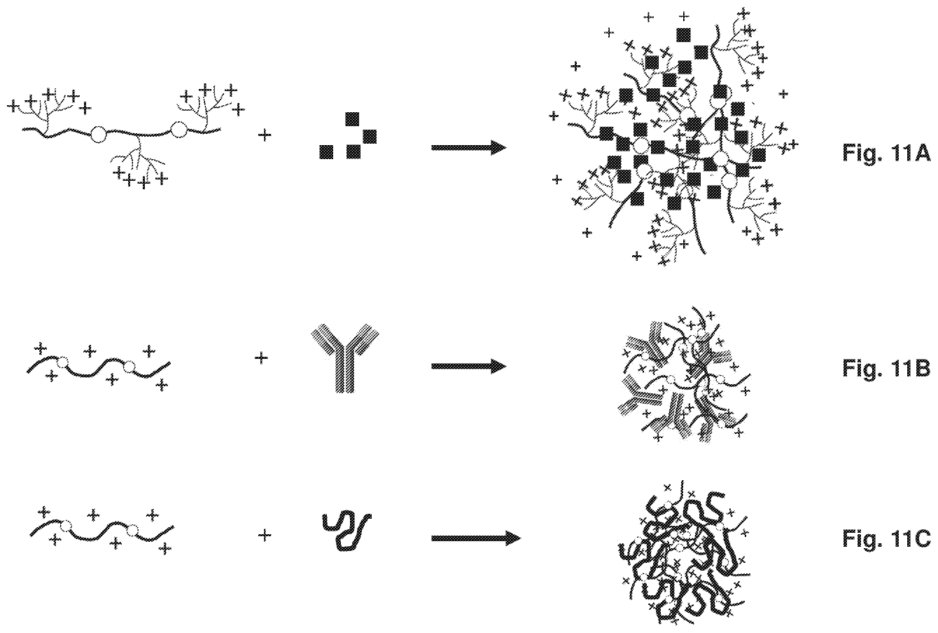

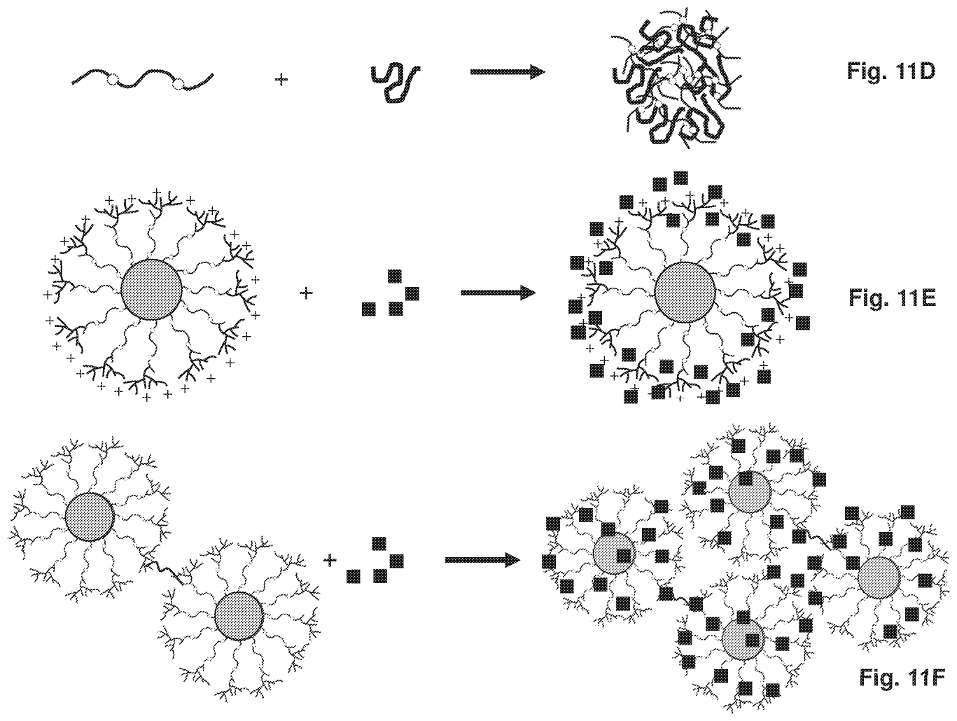

A bioactive composition of this invention can comprise at least two bioactive agents. The two bioactive agents can be the same or different. In one example, a bioactive composition of this invention can comprise two or more molecules of a same bioactive agent covalently, non-covalently, or a combination thereof, such as schematically illustrated in FIG. 6, FIG. 7B, FIGS. 8A-I, FIGS. 10A-F and FIGS. 11A-F. In another example, a bioactive composition of this invention can comprise two or more molecules of different bioactive agents covalently, non-covalently, or a combination thereof, such as schematically illustrated in FIG. 9A-FIG. 9B. A combination of two or more different biodegradable polymers and bioactive agents can also be suitable. A first of the at least two bioactive agents can comprise a first RNA, mRNA, RNAi, siRNA, microRNA, oligonucleotide, DNA, oligodeoxynucleotide, chemical compound, chemotherapy drug, small molecule drug, or a combination thereof and a subsequent of the at least two bioactive agents can comprise a second RNA, mRNA, RNAi, siRNA, microRNA, oligonucleotide, DNA, oligodeoxynucleotide, chemical compound, chemotherapy drug, small molecule drug, protein, peptide, antibody, monoclonal antibody (mAb), fragment of an antibody, or a combination thereof. In one example, a first bioactive agent can comprise a chemotherapy drug and a second bioactive agent can comprise a second chemotherapy drug, monoclonal antibody (mAb) including one or more aforementioned checkpoint inhibiting antibodies, or a combination thereof. In a further example, a bioactive composition can comprise any biodegradable polymers disclosed herein and at least two bioactive agents selected from a taxane (or paclitaxel), gemcitabine, one or more checkpoint inhibiting antibodies, or a combination thereof. In yet a further example, a bioactive composition can comprise any biodegradable polymers disclosed herein and at least two bioactive agents selected from an oligodeoxynucleotide (ODN), a taxane (or paclitaxel), gemcitabine, one or more checkpoint inhibiting antibodies, or a combination thereof.

A bioactive composition of this invention can further comprise a second oligodeoxynucleotide (ODN) that is attached to a biodegradable polymer through a covalent linkage, attached to a second polymer (when present) through a covalent linkage, or a combination thereof. The second ODN can be the same or different from the first ODN when present. The second ODN can be covalently linked via amine groups, phosphate groups, or a combination thereof. In one example, one or more NH₂ groups of ODN can be reacted with a Traut's reagent followed by purification to produce ODN-SH. A second polymer (either biodegradable or non-biodegradable polymer), for example, a branched PEI polymer or a PAMAM dendrimer, can react with a hetero functional linker such as Maleimide (MAL)-PEG-NHS ester and purified to produce polymer-MAL. The polymer-MAL can then react with ODN-SH to generate the final polymer-ODN conjugate. In another example, a second polymer can react with a Traut's reagent, purified, followed by a reaction with ODN-SMCC to generate a final polymer ODN conjugate. Each of the ODN molecules can be conjugated with one or more polymers. In one example, one ODN can be linked to two polymers via a multi-valent linker. In another example, the bivalent linker disclosed above can be modified with multi-amino, multi-imino, carboxyl, or —SH, or —OH functional groups to produce a multi-valent linker, which allows the attachment of multiple polymers per ODN (shown as bioactive agent BA) (FIG. 9E). As disclosed herein, a second polymer can be a biodegradable or a non-biodegradable polymer. In an example, a bioactive composition comprises a biodegradable polymer of this invention and a non-covalently associated first ODN, a second polymer and a second ODN that is attached to the biodegradable polymer through a covalent linkage, attached to the second polymer through a covalent linkage, or a combination thereof. In another example, a bioactive composition comprises a biodegradable polymer of this invention and a non-covalently associated first ODN, a second polymer and a non-covalently associated second ODN. In any of the examples, the first ODN and the second ODN can be the same or different.

For a bioactive composition of disclosed herein, a bioactive agent can further comprise an inhibitor or an activator of a bioprocess. In one example, a bioactive agent can comprise an ODN as a first bioactive agent and an inhibitor or a drug, such as an aforementioned small molecule drug, anti-PD1 antibodies, anti-PD-L1 antibodies, anti-CTLA-4 antibodies, anti-LAG3 antibodies, anti-TIM-3 antibodies, anti-CD19 antibodies, anti-CD20 antibodies, or a combination thereof, as an inhibitor or an activator of a bioprocess for a treatment, such as, a cancer treatment.

A bioactive composition comprising a biodegradable polymer and at least one bioactive agent disclosed herein can form nanoparticles having particle sizes in a range of from 1 nm to 1000 nm. The nanoparticles can be soluble or dispersible in aqueous solutions. The biodegradable polymer and the bioactive agent can form nanoparticles by covalent bonds as described above or by non-covalent linkages, such as electrostatic interaction, hydrophobic interaction, hydrophilic interaction, metal chelation, hydrogen bonding, physical trapping or encapsulation, or a combination thereof. Nanoparticles can be soluble or dispersible in aqueous solutions. Nanoparticles can have a size in a range of from 1 nm to 1,000 nm in an example, 5 nm to 1000 nm in another example, 10 nm to 1000 nm in yet another example, 20 nm to 1000 nm in yet another example, 30 nm to 1000 nm in yet another example, and 50 nm to 1000 nm in yet another example. Nanoparticles can also have a size in a range of from 1 nm to 900 nm in an example, 1 nm to 700 nm in another example, 1 nm to 500 nm in yet another example, 1 nm to 300 nm in yet another example, 1 nm to 200 nm in yet another example, 1 nm to 100 nm in yet another example, 1 nm to 50 nm in yet another example, and 1 nm to 10 nm in yet another example. Nanoparticles can also have a size in a range of from 1.5 nm to 50 nm in a further example. The size of nanoparticles can be measured as a mean diameter of the nanoparticles.

An aqueous solution can comprise in a range of from 80% to 100% of water, percent based on total weight of the aqueous solution. In one example, an aqueous solution can comprise 80% to 100% of water and 0 to 20% of one or more organic solvents. Organic solvents can be water miscible or non-miscible. In one example, an organic solvent comprises a water miscible organic solvent. Nanoparticles can also be dispersed in an organic solvent and then that solution is converted to an aqueous solution via a suitable process, such as, emulsification.

Some representative examples of nanoparticles comprising a biodegradable polymer are schematically shown in FIG. 10A-FIG. 10I. A bioactive agent can be inside nanoparticles, at the surface of nanoparticles, or spread throughout nanoparticles. In one example, a ODN (shown as a bioactive agent BA) is attached to the surface of nanoparticles, for example, by one or more covalent bonds or by non-covalent linkages, such as electrostatic interaction, hydrophobic interaction, hydrophilic interaction, metal chelation, hydrogen bonding or a combination thereof. In another example, DNA or RNA is attached to the surface of nanoparticles, for example, by one or more covalent bonds or by non-covalent linkages, such as electrostatic interaction, hydrophobic interaction, hydrophilic interaction, metal chelation, hydrogen bonding or a combination thereof. FIG. 10G-FIG. 10I show examples of ODN (BA), DNA and RNA including mRNA that are non-covalently encapsulated with biodegradable polymer nanoparticles.

Nanoparticles can be formed in solution by any of the methods taught herein. Nanoparticles can be lyophilized for stability and long-term storage. Lyophilized nanoparticles comprising a biodegradable polymer and a bioactive agent can be reconstituted with an aqueous solution or an organic solvent. In one example, lyophilized nanoparticles are reconstituted in an aqueous solution. In another example, reconstituted nanoparticles are used for administrating into a subject for treating a disease.

Nanoparticles comprising biodegradable polymers and an aforementioned bioactive agent can also be produced by associating a biodegradable polymer and a bioactive agent in an aqueous solution, an organic solvent, or a combination thereof. That can be suitable for producing nanoparticles from biodegradable polymers and bioactive agents that are linked via non-covalent linkages. A biodegradable polymer and a bioactive agent can be dissolved in aqueous solutions separately and then mixed together forming desired nanoparticles. A biodegradable polymer and a bioactive agent can also be dissolved together forming desired nanoparticles. Nanoparticles can be frozen at −80° C. and then lyophilized to produce dried nanoparticles or aggregates.

For a bioactive agent that is not water soluble, such as taxane or paclitaxel, the bioactive agent can be first dissolved in an organic solvent, such as ethanol, methanol, acetone, or a mixture thereof. A biodegradable polymer can be dissolved in an aqueous solution, such as water, saline or a buffer. A biodegradable polymer can also be dissolved in water miscible or water non-miscible organic solvent. A water miscible organic solvent can comprise or be selected from methanol, ethanol, acetone, acetic acid, or the like, or a combination thereof. A bioactive agent solution and a biodegradable polymer solution can be mixed to form nanoparticles. The mixture can be frozen at −80° C. and then lyophilized to produce dried nanoparticles or aggregates.

Some representative examples of nanoparticles are schematically shown in FIG. 11A-FIG. 11F that can have biodegradable polymer associated with one or more bioactive agents: FIG. 11A, linear polymer and small molecule bioactive agent; FIG. 11B, linear polymer and an antibody or a purified IgG or a fragment thereof, similarly an antigen or an antigen fragment thereof can also be associated with a biodegradable polymer; FIG. 11C, linear polymer and large molecule bioactive agent, such as a protein or peptide; FIG. 11D, biodegradable polymer having different electric charge under different environment, such as at different pH conditions; FIG. 11E, dendrimer and small molecule bioactive agent; and FIG. 11F, covalently linked polymers and small molecule bioactive agent. Charges can vary before, during or after the formation of the nanoparticles. Typically, a biodegradable polymer can exhibit positive charge in vivo where pH is low. Charges of a biodegradable polymer can be adjusted by using acid, base, buffer, or a combination thereof. In one example, a biodegradable polymer can have positive charge under acidic or neutral pH conditions to affiliate with a negatively charged bioactive agent, such as a nucleic acid, an RNA, a DNA, an oligodeoxynucleotide and the like to produce a bioactive composition. In another example, a biodegradable polymer can have neutral or negative charge under basic pH conditions to affiliate with a positively charged bioactive agent to produce a bioactive composition. Charge variation of a biodegradable polymer may help to dissociate a bioactive agent in vivo once a bioactive agent is delivered to a targeted in vivo location. A biodegradable polymer and a bioactive agent can be linked via non-covalent linkages.

In one example, an ODN is dissolved in an aqueous solution, such as, water, a buffer, a saline and so on, which can further comprise an isotonic agent, a carbohydrate, such as, glucose, and so on, and mixed with a biodegradable polymer of this invention that is also dissolved in the same aqueous solution to produce an ODN-biodegradable polymer mixture, which may be simply mixed or formed into nanoparticles through, for example, electrostatic interaction, wherein the ODN and the biodegradable polymer are linked non-covalently. Such a mixture or nanoparticles can be used as a generic immune system stimulator or enhancer, such as, but not limited to, an adjuvant, and can be used with a second bioactive agent as taught herein, for example, in a combination treatment, such as, with a vaccine, a cancer therapy drug including small molecule drugs, chemotherapy drugs, monoclonal antibodies, and so on. A biodegradable polymer of this invention can be used to enhance immunoreaction, reduce drug dosage needed for treating a disease, reduce toxic side effects, or a combination thereof. In one example, a biodegradable polymer disclosed herein, can be mixed with a bioactive agent, such as an mRNA vaccine, an ODN, or a combination thereof, and can be administered to a subject. One or more second bioactive agents, such as a drug, for example, any of the bioactive agents, chemotherapy drugs, or a combination thereof, can be suitable. In another example, a second bioactive agent can be Paclitaxel, Gemcitabine, Cisplatin, or a combination thereof.

In another example of a bioactive composition, a biodegradable polymer and one or more bioactive agents can be simply mixed together, co-lyophilizing, co-dry, or a combination thereof, and can be used as described above.

Any bioactive compositions disclosed above can also comprise a subsequent polymer selected from a linear polymer, a branched polymer, a block copolymer, a graft copolymer, a dendrimer, or a combination thereof. Typical biopolymers, such as polysaccharides, polycarbohydrates, proteins, polypeptides, polynucleoacids, lipids, or a combination thereof; non-biopolymers, such as acrylic polymers, polyesters, polyurethanes, latex, silicane, silicone, or a combination thereof, can be suitable. The second or the subsequent polymer can also be used to modify, such as, to add, a property of a bioactive composition and/or a nanoparticle, for example, viscosity, stability, solubility, particle size, and so on.

Any aforementioned bioactive composition can further comprise a carrier. Pharmaceutically acceptable carriers including pharmaceutical excipient and inactive ingredients can be suitable. A carrier can be a pharmaceutically acceptable carrier that can be administered to a subject to provide an effective dose of an active ingredient and in compliance with government regulations. A carrier can be selected from a detergent, a buffer, a phosphate, a salt, water, a solvent, a filler, an inorganic compound, an organic compound, a synthetic polymer, a biopolymer, or a combination thereof. A carrier can comprise those listed in in current and updated US FDA (Food and Drug Administration) inactive ingredient database (IID), reagents determined to be generally regarded as safe (GRAS), or a combination thereof.

A bioactive composition of this invention can be a pharmaceutical composition for treating a disease of a subject in need thereof. The term “disease”, “diseases” or a grammatical variation refers to a cancer, a tumor, a neoplastic disorder, an infectious disease, a metabolic disease, any impairment of the normal condition of a human being that leads to abnormal functions, or a combination thereof. In one example, the bioactive composition is a pharmaceutical composition for treating a cancer, an infectious disease, an autoimmune disease, or a combination thereof.

A bioactive composition of this invention can further comprise a targeting agent for targeting the bioactive composition to at least one target. The targeting agent can comprise a physical targeting agent, a chemical targeting agent, a biological targeting agent, or a combination thereof. Some examples of targeting agent can include, but not limited to, magnetic targeting agent that can be guided to a specific location in a biosystem via magnetic field, a pH sensitive targeting agent that can acuminate or be delivered to a location having a specific pH range such as acidic or basic pH range, an antigen/antibody based targeting agent, a ligand/receptor based targeting agent, or any other biological binding pair-based targeting agents. Examples of binding pairs can include an antibody or an antigen-binding portion thereof, an antigen; an avidin/streptavidin/neutravidin, a biotin; a dinitrophenol (DNP), an anti-DNP antibody; a digoxin, an anti-digoxin antibody; a digoxigenin, an anti-digoxigenin antibody; a hapten, an anti-hapten antibody; a polysaccharide, a polysaccharide binding moiety, such as a lectin; a receptor, a ligand; a fluorescein, an anti-fluorescein antibody; a pair of complementary DNA; a pair of complementary RNA and anti-sense RNA; a pair of complementary DNA and RNA; etc. A targeting agent can be a member of a binding pair that can target the other member of the binding pair in a biosystem.

For a bioactive composition of this invention, a target can comprise a biosystem selected from cells in vitro, cells in vivo, nuclei of cells, cytoplasm of cells, extracellular matrix, tissues, body fluid of a subject, blood of the subject, one or more organs of the subject, one or more tumors of the subject, or a combination thereof. The term “organs”, “organ” or a grammatical variation refers to body fluid, blood, lymphoid fluid or lymphoid organ, bone marrow, GI tracts, bile, pancreas, liver, lung, heart, or any other solid or fluid parts of a biosystem, such as a human patient. The target can also comprise one or more cells selected from T cells, B cells, NK (natural killer) cells, cancer cells, tumor cells, antigen presenting cells (APC), dendritic cells (DC), neutrophils, macrophages, lymphocytes, monocytes or a combination thereof. For example, an antibody against one or more cardiomyocyte markers can be used as a targeting agent for targeting a bioactive composition to heart or heart cells. In another example, an antibody or a binding protein that binds to alanine aminotransferase (ALT) biomarker can be used for targeting liver or one or more ALT rich tissues or organs. In yet another example, a bioactive composition can comprise a binding member of T-cell antigen for targeting the bioactive composition to T cells. In yet another example, a bioactive composition can comprise an antibody against a tumor antigen for targeting the bioactive composition to tumor cells. In yet another example, a bioactive composition can comprise a bacteria binding member for targeting the bioactive composition to bacteria infected cells or tissues in a human patient or an animal. In yet another example, a bioactive composition can comprise an anti PD-1 antibody for targeting T cells in a patient. In yet another example, a bioactive composition can comprise an antibody against CD28, an antibody against TCR (T cell receptor), or a combination, for targeting T cells. In yet another example, a bioactive composition can comprise an antibody against CD38 for targeting B lymphocytes. In yet another example, a bioactive composition can comprise an antibody against human epidermal growth factor receptor type 2 (Her2/neu) for targeting breast cancer cells. In yet another example, a targeting agent can target Innate (NK) & Adaptive Immune System to trigger or to enhance immune responses of a subject, such as a patient. A combination of two or more targeting agents can also be suitable.

A biodegradable polymer of this invention can have a desired cytotoxicity that inhibits cell growth, kills cells or otherwise damages, destroys or lyses cells or tumors. Such biodegradable polymer with cytotoxicity can be linked to a targeting agent via one or more covalent bonds or non-covalent linkages to deliver the cytotoxicity to a specific target, such as tumor cells. In one example, a biodegradable polymer can comprise a PAMAM core, such as a PAMAM dendrimer having 5 layers PAMAM layer (E5), two or more layers of lysine residues and one or more hydrocarbon chains and an antibody against a tumor antigen on the surface of tumor cells as a targeting agent. Cytotoxicity can generally be modulated by adjusting the sizes (molecular weight), branching, functional groups, cationic components, hydrocarbon chain, of a biodegradable polymer, or a combination thereof.

One advantage of the biodegradable polymers of this invention is that any excess biodegradable polymers with cytotoxicity can be subsequently degraded in a biosystem, such as in a patient, resulting in reduced non-specific toxicity. As disclosed above and hereafter, biodegradable polymers of this invention can comprise multiple cationic components each is less than 2,000 Dalton so once a biodegradable polymer with cytotoxicity is degraded in a biosystem, these polymer breakdown products can exhibit no or low toxicity or exit the biosystem rapidly.

This invention is further directed to a method for delivering a bioactive agent into a biosystem. The method comprises the steps of:
associating any one or more of the aforementioned biodegradable polymers and a bioactive agent to produce a bioactive composition; and
introducing the bioactive composition to the biosystem.

In the method, the biosystem can be selected from cells in vitro, cells in vivo, nuclei of cells, cytoplasm of cells, extracellular matrix, tissues, body fluid of a subject, blood of the subject, an organ of the subject, a tumor of the subject, or a combination thereof.

Any of aforementioned biodegradable polymers of this invention can be suitable. The aforementioned biodegradable polymers and the bioactive agent can be associated via one or more covalent bonds or non-covalent linkages.

This invention is further directed to a method for treating a disease of a subject. The method comprises the steps of:
associating any of the biodegradable polymers and at least one bioactive agent disclosed herein to produce a bioactive composition; and
introducing the bioactive composition to the subject.

The biodegradable polymers and the at least one bioactive agent can be associated via covalent bonds or non-covalent linkages.

The method can further comprise the step of: subsequently, introducing a second bioactive agent to the subject after the bioactive composition is introduced. Any of the aforementioned bioactive agents, such as a chemotherapy drug or a drug combination can be suitable. Also any of the aforementioned bioactive agents can be suitable as a second bioactive agent, individually or in combination.

In one example, the bioactive agent can comprise an mRNA vaccine, ODN, or a combination thereof and the second bioactive agent can comprise a chemotherapy drug or drugs.

A second bioactive agent can be any of the aforementioned bioactive agent and can include, for example, an mRNA, a chemotherapy drug, such as one described herein or as known in the art, analgesics/antipyretics (e.g., aspirin, acetaminophen, ibuprofen, naproxen sodium, buprenorphine hydrochloride, propoxyphene hydrochloride, propoxyphene napsylate, meperidine hydrochloride, hydromorphone hydrochloride, morphine sulfate, oxycodone hydrochloride, codeine phosphate, dihydrocodeine bitartrate, pentazocine hydrochloride, hydrocodone bitartrate, levorphanol tartrate, diflunisal, trolamine salicylate, nalbuphine hydrochloride, mefenamic acid, butorphanol tartrate, choline salicylate, butalbital, phenyltoloxamine citrate, diphenhydramine citrate, methotrimeprazine, cinnamedrine hydrochloride, meprobamate and the like); anesthetics (e.g., cyclopropane, enflurane, halothane, isoflurane, methoxyflurane, nitrous oxide, propofol and the like); antiasthmatics (e.g., azelastine, ketotifen, traxanox, amlexanox, cromolyn, ibudilast, montelukast, nedocromil, oxatomide, pranlukast, seratrodast, suplatast tosylate, tiaramide, zafirlukast, zileuton, beclomethasone, budesonide, dexamethasone, flunisolide, triamcinolone acetonide and the like); antibiotics (e.g., neomycin, streptomycin, chloramphenicol, cephalosporin, ampicillin, penicillin, tetracycline and the like); antidepressants (e.g., nefopam, oxypertine, doxepin hydrochloride, amoxapine, trazodone hydrochloride, amitriptyline hydrochloride, maprotiline hydrochloride, phenelzine sulfate, desipramine hydrochloride, nortriptyline hydrochloride, tranylcypromine sulfate, fluoxetine hydrochloride, doxepin hydrochloride, imipramine hydrochloride, imipramine pamoate, nortriptyline, amitriptyline hydrochloride, isocarboxazid, trimipramine maleate, protriptyline hydrochloride and the like); antidiabetics (e.g., biguanides, hormones, sulfonylurea derivatives, and the like); antifungal agents (e.g., griseofulvin, ketoconazole, amphotericin B, nystatin, candicidin and the like); antihypertensive agents (e.g., propanolol, propafenone, oxyprenolol, nifedipine, reserpine, trimethaphan camsylate, phenoxybenzamine hydrochloride, pargyline hydrochloride, deserpidine, diazoxide, guanethidine monosulfate, minoxidil, rescinnamine, sodium nitroprusside, rauwolfia serpentina, alseroxylon, phentolamine mesylate, reserpine and the like); anti-inflammatories (e.g., non-steroidal compounds, such as, indomethacin, naproxen, ibuprofen, ramifenazone, piroxicam and so on, and steroidal compounds, such as, cortisone, dexamethasone, fluazacort, hydrocortisone, prednisolone, prednisone and the like); antineoplastics (e.g., adriamycin, cyclophosphamide, actinomycin, bleomycin, daunorubicin, doxorubicin, epirubicin, mitomycin, methotrexate, fluorouracil, carboplatin, carmustine (BCNU), methyl-CCNU (semustine), cisplatin, etoposide, interferons, camptothecin and derivatives thereof, phenesterine, Taxol and derivatives thereof, taxotere and derivatives thereof, vinblastine, vincristine, tamoxifen, etoposide, piposulfan and the like); antianxiety agents (e.g., lorazepam, buspirone hydrochloride, prazepam, chlordiazepoxide hydrochloride, oxazepam, clorazepate dipotassium, diazepam, hydroxyzine pamoate, hydroxyzine hydrochloride, alprazolam, droperidol, halazepam, chlormezanone, dantrolene and the like); immunosuppressive agents (e.g., cyclosporine, azathioprine, mizoribine, Sirolimus (rapamycin), FK506 (tacrolimus) and the like); antimigraine agents (e.g., ergotamine tartrate, propanolol hydrochloride, isometheptene mucate, dichloralphenazone and the like); sedatives/hypnotics (e.g., barbiturates (e.g., pentobarbital, pentobarbital sodium, secobarbital sodium and the like) or benzodiazapines (e.g., flurazepam hydrochloride, triazolam, tomazeparm, midazolam hydrochloride and the like)); antianginal agents (e.g., R-adrenergic blockers, calcium channel blockers (e.g., nifedipine, diltiazem hydrochloride and the like) and nitrates (e.g., nitroglycerin, isosorbide dinitrate, pentaerythritol tetranitrate, erythrityl tetranitrate and the like)); antipsychotic agents (e.g., haloperidol, loxapine succinate, loxapine hydrochloride, thioridazine, thioridazine hydrochloride, thiothixene, fluphenazine hydrochloride, fluphenazine decanoate, fluphenazine enanthate, trifluoperazine hydrochloride, chlorpromazine hydrochloride, perphenazine, lithium citrate, prochlorperazine and the like); antimanic agents (e.g., lithium carbonate); antiarrhythmics (e.g., bretylium tosylate, esmolol hydrochloride, verapamil hydrochloride, amiodarone, encamide hydrochloride, digoxin, digitoxin, mexiletine hydrochloride, disopyramide phosphate, procainamide hydrochloride, quinidine sulfate, quinidine gluconate, quinidine polygalacturonate, flecamide acetate, tocamide hydrochloride, lidocaine hydrochloride and the like); antiarthritic agents (e.g., phenylbutazone, sulindac, penicillamine, salsalate, piroxicam, azathioprine, indomethacin, meclofenamate sodium, gold sodium thiomalate, ketoprofen, auranofin, aurothioglucose, tolmetin sodium and the like); antigout agents (e.g., colchicine, allopurinol and the like); anticoagulants (e.g., heparin, heparin sodium, warfarin sodium and the like); thrombolytic agents (e.g., urokinase, streptokinase, altoplase and the like); antifibrinolytic agents (e.g., aminocaproic acid); hemorheologic agents (e.g., pentoxifylline); antiplatelet agents (e.g., aspirin, empirin, ascriptin and the like); anticonvulsants (e.g., valproic acid, divalproate sodium, phenyloin, phenyloin sodium, clonazepam, primidone, phenobarbitol, phenobarbitol sodium, carbamazepine, amobarbital sodium, methsuximide, metharbital, mephobarbital, mephenyloin, phensuximide, paramethadione, ethotoin, phenacemide, secobarbitol sodium, clorazepate dipotassium, trimethadione and the like); antiparkinson agents (e.g., ethosuximide and the like); antihistamines/antipruritics (e.g., hydroxyzine hydrochloride, diphenhydramine hydrochloride, chlorpheniramine maleate, brompheniramine maleate, cyproheptadine hydrochloride, terfenadine, clemastine fumarate, triprolidine hydrochloride, carbinoxamine maleate, diphenylpyraline hydrochloride, phenindamine tartrate, azatadine maleate, tripelennamine hydrochloride, dexchlorpheniramine maleate, methdilazine hydrochloride, trimprazine tartrate and the like); agents useful for calcium regulation (e.g., calcitonin, parathyroid hormone and the like); antibacterial agents (e.g., amikacin sulfate, aztreonam, chloramphenicol, chloramphenicol palmitate, chloramphenicol sodium succinate, ciprofloxacin hydrochloride, clindamycin hydrochloride, clindamycin palmitate, clindamycin phosphate, metronidazole, metronidazole hydrochloride, gentamicin sulfate, lincomycin hydrochloride, tobramycin sulfate, vancomycin hydrochloride, polymyxin B sulfate, colistimethate sodium, colistin sulfate and the like); antiviral agents (e.g., interferon γ, zidovudine, amantadine hydrochloride, ribavirin, acyclovir and the like); antimicrobials (e.g., cephalosporins (e.g., cefazolin sodium, cephradine, cefaclor, cephapirin sodium, ceftizoxime sodium, cefoperazone sodium, cefotetan disodium, cefutoxime azotil, cefotaxime sodium, cefadroxil monohydrate, ceftazidime, cephalexin, cephalothin sodium, cephalexin hydrochloride monohydrate, cefamandole nafate, cefoxitin sodium, cefonicid sodium, ceforanide, ceftriaxone sodium, ceftazidime, cefadroxil, cephradine, cefuroxime sodium and the like), penicillins (e.g., ampicillin, amoxicillin, penicillin G benzathine, cyclacillin, ampicillin sodium, penicillin G K, penicillin V K, piperacillin sodium, oxacillin sodium, bacampicillin hydrochloride, cloxacillin sodium, ticarcillin disodium, azlocillin sodium, carbenicillin indanyl sodium, penicillin G procaine, methicillin sodium, nafcillin sodium and the like), erythromycins (e.g., erythromycin ethylsuccinate, erythromycin, erythromycin estolate, erythromycin lactobionate, erythromycin stearate, erythromycin ethylsuccinate and the like), tetracyclines (e.g., tetracycline hydrochloride, doxycycline hyclate, minocycline hydrochloride and the like), and the like); antiinfectives (e.g., GM-CSF); bronchodilators (e.g., sympathomimetics (e.g., epinephrine hydrochloride, metaproterenol sulfate, terbutaline sulfate, isoetharine, isoetharine mesylate, isoetharine hydrochloride, albuterol sulfate, albuterol, bitolterol, mesylate isoproterenol hydrochloride, terbutaline sulfate, epinephrine bitartrate, metaproterenol sulfate, epinephrine, epinephrine bitartrate); anticholinergic agents (e.g., ipratropium bromide); xanthines (e.g., aminophylline, dyphylline, metaproterenol sulfate, aminophylline); mast cell stabilizers (e.g., cromolyn sodium); inhalant corticosteroids (e.g., flunisolide, beclomethasone dipropionate monohydrate and the like), salbutamol, beclomethasone dipropionate (BDP), ipratropium bromide, budesonide, ketotifen, salmeterol, xinafoate, terbutaline sulfate, triamcinolone, theophylline, nedocromil sodium, metaproterenol sulfate, albuterol, flunisolide and the like); hormones (e.g., androgens (e.g., danazol, testosterone cypionate, fluoxymesterone, ethyltostosterone, testosterone enanthate, methyltestosterone, fluoxymesterone, testosterone cypionate and the like); estrogens (e.g., estradiol, estropipate, conjugated estrogens and the like), progestins (e.g., methoxyprogesterone acetate, norethindrone acetate and the like), corticosteroids (e.g., triamcinolone, betamethasone, betamethasone sodium phosphate, dexamethasone, dexamethasone sodium phosphate, dexamethasone acetate, prednisone, methyprednisolone acetate suspension, triamcinolone acetonide, methylprednisolone, prednisolone sodium phosphate methylprednisolone sodium succinate, hydrocortisone sodium succinate, methylprednisolone sodium succinate, triamcinolone hexacatonide, hydrocortisone, hydrocortisone cypionate, prednisolone, fluorocortisone acetate, paramethasone acetate, prednisolone tebulate, prednisolone acetate, prednisolone sodium phosphate, hydrocortisone sodium succinate and the like), thyroid hormones (e.g., levothyroxine sodium); and the like); and the like; hypoglycemic agents (e.g., human insulin, purified beef insulin, purified pork insulin, glyburide, chlorpropamide, glipizide, tolbutamide, tolazamide and the like); hypolipidemic agents (e.g., clofibrate, dextrothyroxine sodium, probucol, lovastatin, niacin and the like); proteins (e.g., DNase, alginase, superoxide dismutase, lipase and the like); nucleic acids (e.g., sense or anti-sense nucleic acids encoding any therapeutically useful protein, including any of the proteins described herein and the like); agents useful for erythropoiesis (e.g., erythropoietin); antiulcer or antireflux agents (e.g., famotidine, cimetidine, ranitidine hydrochloride and the like); antinauseants or antiemetics (e.g., meclizine hydrochloride, nabilone, prochlorperazine, dimenhydrinate, promethazine hydrochloride, thiethylperazine, scopolamine and the like); oil-soluble vitamins (e.g., vitamins A, D, E, K and the like); and as well as other drugs such as mitotane, visadine, halonitrosoureas, anthrocyclines, ellipticine and the like.

Bioactive agents or second bioactive agents can include chemotherapy drugs such as Abemaciclib, Abiraterone Acetate, Abitrexate (Methotrexate), Abraxane (Paclitaxel Albumin-stabilized Nanoparticle Formulation), ABVD, ABVE, ABVE-PC, AC, Acalabrutinib, AC-T, Adcetris (Brentuximab Vedotin), ADE, Ado-Trastuzumab Emtansine, Adriamycin (Doxorubicin Hydrochloride), Afatinib Dimaleate, Afinitor (Everolimus), Akynzeo (Netupitant and Palonosetron Hydrochloride), Aldara (Imiquimod), Aldesleukin, Alecensa (Alectinib), Alectinib, Alemtuzumab, Alimta (Pemetrexed Disodium), Aliqopa (Copanlisib Hydrochloride), Alkeran for Injection (Melphalan Hydrochloride), Alkeran Tablets (Melphalan), Aloxi (Palonosetron Hydrochloride), Alunbrig (Brigatinib), Ambochlorin (Chlorambucil), Amboclorin (Chlorambucil), Amifostine, Aminolevulinic Acid, Anastrozole, Aprepitant, Aredia (Pamidronate Disodium), Arimidex (Anastrozole), Aromasin (Exemestane), Arranon (Nelarabine), Arsenic Trioxide, Arzerra (Ofatumumab), Asparaginase Erwinia chrysanthemi, Atezolizumab, Avastin (Bevacizumab), Avelumab, Axicabtagene Ciloleucel, Axitinib, Azacitidine, Bavencio (Avelumab), BEACOPP, Becenum (Carmustine), Beleodaq (Belinostat), Belinostat, Bendamustine Hydrochloride, BEP, Besponsa (Inotuzumab Ozogamicin), Bevacizumab, Bexarotene, Bexxar (Tositumomab and Iodine I-131 Tositumomab), Bicalutamide, BiCNU (Carmustine), Bleomycin, Blinatumomab, Blincyto (Blinatumomab), Bortezomib, Bosulif (Bosutinib), Bosutinib, Brentuximab Vedotin, Brigatinib, BuMel, Busulfan, Busulfex (Busulfan), Cabazitaxel, Cabometyx (Cabozantinib-S-Malate), Cabozantinib-S-Malate, CAF, Calquence (Acalabrutinib), Campath (Alemtuzumab), Camptosar (Irinotecan Hydrochloride), Capecitabine, CAPOX, Carac (Fluorouracil—Topical), Carboplatin, CARBOPLATIN-TAXOL, Carfilzomib, Carmubris (Carmustine), Carmustine, Carmustine Implant, Casodex (Bicalutamide), CEM, Ceritinib, Cerubidine (Daunorubicin Hydrochloride), Cervarix (Recombinant HPV Bivalent Vaccine), Cetuximab, CEV, Chlorambucil, CHLORAMBUCIL-PREDNISONE, CHOP, Cisplatin, Cladribine, Clafen (Cyclophosphamide), Clofarabine, Clofarex (Clofarabine), Clolar (Clofarabine), CMF, Cobimetinib, Cometriq (Cabozantinib-S-Malate), Copanlisib Hydrochloride, COPDAC, COPP, COPP-ABV, Cosmegen (Dactinomycin), Cotellic (Cobimetinib), Crizotinib, CVP, Cyclophosphamide, Cyfos (Ifosfamide), Cyramza (Ramucirumab), Cytarabine, Cytarabine Liposome, Cytosar-U (Cytarabine), Cytoxan (Cyclophosphamide), Dabrafenib, Dacarbazine, Dacogen (Decitabine), Dactinomycin, Daratumumab, Darzalex (Daratumumab), Dasatinib, Daunorubicin Hydrochloride, Daunorubicin Hydrochloride and Cytarabine Liposome, Decitabine, Defibrotide Sodium, Defitelio (Defibrotide Sodium), Degarelix, Denileukin Diftitox, Denosumab, DepoCyt (Cytarabine Liposome), Dexamethasone, Dexrazoxane Hydrochloride, Dinutuximab, Docetaxel, Doxil (Doxorubicin Hydrochloride Liposome), Doxorubicin Hydrochloride, Doxorubicin Hydrochloride Liposome, Dox-SL (Doxorubicin Hydrochloride Liposome), DTIC-Dome (Dacarbazine), Durvalumab, Efudex (Fluorouracil—Topical), Elitek (Rasburicase), Ellence (Epirubicin Hydrochloride), Elotuzumab, Eloxatin (Oxaliplatin), Eltrombopag Olamine, Emend (Aprepitant), Empliciti (Elotuzumab), Enasidenib Mesylate, Enzalutamide, Epirubicin Hydrochloride, EPOCH, Erbitux (Cetuximab), Eribulin Mesylate, Erivedge (Vismodegib), Erlotinib Hydrochloride, Erwinaze (Asparaginase Erwinia chrysanthemi), Ethyol (Amifostine), Etopophos (Etoposide Phosphate), Etoposide, Etoposide Phosphate, Evacet (Doxorubicin Hydrochloride Liposome), Everolimus, Evista (Raloxifene Hydrochloride), Evomela (Melphalan Hydrochloride), Exemestane, 5-FU (Fluorouracil Injection), 5-FU (Fluorouracil—Topical), Fareston (Toremifene), Farydak (Panobinostat), Faslodex (Fulvestrant), FEC, Femara (Letrozole), Filgrastim, Fludara (Fludarabine Phosphate), Fludarabine Phosphate, Fluoroplex (Fluorouracil—Topical), Fluorouracil Injection, Fluorouracil—Topical, Flutamide, Folex (Methotrexate), Folex PFS (Methotrexate), FOLFIRI, FOLFIRI-BEVACIZUMAB, FOLFIRI-CETUXIMAB, FOLFIRINOX, FOLFOX, Folotyn (Pralatrexate), FU-LV, Fulvestrant, Gardasil (Recombinant HPV Quadrivalent Vaccine), Gardasil 9 (Recombinant HPV Nonavalent Vaccine), Gazyva (Obinutuzumab), Gefitinib, Gemcitabine Hydrochloride, GEMCITABINE-CISPLATIN, GEMCITABINE-OXALIPLATIN, Gemtuzumab Ozogamicin, Gemzar (Gemcitabine Hydrochloride), Gilotrif (Afatinib Dimaleate), Gleevec (Imatinib Mesylate), Gliadel (Carmustine Implant), Gliadel wafer (Carmustine Implant), Glucarpidase, Goserelin Acetate, Halaven (Eribulin Mesylate), Hemangeol (Propranolol Hydrochloride), Herceptin (Trastuzumab), HPV Bivalent Vaccine, Recombinant, HPV Nonavalent Vaccine, Recombinant, HPV Quadrivalent Vaccine, Recombinant, Hycamtin (Topotecan Hydrochloride), Hydrea (Hydroxyurea), Hydroxyurea, Hyper-CVAD, Ibrance (Palbociclib), Ibritumomab Tiuxetan, Ibrutinib, ICE, Iclusig (Ponatinib Hydrochloride), Idamycin (Idarubicin Hydrochloride), Idarubicin Hydrochloride, Idelalisib, Idhifa (Enasidenib Mesylate), Ifex (Ifosfamide), Ifosfamide, Ifosfamidum (Ifosfamide), IL-2 (Aldesleukin), Imatinib Mesylate, Imbruvica (lbrutinib), Imfinzi (Durvalumab), Imiquimod, Imlygic (Talimogene Laherparepvec), Inlyta (Axitinib), Inotuzumab Ozogamicin, Interferon Alfa-2b, Recombinant, Interleukin-2 (Aldesleukin), Intron A (Recombinant Interferon Alfa-2b), Iodine I 131 Tositumomab and Tositumomab, Ipilimumab, Iressa (Gefitinib), Irinotecan Hydrochloride, Irinotecan Hydrochloride Liposome, Istodax (Romidepsin), Ixabepilone, Ixazomib Citrate, Ixempra (Ixabepilone), Jakafi (Ruxolitinib Phosphate), JEB, Jevtana (Cabazitaxel), Kadcyla (Ado-Trastuzumab Emtansine), Keoxifene (Raloxifene Hydrochloride), Kepivance (Palifermin), Keytruda (Pembrolizumab), Kisqali (Ribociclib), Kymriah (Tisagenlecleucel), Kyprolis (Carfilzomib), Lanreotide Acetate, Lapatinib Ditosylate, Lartruvo (Olaratumab), Lenalidomide, Lenvatinib Mesylate, Lenvima (Lenvatinib Mesylate), Letrozole, Leucovorin Calcium, Leukeran (Chlorambucil), Leuprolide Acetate, Leustatin (Cladribine), Levulan (Aminolevulinic Acid), Linfolizin (Chlorambucil), LipoDox (Doxorubicin Hydrochloride Liposome), Lomustine, Lonsurf (Trifluridine and Tipiracil Hydrochloride), Lupron (Leuprolide Acetate), Lupron Depot (Leuprolide Acetate), Lupron Depot-Ped (Leuprolide Acetate), Lynparza (Olaparib), Marqibo (Vincristine Sulfate Liposome), Matulane (Procarbazine Hydrochloride), Mechlorethamine Hydrochloride, Megestrol Acetate, Mekinist (Trametinib), Melphalan, Melphalan Hydrochloride, Mercaptopurine, Mesna, Mesnex (Mesna), Methazolastone (Temozolomide), Methotrexate, Methotrexate LPF (Methotrexate), Methylnaltrexone Bromide, Mexate (Methotrexate), Mexate-AQ (Methotrexate), Midostaurin, Mitomycin C, Mitoxantrone Hydrochloride, Mitozytrex (Mitomycin C), MOPP, Mozobil (Plerixafor), Mustargen (Mechlorethamine Hydrochloride), Mutamycin (Mitomycin C), Myleran (Busulfan), Mylosar (Azacitidine), Mylotarg (Gemtuzumab Ozogamicin), Nanoparticle Paclitaxel (Paclitaxel Albumin-stabilized Nanoparticle Formulation), Navelbine (Vinorelbine Tartrate), Necitumumab, Nelarabine, Neosar (Cyclophosphamide), Neratinib Maleate, Nerlynx (Neratinib Maleate), Netupitant and Palonosetron Hydrochloride, Neulasta (Pegfilgrastim), Neupogen (Filgrastim), Nexavar (Sorafenib Tosylate), Nilandron (Nilutamide), Nilotinib, Nilutamide, Ninlaro (Ixazomib Citrate), Niraparib Tosylate Monohydrate, Nivolumab, Nolvadex (Tamoxifen Citrate), Nplate (Romiplostim), Obinutuzumab, Odomzo (Sonidegib), OEPA, Ofatumumab, OFF, Olaparib, Olaratumab, Omacetaxine Mepesuccinate, Oncaspar (Pegaspargase), Ondansetron Hydrochloride, Onivyde (Irinotecan Hydrochloride Liposome), Ontak (Denileukin Diftitox), Opdivo (Nivolumab), OPPA, Osimertinib, Oxaliplatin, Paclitaxel, Paclitaxel Albumin-stabilized Nanoparticle Formulation, PAD, Palbociclib, Palifermin, Palonosetron Hydrochloride, Palonosetron Hydrochloride and Netupitant, Pamidronate Disodium, Panitumumab, Panobinostat, Paraplat (Carboplatin), Paraplatin (Carboplatin), Pazopanib Hydrochloride, PCV, PEB, Pegaspargase, Pegfilgrastim, Peginterferon Alfa-2b, PEG-Intron (Peginterferon Alfa-2b), Pembrolizumab, Pemetrexed Disodium, Perjeta (Pertuzumab), Pertuzumab, Platinol (Cisplatin), Platinol-AQ (Cisplatin), Plerixafor, Pomalidomide, Pomalyst (Pomalidomide), Ponatinib Hydrochloride, Portrazza (Necitumumab), Pralatrexate, Prednisone, Procarbazine Hydrochloride, Proleukin (Aldesleukin), Prolia (Denosumab), Promacta (Eltrombopag Olamine), Propranolol Hydrochloride, Provenge (Sipuleucel-T), Purinethol (Mercaptopurine), Purixan (Mercaptopurine), Radium 223 Dichloride, Raloxifene Hydrochloride, Ramucirumab, Rasburicase, R-CHOP, R-CVP, Recombinant Human Papillomavirus (HPV) Bivalent Vaccine, Recombinant Human Papillomavirus (HPV) Nonavalent Vaccine, Recombinant Human Papillomavirus (HPV) Quadrivalent Vaccine, Recombinant Interferon Alfa-2b, Regorafenib, Relistor (Methylnaltrexone Bromide), R-EPOCH, Revlimid (Lenalidomide), Rheumatrex (Methotrexate), Ribociclib, R-ICE, Rituxan (Rituximab), Rituxan Hycela (Rituximab and Hyaluronidase Human), Rituximab, Rituximab and Hyaluronidase Human, Rolapitant Hydrochloride, Romidepsin, Romiplostim, Rubidomycin (Daunorubicin Hydrochloride), Rubraca (Rucaparib Camsylate), Rucaparib Camsylate, Ruxolitinib Phosphate, Rydapt (Midostaurin), Sclerosol Intrapleural Aerosol (Talc), Siltuximab, Sipuleucel-T, Somatuline Depot (Lanreotide Acetate), Sonidegib, Sorafenib Tosylate, Sprycel (Dasatinib), STANFORD V, Sterile Talc Powder (Talc), Steritalc (Talc), Stivarga (Regorafenib), Sunitinib Malate, Sutent (Sunitinib Malate), Sylatron (Peginterferon Alfa-2b), Sylvant (Siltuximab), Synribo (Omacetaxine Mepesuccinate), Tabloid (Thioguanine), TAC, Tafinlar (Dabrafenib), Tagrisso (Osimertinib), Talc, Talimogene Laherparepvec, Tamoxifen Citrate, Tarabine PFS (Cytarabine), Tarceva (Erlotinib Hydrochloride), Targretin (Bexarotene), Tasigna (Nilotinib), Taxol (Paclitaxel), Taxotere (Docetaxel), Tecentriq (Atezolizumab), Temodar (Temozolomide), Temozolomide, Temsirolimus, Thalidomide, Thalomid (Thalidomide), Thioguanine, Thiotepa, Tisagenlecleucel, Tolak (Fluorouracil—Topical), Topotecan Hydrochloride, Toremifene, Torisel (Temsirolimus), Tositumomab and Iodine I 131 Tositumomab, Totect (Dexrazoxane Hydrochloride), TPF, Trabectedin, Trametinib, Trastuzumab, Treanda (Bendamustine Hydrochloride), Trifluridine and Tipiracil Hydrochloride, Trisenox (Arsenic Trioxide), Tykerb (Lapatinib Ditosylate), Unituxin (Dinutuximab), Uridine Triacetate, VAC, Valrubicin, Valstar (Valrubicin), Vandetanib, VAMP, Varubi (Rolapitant Hydrochloride), Vectibix (Panitumumab), VeIP, Velban (Vinbiastine Sulfate), Velcade (Bortezomib), Velsar (Vinblastine Sulfate), Vemurafenib, Venclexta (Venetoclax), Venetoclax, Verzenio (Abemaciclib), Viadur (Leuprolide Acetate), Vidaza (Azacitidine), Vinblastine Sulfate, Vincasar PFS (Vincristine Sulfate), Vincristine Sulfate, Vincristine Sulfate Liposome, Vinorelbine Tartrate, VIP, Vismodegib, Vistogard (Uridine Triacetate), Voraxaze (Glucarpidase), Vorinostat, Votrient (Pazopanib Hydrochloride), Vyxeos (Daunorubicin Hydrochloride and Cytarabine Liposome), Wellcovorin (Leucovorin Calcium), Xalkori (Crizotinib), Xeloda (Capecitabine), XELIRI, XELOX, Xgeva (Denosumab), Xofigo (Radium 223 Dichloride), Xtandi (Enzalutamide), Yervoy (Ipilimumab), Yescarta (Axicabtagene Ciloleucel), Yondelis (Trabectedin), Zaltrap (Ziv-Aflibercept), Zarxio (Filgrastim), Zejula (Niraparib Tosylate Monohydrate), Zelboraf (Vemurafenib), Zevalin (Ibritumomab Tiuxetan), Zinecard (Dexrazoxane Hydrochloride), Ziv-Aflibercept, Zofran (Ondansetron Hydrochloride), Zoladex (Goserelin Acetate), Zoledronic Acid, Zolinza (Vorinostat), Zometa (Zoledronic Acid), Zydelig (Idelalisib), Zykadia (Ceritinib), Zytiga (Abiraterone Acetate), or a combination thereof.

In a further example of the method of this invention, a bioactive agent can comprise one or more chemotherapy drugs selected from a taxane, gemcitabine, carboplatin, cisplatin, or a combination thereof, and the second bioactive agent can comprise an ODN, a second chemotherapy drug, one or more anti-PD1 antibodies, anti-PD-L1 antibodies, anti-CTLA-4 antibodies, anti-LAG3 antibodies, anti-TIM-3 antibodies, anti-CD19 antibodies, anti-CD20 antibodies, or a combination thereof.

In one embodiment of the method for treating a disease, a bioactive composition can be introduced to cells in vitro by associating a bioactive composition comprising a biodegradable polymer and a bioactive agent and the cells. In another embodiment, a bioactive composition can be introduced to cells in vivo. A subject can be a mammal or a human. A bioactive composition can be administered with intravenous (IV), intramuscular (IM), subcutaneous (SC) or intradermal (ID) injections, orally, through inhalation, nasally, through an eye, for example, using drops or an ointment, transdermally, for example, using a patch, or a combination thereof. A combination of any aforementioned administering routes can also be suitable.

Any aforementioned bioactive composition or nanoparticles prepared therefrom can be suitable.

As disclosed above and hereunder, a bioactive agent can be any compound with a physiologic or pharmacologic effect, such as, any of the aforementioned RNA, mRNA, RNAi, siRNA, microRNA, oligonucleotide, DNA, oligodeoxynucleotide, protein, peptide, antibody, fragment of antibody, chemical compound, small molecule drug, chemotherapy drug, or a combination thereof.

In the method, a bioactive agent can comprise a vaccine. In one example, a bioactive agent can comprise a DNA or an mRNA vaccine encoding at least one antigen, or a combination of a DNA and an mRNA vaccine thereof. Aforementioned vaccines include a DNA or an mRNA encoding various antigens associated with infections caused by different organisms or pathogens such as viruses, spores, bacteria, fungal, etc. A DNA and/or an mRNA vaccine can also include single or multiple neo-antigens for cancer treatment. In addition, a DNA and/or an mRNA vaccine can also be used for protein regeneration or replacement therapy purposes.

In the method disclosed herein, a bioactive agent can further comprise a second or a subsequent bioactive agent selected from an inhibitor or an activator of a bioprocess. Aforementioned small molecule drugs, antibodies, or a combination thereof, can be suitable.

An instant bioactive composition comprising a bioactive agent can be used in a combination therapy. Different bioactive agents can be attached to one or more biodegradable polymers of interest. Two or more bioactive agents, with each attached to a separate biodegradable polymer of interest, and the two or more bioactive/biodegradable polymer compositions can be mixed or administered simultaneously or reasonable coincidentally. Also, one bioactive agent, such as an ODN, can be associated with a biodegradable polymer and a second bioactive agent that is not associated with a biodegradable polymer of interest. A second bioactive agent can be administered with or without a carrier and administered in any aforementioned mode, such as, oral, parenteral and so on.

This invention is further directed to the use of a biodegradable polymer for delivering an RNA, an mRNA, an RNAi, a siRNA, a microRNA, an oligonucleotide, a DNA, an oligodeoxynucleotide, a protein, a peptide, an antibody, a fragment of an antibody, a chemical compound, a small molecule drug, chemotherapy drug or a combination thereof, into cells or to a subject. A subject can be a human or an animal. Any biodegradable polymers of this invention disclosed herein can be suitable.

This invention is further directed to the use of a bioactive composition for delivering a bioactive agent into cells or a subject. Any bioactive compositions disclosed herein can be suitable.

This invention is further directed to the use of a bioactive composition for vaccinating a subject by administrating a bioactive composition comprising a biodegradable polymer and a bioactive agent into a subject intravenously (IV), intramuscular (IM), subcutaneously (SC), intradermal (ID), orally, through inhalation, nasally, through an eye, transdermally or a combination thereof. Any bioactive compositions disclosed herein can be suitable.

A bioactive composition disclosed herein can comprise a sterile diluent such as water for injection, saline solution, fixed oils, polyethylene glycols, glycerine, glucose, propylene glycol or other synthetic solvents; antibacterial agents such as benzyl alcohol or methyl parabens; antioxidants such as ascorbic acid or sodium bisulfite; chelating agents such as EDTA; buffers such as acetates, citrates or phosphates and agents for the adjustment of tonicity such as sodium chloride or dextrose. pH can be adjusted with acids or bases, such as HCl or NaOH. A bioactive composition of this invention can be enclosed in ampoules, disposable syringes, single dose or multiple dose vials made of glass or plastic.

A bioactive composition disclosed herein can be used as pharmaceutical compositions suitable for injectable use and can include sterile aqueous solutions (where water soluble) or dispersions and sterile powders for extemporaneous preparation of sterile injectable solutions or dispersions. For intravenous administration, suitable carriers include physiological saline, bactoriostatic water, Cremophor EL® (BASF; Parsippany, N.J.) or phosphate-buffered saline (PBS). A bioactive composition must be sterile and should be fluid to the extent that easy syringability exists. A bioactive composition must be stable under conditions of manufacture and storage and must be preserved against contaminating action of microorganisms such as bacteria and fungi. A carrier can be a solvent or dispersion medium containing, for example, water, ethanol, polyol (for example, glycerol, propylene glycol and liquid polyethylene glycol and the like) and suitable mixtures thereof. A proper fluidity can be maintained, for example, by use of a coating such as alecithin, by maintenance of required particle size in the case of a dispersion and by use of surfactants. Prevention of action of microorganisms can be achieved by various antibacterial and antifungal agents, for example, parabens, chlorobutanol, phenol, ascorbic acid and the like. It may be beneficial to include isotonic agents, for example, sugars, polyalcohols such as mannitol, sorbitol or sodium chloride in a composition. Prolonged absorption of injectable compositions can be brought about by including in a composition an agent that delays absorption, for example, a cellulose or a gelatin.

The present invention is further directed to a pharmaceutical composition comprising a biodegradable polymer disclosed herein and at least two bioactive agents, one of which is associated with the biodegradable polymer of interest and the other bioactive agent is not associated with a biodegradable polymer of interest. Hence, for example, a treatment regimen can comprise an ODN associated with a biodegradable polymer and the second bioactive agent can be, for example, an mRNA, or an anti-cancer drug such as aforementioned chemotherapy drug. The two bioactive agents can be administered simultaneously or sequentially.

Any of biodegradable polymers of this invention can be suitable for the pharmaceutical composition.

The at least two bioactive agents can be the same or different. In one further example, one of said two bioactive agents is an ODN attached to a biodegradable polymer and a second bioactive agent is selected form an RNA, an mRNA, an RNAi, a siRNA, an microRNA, an oligonucleotide, a DNA, an oligodeoxynucleotide, a protein, a peptide, an antibody, a fragment of an antibody, a chemical compound, a small molecule drug, or a combination thereof. A second bioactive agent can also be a cancer therapeutic such as a chemotherapy drug.

A pharmaceutical composition disclosed herein can be used for enhancing immune response or for treating cancer in a subject, such as in a human or in an animal.

A bioactive agent can also include diagnostic agents and can include, but not limited to, for example, magnetic resonance imaging contrast agents (e.g., various metal ions, such as, gadolinium based compounds for functional MRI, fluorocarbons, lipid soluble paramagnetic compounds and the like), ultrasound contrast agents, radiocontrast agents, such as, conventional radionuclides, such as, iodine, copper, fluorine, gallium, thallium and the like, which may be complexed with a carrier (e.g., iodo-octanes, halocarbons, renografin and the like), as well as other diagnostic agents which cannot readily be delivered without some physical and/or chemical modification to accommodate the substantially water insoluble nature thereof. Metals and radionuclides can be carried or be bound to a protein, lipid, nucleic acid, chelator, or combinations thereof.

The present invention is further directed to an assay, wherein the assay can comprise a biodegradable polymer composition and a bioactive agent disclosed herein. An assay can be an immunoassay, an enzymatic assay, a nucleic acid based assay, a hybridization assay, or combination thereof. An immunoassay can be a sandwich, competitive, direct, indirect, sequential immunoassay, or a combination thereof. An enzymatic assay can be an enzyme inhibition assay. A nucleic acid assay can be a PCR, gene sequencing, hybridization of nucleic acids assay, hybridization of proteins and nucleic acids, or a combination thereof. A hybridization assay can include hybridization of proteins, hybridization of peptides or hybridization of proteins or peptides with oligos or nucleic acids.

The present invention is further directed to a system for an assay, wherein the system comprises at least one biodegradable polymer of this invention and at least a bioactive agent, and an assay device, wherein the assay is an immunoassay, an enzymatic assay, a nucleic acid based assay, or combination thereof, and wherein the assay is performed on the assay device. In one example, an immunoassay can be a sandwich, competitive, direct, indirect, sequential immunoassay, or a combination thereof and an assay device can comprise an assay strip having an absorption substrate. In another example, an enzymatic assay can be an enzyme inhibition assay. In yet another example, a nucleic acid assay is a PCR, gene sequencing, hybridization of nucleic acids assay, hybridization of proteins and nucleic acids, or a combination thereof, and an assay device can comprise one or more reaction containers. In yet a further example, a hybridization assay can be hybridization of proteins, hybridization of peptides or hybridization of proteins or peptides with oligos or nucleic acids, and wherein an assay device can comprise at least one hybridization container.

This invention is further directed to a controlled release composition comprising one or more biodegradable polymers disclosed herein and at least one bioactive agent, wherein the bioactive agent is encapsulated in the biodegradable polymer.

In examples of controlled release compositions disclosed herein, a bioactive agent can comprise one or more pain relief agents for relieving pain in a patient in need thereof. The one or more pain relief agents can be encapsulated in the biodegradable polymer to form pills, tablet, solutions, injectable solutions, or a combination thereof. The controlled release composition can be administered to the patient intravenously (IV), intramuscular (IM), subcutaneously (SC), intradermal (ID), orally, through inhalation, nasally, through eye, or a combination thereof. Any pain relief agent can be suitable. Water insoluble pain relief agent can particularly suitable. A controlled release composition of this invention can be break done in the patient due to the degradation of the biodegradable polymer resulting in controlled release of the pain relief agent leading to pain relief. An oral pill can be suitable in one example. Biodegradable polymers of this invention that can be degraded by enzymatic process or low pH conditions can be particularly suitable.

In controlled release compositions of this invention, a bioactive agent can comprise one or more pesticides for inhibiting or terminating pest proliferation. Typical pesticides used in agriculture, gardening, home pest control, such as those commercially available and approved by US EPA for commercial or non-commercial use can be suitable. Water insoluble pesticides can be particularly suitable. A controlled release composition of this invention can be break done in environment, such as in soil, due to the degradation of the biodegradable polymer resulting in controlled release of pesticides.

In controlled release compositions of this invention, a bioactive agent can comprise one or more herbicides for inhibiting or terminating vegetation growth. Typical herbicides used in agriculture, gardening, such as those commercially available and approved by US EPA for commercial or non-commercial use can be suitable. Water insoluble herbicides can be particularly suitable. A controlled release composition of this invention can be break done in environment, such as in soil, due to the degradation of the biodegradable polymer resulting in controlled release of herbicides.

In controlled release compositions of this invention, a bioactive agent can comprise one or more fertilizers for promoting growth of vegetation. Typical fertilizer used in agriculture, gardening, such as those commercially available can be suitable. Water insoluble fertilizer can be particularly suitable. A controlled release composition of this invention can be break done in environment, such as in soil, due to the degradation of the biodegradable polymer resulting in controlled release of fertilizer.

One advantage of this invention is that the biodegradable polymer can have higher molecular weight and high cationic charge sufficient for enhancing the delivering a bioactive agent into cells or other biosystems, and at the same time, the polymer can be readily degraded in biosystems to reduce cytotoxicity. The cationic components can be released from the polymer once the biodegradable bonds are cleaved in a biosystem resulting in reduced toxicity. In addition, by using different amine monomers, polymer cores, layers of amine functional groups, amounts of amine groups on the polymer surface and different polymerization procedures as disclosed herein, a biodegradable polymer of this invention can have flexible and diversifying cationic components that can provide easy adjustment of desired properties, such as, but not limited to, levels of positive charge, desired nanoparticle size, functional groups for attaching the bioactive agent, and so on. In general, larger molecular weight of a biodegradable polymer can help to enhance efficiency of delivery of bioactive agent into biosystems, while smaller cationic component can help to reduce cytotoxicity. The biodegradable polymer of this invention can be easily adjusted to have appropriate overall polymer size and the sizes of individual cationic components as suitable for particular uses.

Another advantage of the invention is that biodegradable polymers, the bioactive compositions and nanoparticles of the invention are soluble or dispersible in aqueous solution and can be easily mixed with other ingredients to produce pharmaceutical compositions for treating diseases, such as cancers, infectious diseases, or provide vaccination.

Yet another advantage is that a biodegradable polymer can be modified to be attached or conjugated to other bioactive agents, or multiple different bioactive agents, such as multiple drugs, ligands and antibodies that can make targeted drug delivery possible.

A further advantage of this invention is that a biodegradable polymer can be constructed of biologic molecules, such as, amino acids, which can be degraded into the component amino acids or small peptides that can be utilized by human or animal as nutrition or as building blocks for biosynthesis.

### EXAMPLES

The present invention is further defined in the following Examples. It should be understood that the Examples, while indicating embodiments of the invention, are given by way of illustration only. From the above discussion and the Examples, one skilled in the art can ascertain essential characteristics of this invention, and without departing from the spirit and scope thereof, can make various changes and modifications of the invention to adapt to various uses and conditions.

### Materials

Materials were purchased from Dendritech, Sigma-Aldrich, Quanta BioDesign, Bio-Synthesis Inc., Integrated DNA Technologies Inc., PolySciences Inc., ThermoFisher, Promega, InvivoGen USA, Alamanda Polymers, Hyclone (GE Healthcare Life Sciences), or other appropriate commercial sources.

### Synthesis of Polymers

1. General Procedures for Growth of PEI from Polylysine

A poly-L-lysine can be reacted with ethyleneimine bromide to attach PEI onto the poly-L-lysine via polycondensation growth in the presence of a base, such as diisopropylethylamine. Polymer properties, such as molecular weight, size of polymer chain, degree of branching, or a combination thereof, can be controlled as a design choice, for example, by altering reagents and or reaction conditions. The reaction is schematically shown below:
Poly-L-Lysine (PLK)+BrCH₂CH₂NH₂—HBr+Base→PLK-PEI
   2. General Procedures for Attachment of Preformed PEI or EI onto Poly-L-Lysine

A poly-L-lysine can be reacted with SMCC to produce a modified poly-L-lysine (PLK-MAL). A preformed PEI can be reacted with Traut's agent to generate thio-modified PEI, PEI-SH. Then the PLK-MAL and PEI-SH are reacted to form a biodegradable polymer. The reaction is schematically shown below:
Poly-L-Lysine+SMCC→PLK-MAL
PEI+Traut's reagent→PEI-SH+PLK-MAL-→PLK-PEI.

The PLK-MAL can also react with cysteamine to produce amine modified polylysine PLK-EI as shown below:
PLK-MAL+Cysteamine→PLK-EI.

### Example 1. Polyethyleneimine (PEI) Modified with Poly-L-Lysine (PEI-PLK)

Cationic biodegradable polymer polyethyleneimine-polylysine (PEI-PLK) was prepared by reacting 145 mg of branched polyethyleneimine (MW 1800 Dalton) in 4.03 g of DMSO with 1.304 g of Boc-Lys(Boc)-OSu in 4.32 g of DMSO at room temperature for 4 hours. The mixture was then precipitated, deprotected and then dissolved in DMSO to produce a lysine modified PEI (PEI18-Lysine) that comprises multiple lysine residues on the polymer. One half of the PEI-lysine from the step above was mixed with 2.05 g of Boc-Lys(Boc)-OSu and diisopropylethylamine and reacted for 4 hours. The reaction mixture was precipitated and deprotected by reacting with methylenechloride and trifluoracetic acid at room temperature for 1 hour. The reaction mixture above was air dried with blowing nitrogen at 50° C. to produce the cationic biodegradable polymer having polyethyleneimine (1800 Da) modified with two layers of lysine (PLK2), herein referred to as Den(PEI18-PLK2).

### Example 2. Den(PEI-PLK) Modified with Amine End Group

One half of the Den(PEI18-PLK2) prepared above was mixed with 1.79 g of bromoethylamine in methanol and 1.65 g of diisopropylethylamine, reacted at room temperature for 2 hours. The reaction mixture was precipitated with diethylether. The supernatant was removed. The precipitated contents were dialyzed against water. The resulted polymer water solution was rotary evaporated to dryness to produce a dendrimer PEI-PLK-EI/PEI polymer that has a branched PEI core (1800 Da) modified with two layers of lysine (PLK2) and at least one lysine amine end group modified with one or more ethylamine (EA), herein referred to as Den(PEI18-PLK2-EI/PEI).

### Example 3. Preparation of Den(PEI12-PLK) with 2-3 Layers of Lysine

A branched PEI (bPEI) core of 1200 Da (PEI12), 2.58 g in 10 ml of DMSO was reacted with 19.99 g of Boc-Lys(Boc)-OSu in 40 ml of DMSO at room temperature for 22 hours. The mixture was precipitated, deprotected and then dissolved in DMSO to produce a lysine modified PEI (PEI12-Lysine) that comprises multiple lysine residues on the polymer.

The PEI12-lysine (1.98 g) from the step above was mixed with 9.51 g of Boc-Lys(Boc)-OSu and diisopropylethylamine and reacted for 14 hours. The reaction mixture was precipitated and deprotected by reacting with methylene chloride and trifluoracetic acid at room temperature for 2.5 hours. Solvent was removed in vacuum to produce the cationic biodegradable polymer polyethyleneimine modified with two layers of lysine (PLK2), herein referred to as Den(PEI12-PLK2).

Then 1.20 g of the PEI12-PLK2 from the step above was mixed with 5.33 g of Boc-Lys(Boc)-OSu and diisopropylethylamine in DMSO and reacted for 22 hours. The reaction mixture was precipitated, deprotected dried to produce a dendritic cationic biodegradable polymer polyethyleneimine modified with three layers of lysine (PLK3) (PEI12-PLK3), herein referred to as Den(PEI12-PLK3). The cationic biodegradable polymer polyethyleneimine modified with different layers of lysine were optionally dialyzed against membrane prior to subsequent modifications. The dendrimer Den(PEI12-PLK3) has a calculated PEI core of about 1200 Da, a first layer of about 21 lysine residues, a second layer of about 42 lysine residues and a third layer of about 84 lysine residues schematically shown as bPEI12-Lys21-Lys42-Lys84.

Polymers having different bPEI cores, such as PEI16 (MW 600 Da), PEI25 (MW 25 KDa) or others were produced using the same procedure described above starting from a respective bPEI core and different number of layers of lysine residues, herein referred to as Den(PEI6-PLK2), Den(PEI6-PLK3), Den(PEI25-PLK2), Den(PEI25-PLK3), respectively.

### Example 4. Preparation of Den(PEI12-PLK-EI) with One or More EI

The PEI12-PLK2 (127 mg) prepared above was then reacted with 311 mg of 2-(boc-amino)ethyl bromide in methanol and 0.4 mL of diisopropylethylamine, reacted at room temperature for 69 hours. The reaction mixture was precipitated with diethyl ether. The precipitated contents were deprotected, dried and dialyzed against water. After dialysis, retained polymer in water solution was rotary evaporated to dryness to produce a dendritic polymer Den(PEI-PLK-EI) that has a branched PEI core modified with two layers of lysine (PLK2) and at least one lysine amine end group modified with one ethylamine (EA), herein referred to as Den(PEI12-PLK2-EI).

The PEI-PLK modified with three layers of lysine, Den(PEI12-PLK3) (106 mg) prepared above, was mixed with 241 mg of 2-(boc-amino)ethyl bromide in methanol and 0.32 mL of diisopropylethylamine, reacted at room temperature for 86 hours. The reaction mixture was precipitated. The precipitated contents were deprotected, dried and dialyzed against water. After dialysis, the retained polymer in water solution was rotary evaporated to dryness to produce a dendritic PEI-PLK-EI polymer that has a branched PEI core modified with three layers of lysine (PLK3) and at least one lysine amine end group modified with one ethylamine (EA), herein referred to as Den(PEI12-PLK3-EI).

The PEI-PLK modified with two layers of lysine, PEI12-PLK2 (133 mg) prepared above was mixed with 2.55 g of bromoethylamine in methanol and 5 mL of diisopropylethylamine, reacted at room temperature for 86 hours. The reaction mixture was precipitated, dried and dialyzed against water as described above. After dialysis, the retained polymer in water solution was rotary evaporated to dryness to produce a dendritic PEI-PLK-EI/PEI polymer that has a branched PEI core modified with two layers of lysine (PLK2) and at least one lysine amine end group modified with one or more ethylamine (EA), herein referred to as Den(PEI12-PLK2-EI/PEI), with calculated molecular weight of 16 KDa.

### Example 5. Preparation of Den(PEI-PLK-C18)

Stearic acid (0.479 g) and N-hydroxysuccinimide (NHS, 0.339 g) were combined in 10 mL of DMF (dimethylformamide), followed by the addition of 1-ethyl-3-(3-dimethylaminopropyl)carbodiimide (1.16 g) and diisopropylethylamine (0.26 mL). The solution mixture was stirred at room temperature for 18 hours. The reaction mixture was precipitated in water and the solids were filtered and dried under vacuum to provide NHS activated ester of stearic acid.

The PEI-PLK modified with two layers of lysine, Den(PEI12-PLK2) (60 mg) prepared above was mixed with 5.8 mg of NHS activated ester of stearic acid in 2 mL of DMF, reacted at room temperature for 16 hours. The reaction mixture was precipitated with diethyl ether. The supernatant was removed and the precipitated contents were dissolved in water, dialyzed and dried to produce a PEI-PLK-C18 polymer that has a branched PEI core modified with two layers of lysine (PLK2) and at least one lysine amine end group modified with at least one stearic acid, herein referred to as Den(PEI12-PLK2-C18).

### Example 6. Preparation of Den(PEI-PLK-EI-C18)

The Den(PEI12-PLK2-EI) prepared above (36.5 mg) was mixed with 2.6 mg of NHS activated ester of stearic acid in 2 mL of DMF, reacted at room temperature for 16 hours. The reaction mixture was precipitated with diethyl ether. The supernatant was removed and the precipitated contents were dissolved in water, dialyzed and dried to produce a dendritic PEI-PLK-EI-C18 polymer that has a branched PEI core modified with two layers of lysine (PLK2) and at least one lysine amine end group modified with one ethylamine (EA), with at least one ethylamine further modified with at least one stearic acid, herein referred to as Den(PEI12-PLK2-EI-C18).

### Example 7. Preparation of Den(PLK-PLK-PEI)

Linear polylysine with a molecular weight of 13.5 KDa (66.9 mg) and Boc-Lys(Boc)-OSu (192 mg) were dissolved in DMSO (3 mL) followed by the addition of diisopropylethylamine (0.143 mL). The reaction mixture was stirred at 40° C. for 24 hours and was stopped by adding diethyl ether (30 mL). The bottom layer was washed with additional diethyl ether. The crude product was dissolved in 6 mL of DCM/TFA (1:1, v/v) and stirred under nitrogen for 2 hours.

Solvents were then removed to produce a polymer product having a linear polylysine backbone with additional lysine residues attached thereon, herein referred to as PLK-Lys. One half of the product was dissolved in methanol (3 mL), followed by the addition of bromoethylamine (0.777 g) and diisopropylethylamine (1.32 mL). The reaction mixture was stirred at room temperature for 64 hours. The reaction mixture was then precipitated, dried and dialyzed against water as described above. After dialysis, the retained polymer in water solution was rotary evaporated to dryness to produce a branched polymer that has a linear polylysine backbone with additional lysine residues attached thereon and at least one of lysine amine end groups further modified with one or more ethylamine (EA), herein referred to as PLK-Lys-PEI, with calculated molecular weight of over 36 KDa.

### Example 8. Degradation of the Polymer

Polymers prepared above were treated with trypsin or protease (0.25 mg/mL-25 mg/mL) for 2-72 hours in tris-buffer (pH 8.0) or in 1×PBS buffer (pH 7.5-8.0) at 37° C. temperature. Degradation of polymers were detected by changes of polymer sizes, charge or a combination thereof, as-evidenced by shifts of Size Exclusion Chromatography (SEC) traces towards longer retention time, broadening of peak width, reduction in peak height, and emerging of lower molecular weight peaks.

### Example 9. Polymer Encapsulated Oligodeoxynucleotides (ODN) (Non-Covalent)

Oligodeoxynucleotides (ODN) were purchased from commercial sources. Biodegradable polymers produced above were mixed with an ODN in an aqueous solution to form a desired encapsulate at various polymer to ODN ratios (nitrogen to phosphor ratio) in a range of from 1:10 to 10:1. Some encapsulates were used in assays described below. For cell culture assays, polymer and ODN were prepared in culture media suitable for the cells.

### Example 10. Polymer Toxicity

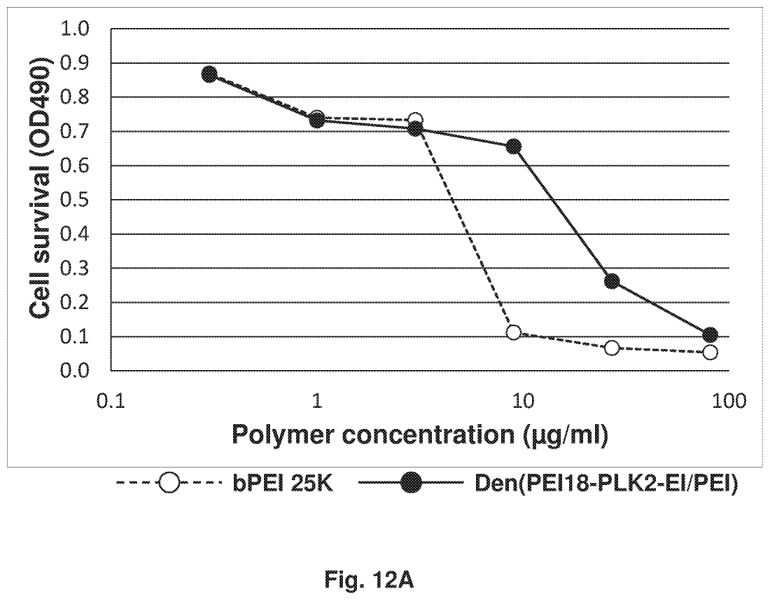

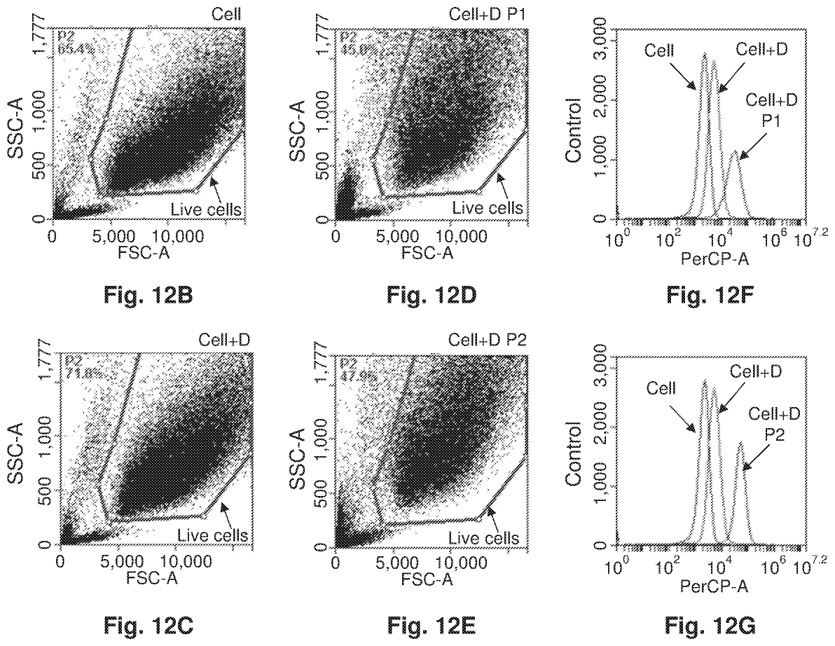

About 2×10⁴ H460 cells were plated in 96 well, cultured in regular media (RPMI) supplemented with a control polymer dendritic branched polyethyleneimine (bPEI 25K) or a biodegradable dendrimer Den(PEI18-PLK2-EI/PEI) prepared in Example 2 above for 48 hours. Cell survival was performed using Promega's CellTiter 96® AQueous One Solution Cell Proliferation Assay (Available from Promega Corporation, Madison, Wis., USA, under respective registered trademark). Representative results are shown in FIG. 12A. The biodegradable Den(PEI18-PLK2-EI/PEI) dendrimer exhibited lower cytotoxicity compared to the control polymer.

### Example 11. Cellular Delivery of mRNA

About 2×10⁴ H460 cells were plated in 96 well, cultured in regular media (RPMI) supplemented with 100 ng of Luciferase mRNA and a polymer from Example 4 Den(PEI12-PLK2-EI/PEI) above as specified in Table 1 at Nitrogen:Phosphate charge ratio of 12:1 for 48 hours. Luciferase activities were measured using a commercially luciferase assay. Control was done with 100 ng of Luciferase mRNA only without polymer. Representative results are shown in Table 1.

TABLE 1
Polymer mediated mRNA delivery into cells.

| | Luciferase |
|---|---|
| Polymer for mRNA delivery | activity |
| Den(PEI-PLK-EI/PEI) | 2023 |
| Control | 106 |

### Example 12. Neo-Antigen and mRNA Vaccine

Neo-antigens representing MHC Class II were produced according to published references (Kreiter, et al., Nature, Vol. 520, 692, 2015, doi:10.1038/nature14426) by construction of plasmids having desired coding regions, a 5′-untranslated region (5′-UTR) and one or more 3′-untranslated regions (3′-UTR). The mRNA was obtained from in vitro transcription using standard commercially available kits. The transcribed mRNA encoding the MHC Class II neo-antigens can be mixed with a biodegradable polymer from Example 2 or 3 in solution to form nanoparticles. The nanoparticles can be used to transfect cells as described above or directly for injection into animals. The nanoparticles can also be lyophilized to form a dry powder for enhanced storage stability. Lyophilized nanoparticles can be reconstituted in saline for injection into animals. Nanoparticles can also be mixed with additional materials, such as an adjuvant, before injection.

All references cited herein are herein incorporated by reference in its entirety.

### Example 13. Cellular Delivery of DNA

About 2×10⁴ H460 cells were plated in 96 well, cultured in regular media (RPMI) supplemented with 100 ng of Luciferase expression plasmid DNA and a polymer from Example 4 Den(PEI12-PLK2-EI/PEI) above as specified in Table 2 at Nitrogen:Phosphate charge ratio of 12:1 for 48 hours. Luciferase activities were measured using a commercially luciferase assay. Control was done with 100 ng of Luciferase DNA only without polymer. Representative results are shown in Table 2.

TABLE 2
Polymer mediated DNA delivery into cells.

| | Luciferase |
|---|---|
| Polymer for DNA delivery | activity |
| Den(PEI18-PLK2-EI/PEI) | 519 |
| Control | 59 |

### Example 14. Cellular Delivery of Oligonucleotides

Lung cancer H460 cell line was cultured in RPMI media supplemented with 10% FBS (Fetal Bovine Serum) and penicillin/streptomycin. About 2×10⁵ cells were suspended in 150 μl of media for each assay. Two nmol of a 20-mer oligo-nucleotide covalently linked with a Cy3 dye were mixed with one of the biodegradable polymers prepared above with at a selected nitrogen (N) to phosphor (P) charge ratio in 20 μl of opti-mem for 10 minutes. Then the oligo-polymer mixture was added into the cell suspension. Each of the cell mixtures was incubated at room temperature for 1.5 hours. Then cells were washed once with 1 ml of fresh culture media and resuspended in 400 μl of opti-mem for FACS (fluorescence activated cell sorting) analysis. About 50,000 cells were acquired and the fluorescence intensity of FL-3 channel were plotted (FIG. 12B-FIG. 12E). Live cells were gated as indicated. Florescence intensities of the gated live cells are shown in FIG. 12F and FIG. 12G, respectively. Percentages of live cells in samples: 65% for cells alone (FIG. 12B), 71% for cells treated with oligo-dye (FIG. 12C), about 45% to 47% for cells treated with oligo-dye and polymer (FIG. 12D and FIG. 12E).

As shown in FIG. 12B, Control FACS profile of H460 cells only (Cell); FIG. 12C, Control FACS profile of cells treated with the 20-mer oligo linked to Cy3 dye (Cell+D); FIG. 12D, cells treated with a 20-mer oligo linked Cy3 dye and a dendrimer from Examples 3, Den(PEI12-PLK3-EI168) (Cell+D P1) at a N:P ratio of 1:1; FIG. 12E, cells treated with a 20-mer oligo linked Cy3 dye and a dendrimer from Examples 6, Den(PEI12-PLK2-EI-C18) (Cell+D P2) at a N:P ratio of 0.2:1; FIG. 12F, florescence intensity profiles of live cells (Cell, Cell+D and Cell+D P1); and FIG. 12G, florescence intensity profiles of live cells (Cell, Cell+D and Cell+D P2).

1.-26. (canceled)

27. A bioactive composition comprising a biodegradable polymer and at least one bioactive agent,
wherein said biodegradable polymer comprises two or more cationic components and at least one biodegradable bond formed by biomolecules, wherein said two or more cationic components are separated by at least one of said at least one biodegradable bond, wherein said biodegradable polymer comprises at least one side chain; and wherein said two or more cationic components are attached to said biomolecules covalently,
at least one of said two or more cationic components comprises at least one end amine group selected from the group consisting of —N(CH₂)NH₂, —N(CH₂)₂NH₂, —N(CH₂)₃NH₂, —N(CH₂)N⁺H₃, —N(CH₂)₂N⁺H₃,
—N(CH₂)₃N⁺H₃ and a combination thereof,
said at least one biodegradable bond is in the backbone of said biodegradable polymer, in at least one side chain of said at least one side chain of said biodegradable polymer, or in both said backbone and at least one side chain of said biodegradable polymer,
said at least one biodegradable bond comprises a peptide bond, an ester bond, a reducible disulfide bond or a combination thereof, and
said biomolecules comprise an amino acid, a lysine (Lys), a modified Lys, a glutamic acid (Glu), a modified Glu, an aspartic acid (Asp), a modified Asp, an arginine (Arg), a modified Arg, a linear polyLys, a branched polyLys, a linear polyGlu, a branched polyGlu, a linear polyArg, a branched polyArg or a combination thereof.

28. (canceled)

29. The bioactive composition of claim 27, wherein said at least one bioactive agent comprises an RNA, an mRNA, an RNAi, an siRNA, a microRNA, an oligonucleotide, a DNA, an oligodeoxynucleotide, a protein, a peptide, an antibody, a fragment of an antibody, a chemical compound, a chemotherapy drug, a small molecule drug, or a combination thereof.

30. The bioactive composition of claim 27, wherein said at least one bioactive agent comprises an antigen.

31. The bioactive composition of claim 27, wherein the said at least one bioactive agent comprises a DNA or an mRNA encoding at least one antigen, or a combination of the DNA and the mRNA encoding at least one antigen.

32. The bioactive composition of claim 27, wherein said at least one bioactive agent comprises a first oligodeoxynucleotide (ODN) attached to said biodegradable polymer through a non-covalent linkage.

33. The bioactive composition of claim 27, wherein said at least one bioactive agent further comprises an inhibitor or an activator of a bioprocess.

34. The bioactive composition of claim 27, wherein said biodegradable polymer and said at least one bioactive agent form nanoparticles in a range of from 1 nm to 1000 nm and said nanoparticles are soluble or dispersible in an aqueous solution.

35. The bioactive composition of claim 27 further comprising a polymer selected from the group consisting of a linear polymer, a branched polymer, a block copolymer, a graft copolymer, a dendrimer and a combination thereof, and wherein said biodegradable polymer and said polymer are the same or are different.

36. The bioactive composition of claim 35 further comprising a second oligodeoxynucleotide (ODN) attached to said biodegradable polymer through a covalent linkage, attached to said polymer through a covalent linkage, or attached to both through covalent linkages.

37. The bioactive composition of claim 35, wherein said polymer comprises a cationic polymer.

38. (canceled)

39. The bioactive composition of claim 27, wherein said bioactive composition comprises at least two bioactive agents, wherein a first of said at least two bioactive agents comprises a first RNA, mRNA, RNAi, siRNA, microRNA, oligonucleotide, DNA, oligodeoxynucleotide, chemical compound, chemotherapy drug, small molecule drug, a protein, a peptide, an antibody, a fragment of an antibody or a combination thereof; and a second of said at least two bioactive agents comprises a second RNA, mRNA, RNAi, siRNA, microRNA, oligonucleotide, DNA, oligodeoxynucleotide, chemical compound, chemotherapy drug, small molecule drug, protein, peptide, antibody, a fragment of an antibody or a combination thereof.

40. (canceled)

41. The bioactive composition of claim 27 further comprising a carrier, and said bioactive composition comprising a carrier comprises a pharmaceutical composition for treating a disease of a subject in need thereof.

42. (canceled)

43. The bioactive composition of claim 27 further comprising a targeting agent for targeting said bioactive composition to at least one target, wherein said targeting agent comprises a physical targeting agent, a chemical targeting agent, a biological target agent or a combination thereof.

44. (canceled)

45. The bioactive composition of claim 43, wherein said at least one target comprises a biosystem selected from the group consisting of cells in vitro, cells in vivo, nuclei of cells, cytoplasm of cells, extracellular matrix, tissues, body fluid of a subject, blood of the subject, an organ of the subject, a tumor of the subject, one or more cells selected from the group consisting of T cells, B cells, NK (natural killer) cells, cancer cells, tumor cells, antigen presenting cells (APC), dendritic cells (DC), neutrophils, macrophages, lymphocytes, monocytes and a combination thereof and a combination thereof.

46.-48. (canceled)

49. A method for treating a disease of a subject in need thereof, said method comprising the step of:
introducing the bioactive composition of claim 27 to the subject, wherein said bioactive composition is introduced to the subject intravenously (IV), intramuscularly (IM), subcutaneously (SC), intradermally (ID), orally, through inhalation, nasally, ocularly or a combination thereof.

50. (canceled)

51. The method of claim 49 further comprising the step of:
subsequently, introducing a second bioactive agent to the subject after the bioactive composition is introduced.

52. The method of claim 51, wherein said second bioactive agent comprises a second RNA, a second mRNA, a second RNAi, a second siRNA, a second microRNA, a second oligonucleotide, a second DNA, a second oligodeoxynucleotide, a second protein, a second peptide, a second antibody, a second fragment of an antibody, a second chemical compound, a second small molecule drug, or a combination thereof, and wherein said bioactive agent and said second bioactive agent are the same or different.

53. The method of claim 51, wherein said bioactive agent comprises an oligodeoxynucleotide (ODN) and said second bioactive agent comprises said RNA, mRNA, RNAi, siRNA, microRNA, oligonucleotide, DNA, oligodeoxynucleotide, protein, peptide, antibody, fragment of the antibody, chemical compound, small molecule drug, or a combination thereof.

54. The method of claim 51, wherein said bioactive agent comprises one or more chemotherapy drugs selected from the group consisting of a taxane, gemcitabine, carboplatin, cisplatin and a combination thereof, and said second bioactive agent comprises an oligodeoxynucleotide (ODN), a second chemotherapy drug, anti-PD1 antibodies, anti-PD-L1 antibodies, anti-CTLA-4 antibodies, anti-LAG3 antibodies, anti-TIM-3 antibodies, anti-CD19 antibodies, anti-CD20 antibodies, or a combination thereof.

55. The method of claim 54, wherein said bioactive composition or said second bioactive agent each is independently introduced to the subject intravenously (IV), intramuscularly (IM), subcutaneously (SC), intradermally (ID), orally, through inhalation, nasally, ocularly, or a combination thereof.

56-64. (canceled)

65. The bioactive composition of claim 27, wherein said biomolecules consist of lysine or polylysine.

66. The bioactive composition of claim 27, wherein said biodegradable polymer comprises a molecular weight of from 1,000 Da to about 1,500,000 Da and wherein each of said two or more cationic components has a molecular weight (MW) of from about 40 Da to about 5,000 Da.

67. The bioactive composition of claim 27, wherein each of said two or more cationic components independently comprises a linear polymer, a branched polymer, a hyperbranched polymer, a graft polymer, a block polymer, a dendrimer, or a combination thereof.

68. The bioactive composition of claim 27, wherein at least one of said two or more cationic components comprises lysine, polylysine, alkyleneimine, polymerized alkyleneimine, ethyleneimine, polymerized ethyleneimine (PEI), propyleneimine, polymerized propyleneimine (PPI), polymerized amidoamine (PAMAM), tris(2-aminoethyl)amine (TREN), polymerized tris(2-aminoethyl)amine, polyalkylamine, polyallylamine or a combination thereof.

69. The bioactive composition of claim 27, wherein said biodegradable polymer comprises one or more polymer segments, each having a formula: 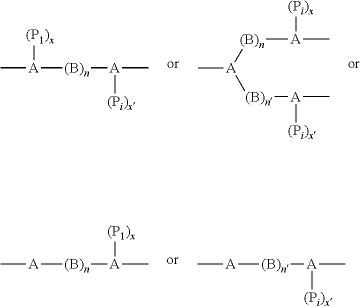
or a combination thereof, wherein,
n and n′ each is an integer ≥0;
x and x′ each is an integer ≥1;
A is a biomolecule;
B is selected from the group consisting of A, a linear polymer component comprising A, a branched polymer component comprising A, a dendrimer component comprising A and a combination thereof,
wherein each of said one or more polymer segments comprises at least one of said at least one biodegradable bond; and
P comprises a cationic component, P₁, P₂, P₃ through Pᵢ comprise said two or more cationic components, and wherein said two or more P₁ through Pᵢ are the same or are different.

70. The bioactive composition of claim 27, wherein said biodegradable polymer further comprises a polymer core comprising 2 or more branching reactive sites, and two or more polymer segments each of which is attached to said polymer core at one of said two or more branching reactive sites, and wherein said polymer core comprises a linear polymer, a branched polymer, a dendrimer, or a combination thereof, wherein said polymer core has a molecular weight of from 40 Da to 5,000 Da.

71. The bioactive composition of claim 70, wherein said polymer core comprises lysine, polylysine, polyaspartic acid, polyglutamic acid, polymerized alkyleneimine, alkyldiamine, ethylenediamine, polymerized ethyleneimine (PEI), propyleneimine, propylenediamine, polymerized propyleneimine (PPI), polymerized amidoamine (PAMAM), tris(2-aminoethyl)amine (TREN), polymerized tris(2-aminoethyl)amine, polyalkylamine, polyallylamine, polyol or a combination thereof.

72. The bioactive composition of claim 70, wherein said biodegradable polymer comprises a formula: 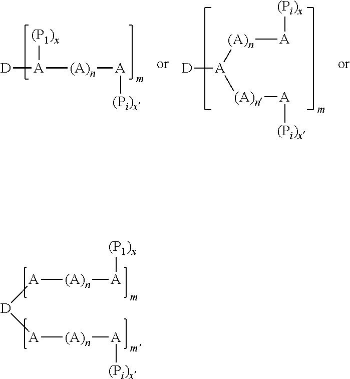
or a combination thereof, wherein,
n and n′ each is an integer ≥0;
x and x′ each is an integer ≥1;
A is a biomolecule;
P comprises a cationic component, P₁, P₂, P₃ through Pᵢ comprise said two or more cationic components, and wherein said two or more P₁ through Pᵢ are the same or are different;
D is said polymer core; and
m and m′ each is an integer ≥0 and m+m′≥2.

73. The bioactive composition of claim 27, wherein said biodegradable polymer further comprises at least one hydrocarbon chain of from 3 to 30 carbon atoms (C₃-C₃₀), wherein said at least one hydrocarbon chain is covalently attached to said biodegradable polymer, said at least one hydrocarbon chain is attached to one of said biomolecules, one of said two or more cationic components, or a combination thereof, and said at least one hydrocarbon chain is a reacted unsaturated fatty acid, a saturated fatty acid, an epoxide derivative of said unsaturated fatty acid, an epoxide derivative of said saturated fatty acid or a combination thereof.

74. The bioactive composition of claim 73, wherein said at least one hydrocarbon chain comprises a reacted propionic acid, butyric acid, valeric acid, caproic acid, enanthic acid, caprylic acid, pelargonic acid, capric acid, undecylic acid, lauric acid, tridecylic acid, myristic acid, pentadecylic acid, palmitic acid, margaric acid, stearic acid, nonadecylic acid, arachidic acid, heneicosylic acid, behenic acid, tricosylic acid, lignoceric acid, pentacosylic acid, cerotic acid, heptacosylic acid, montanic acid, nonacosylic acid, melissic acid, henatriacontylic acid, lacceroic acid, psyllic acid, geddic acid, ceroplastic acid, hexatriacontylic acid, heptatriacontanoic acid, octatriacontanoic acid, α-linolenic acid, stearidonic acid, eicosapentaenoic acid, docosahexaenoic acid, linoleic acid, linolelaidic acid, γ-linolenic acid, dihomo-γ-linolenic acid, arachidonic acid, docosatetraenoic acid, palmitoleic acid, vaccenic acid, paullinic acid, oleic acid, elaidic acid, gondoic acid, erucic acid, nervonic acid, mead acid, an isomer derivative thereof, an epoxide derivative thereof or a combination thereof.

75. The bioactive composition of claim 74, wherein said biomolecules consist of lysine or polylysine and said two or more cationic components comprise ethyleneimine, polymerized ethyleneimine (PEI), propyleneimine, polymerized propyleneimine (PPI), polymerized amidoamine (PAMAM) or a combination thereof.
